(19) United States
(12) Patent Application Publication
(10) Pub. No.: US 2026/0250287 A1
(43) Pub. Date: Aug. 27, 2026

(54) CXCR7 RECEPTOR MODULATOR AND USE THEREOF

(71) Applicant: ILEADBMS CO., LTD., Hwaseong-Si (KR)

(72) Inventors: Yoon-Suk LEE, Hwaseong-si (KR); Yunhee KIM, Yongin-si (KR); Eunhee CHAE, Incheon (KR)

(21) Appl. No.: 19/642,165

(22) Filed: Apr. 8, 2026

Related U.S. Application Data
(63) Continuation-in-part of application No. PCT/KR2024/018655, filed on Nov. 22, 2024.

(30) Foreign Application Priority Data
Feb. 8, 2024 (KR) 10-2024-0019342

(51) Int. Cl. C07D 473/32; A61K 31/52; A61P 1/16; A61P 9/12; A61P 11/00; A61P 25/28; A61P 29/00; A61P 35/00; A61P 37/02; C07D 487/04

(52) U.S. Cl. CPC C07D 473/32; A61K 31/52; A61P 1/16; A61P 9/12; A61P 11/00; A61P 25/28; A61P 29/00; A61P 35/00; A61P 37/02; C07D 487/04

(57) ABSTRACT

The present disclosure relates to a compound of formula (I), a stereoisomer, isotopically labeled compound, hydrate, solvate, or pharmaceutically acceptable salt thereof, a pharmaceutical composition comprising same, and a use thereof in the treatment of CXCR7-mediated diseases.

### CROSS-REFERENCE TO RELATED APPLICATIONS

This Application is a Continuation-in-Part of International Application PCT/KR2024/018655 filed on Nov. 22, 2024, which claims priority from Korean Application 10-2024-0019342 filed on Feb. 8, 2024, the entire contents of these applications are incorporated herein by reference in their entirety.

### TECHNICAL FIELD

The present disclosure relates to a novel CXCR7 (C-X-C chemokine receptor 7) receptor modulator, a pharmaceutical composition comprising the same, and a use thereof for the treatment of CXCR7-mediated diseases.

### BACKGROUND ART

Chemokine receptors, a subset of G protein-coupled receptors (GPCRs), play a pivotal role in coordinating cellular responses to chemotactic signals in various biological contexts. Among these receptors, CXCR7 (C-X-C chemokine receptor type 7), also known as ACKR3, RDC1, CMKOR1, and GPR159, is known to be involved in regulating cell migration, proliferation, and tissue regeneration.

CXCR7 has two known ligands: CXCL12 (stromal cell-derived factor 1 (SDF-1), also referred to as pre-B cell growth stimulating factor (PBSF)) and CXCL11 (interferon-inducible T cell alpha chemoattractant (I-TAC)).

CXCL12 participates in the regulation of immune surveillance and inflammatory responses. CXCL12 is secreted by bone marrow stromal cells, endothelial cells, heart, skeletal muscle, liver, brain, kidney, and parenchymal cells, and plays an essential role in stem cell proliferation, survival, and bone marrow homing of hematopoietic/precursor cells. CXCL12 also recruits bone marrow-derived precursor cells to sites of vasculature formation. It also plays a crucial role in carcinogenesis. CXCL12 promotes the recruitment of endothelial precursor cells and bone marrow-derived suppressor cells to tumor sites, as well as other bone marrow-derived cells. In addition, CXCL12 regulates angiogenesis/vasculogenesis associated with tumor progression and plays a crucial role in the dissemination of circulating tumor cells to metastatic sites. In addition to its chemotactic function, CXCL12 has been shown to regulate tumor cell proliferation, motility, and survival. In addition to CXCR7, CXCL12 binds to and activates CXCR4 (also referred to as Fusin, Leukocyte-derived seven-transmembrane-domain receptor (LESTR), D2S201E, seven-transmembrane-segment receptor, HM89, lipopolysaccharide-associated protein 3, LPS-associated protein 3), while CXCL11 binds to and activates CXCR3 (also referred to as GPR9, CD183).

Therefore, dysfunction of a series of pathways consisting of CXCR7, its ligands CXCL12 and CXCL11, and CXCR4 that binds to CXCL12 is known to be associated with various diseases related to inflammation, immune damage, immune dysfunction, tissue damage, cell growth abnormalities, and the like.

For example, indirect regulation of CXCL12 and CXCR4 through CXCR7 agonists is known to have therapeutic utility in treating diseases ranging from hepatitis to liver fibrosis (Stromal cell-derived factor-1 (SDF-1) as a target in liver diseases TC-14012, Am J Physiol Gastrointest Liver Physiol 2016).

In addition, recent studies have shown that CXCR7 participates in the Wnt/β-catenin pathway and exhibits an inhibitory effect on fibrosis (CXCR7 Inhibits Fibrosis via Wnt/β-Catenin Pathways during the Process of Angiogenesis in Human Umbilical Vein Endothelial Cells, BioMed Research International, 2020), and it has also been reported that CXCR7 agonists exhibit a protective effect on isoproterenol-induced cardiac injury (Discovery of a Novel Small-Molecule Modulator of C-X-C Chemokine Receptor Type 7 as a Treatment for Cardiac Fibrosis, J Med Chem 2018).

CXCR7 agonists have been reported to reduce the effects of LPS-induced RANKL and TNF-α-related cytokines, reduce osteoclast formation and bone resorption induced by these cytokines, and also have therapeutic effects on acute ischemic injury and thrombosis (CXC receptor 7 agonist acts as a C-X-C motif chemokine ligand 12 inhibitor to ameliorate osteoclastogenesis and bone resorption, Molecular Medicine REPORTS 2022; ACKR3 regulates platelet activation and ischemia-reperfusion tissue injury Nature Communications 2022; Discovery and Development of First-in-Class ACKR3/CXCR7 Superagonists for Platelet Degranulation Modulation, J Med Chem 2022).

Therefore, based on its potential as a therapeutic target for various diseases, the development of CXCR7 modulators that can selectively modulate CXCR7 is needed in the art.

Accordingly, the present inventors have unexpectedly discovered that novel compounds having a specific substituent combination among a group of compounds previously known as adenosine receptor antagonists (compounds disclosed in International Publication Nos. WO 2021/099837 and WO 2022/107044) exhibit excellent activity as CXCR7 modulators, thereby completing the present invention.

### DETAILED DESCRIPTION OF INVENTION

### Technical Problem

An object of the present invention is to provide a compound of Formula I below, a stereoisomer, isotopically labeled compound, hydrate, solvate, or pharmaceutically acceptable salt thereof, useful as a CXCR7 modulator.

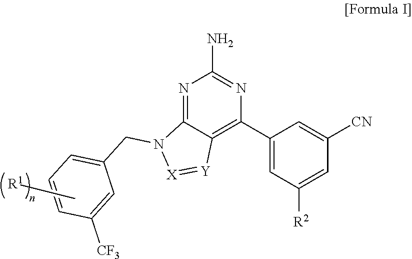

in the above formula,
X and Y are each independently CR^{a} or N;
R¹ is selected from the group consisting of hydrogen, halogen, C₁-C₆ haloalkyl, C₁-C₆ alkyl, C₁-C₆ alkoxy, C₂-C₆ alkenyl, and C₂-C₆ alkynyl;
R² is —CN or —CONR^{b}R^{c};
R^{a}, R^{b}, and R^{c} are each independently H or C₁-C₆ alkyl; and
n is an integer from 0 to 2.

Another object of the present invention is to provide a pharmaceutical composition for the treatment of CXCR7-mediated diseases, such as fibrosis, cholestatic liver disease, inflammatory disease, angiogenic disease, cancer, autoimmune disorder, osteoporosis, pulmonary hypertension, acute ischemic injury, and thrombosis.

Another object of the present invention is to provide a method for treating CXCR7-mediated diseases, comprising administering the compound of Formula I, stereoisomer, isotopically labeled compound, hydrate, solvate, or pharmaceutically acceptable salt thereof to a subject.

### Solution to Problem

Each description and embodiment disclosed in the present application may also be applied to each other description and embodiment. That is, all combinations of said various elements disclosed in the present application fall within the scope of the present application. In addition, the scope of the present application cannot be considered limited by the specific description described below.

In one aspect of the present invention, there is provided a compound of Formula I below, a stereoisomer, isotopically labeled compound, hydrate, solvate, or pharmaceutically acceptable salt thereof:

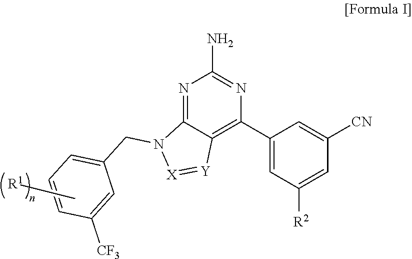

in the above formula, X and Y are each independently CR^{a} or N.

In one embodiment, X may be CR^{a}, and Y may be N. In one embodiment, X may be CR^{a}, and Y may be CR^{a}. In one embodiment, X may be N, and Y may be CR^{a}. In one embodiment, X may be N, and Y may be N. In the above definitions and embodiments, R^{a} is H or C₁-C₆ alkyl. For example, R^{a} is H or methyl.

Therefore, the compound of Formula I may be represented by any one of Formulae IA to ID below:

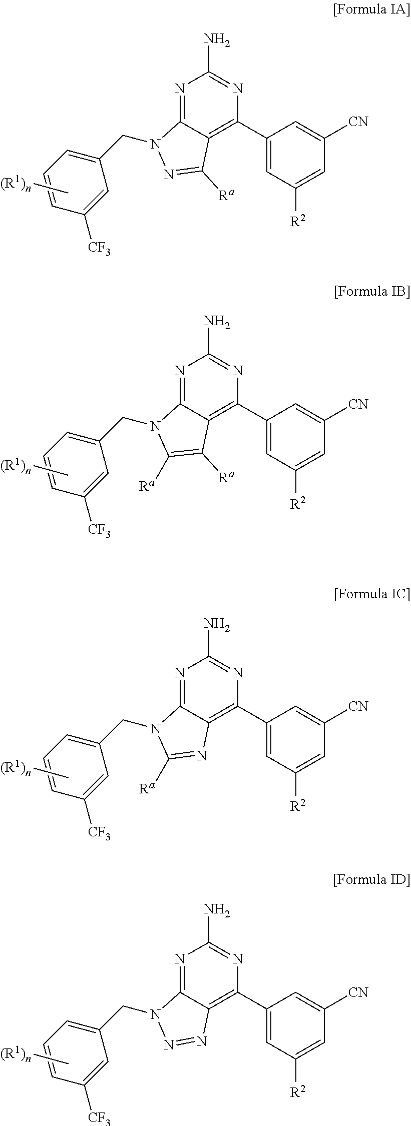

(in Formulae IA to ID above, the definitions of R¹, R², and n are the same as described below for Formula I, and the definition of R^{a} is the same as described above for Formula I.)

In Formula I and Formulae IA to ID above, R¹ is selected from the group consisting of hydrogen, halogen, C₁-C₆ haloalkyl, C₁-C₆ alkyl, C₁-C₆ alkoxy, C₂-C₆ alkenyl, and C₂-C₆ alkynyl.

In one embodiment, R¹ may be H, halogen, C₁-C₃ haloalkyl (e.g., C₁-C₃ alkyl substituted with F or Cl), C₁-C₃ alkyl, C₁-C₃ alkoxy, or C₂-C₃ alkynyl. R¹ may be H, F, Cl, Br, I, CF₃, —CH₃, —OCH₃, or —C≡C. For example, R¹ may be H, F, Cl, Br, or CF₃.

In Formula I and Formulae IA to ID above, n is an integer from 0 to 2. In one embodiment, n may be 0 or 1.

In Formula I and Formulae IA to ID above, R² is —CN or —CONR^{b}R^{c}. R^{b} and R^{c} are each independently H or C₁-C₆ alkyl.

In one embodiment, R² is —CN or —CONH₂. In one embodiment, R² is —CN. In one embodiment, R² is —CONH₂.

In one embodiment, the compound of Formula I may be represented by Formulae I-1 to I-3 below:

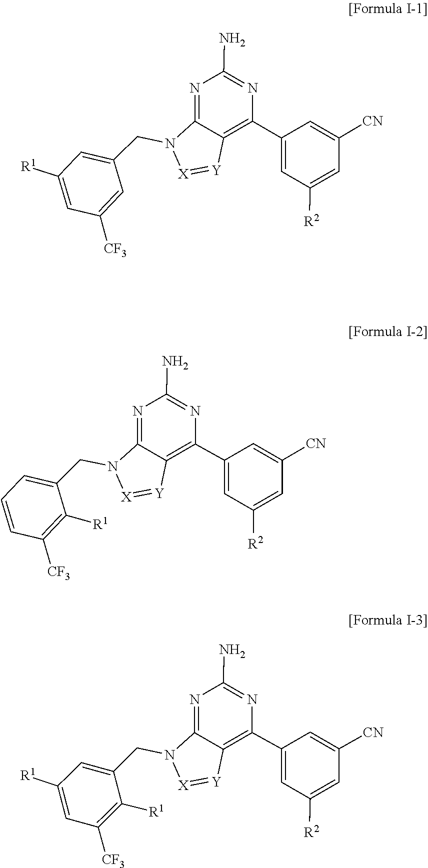

in Formulae I-1 to I-3 above, the definitions of X, Y, R¹, and R² are the same as described above for Formula I and Formulae IA to ID.

In one embodiment, in Formulae I-1 to I-3 above, R¹ may be H, halogen, or C₁-C₃ haloalkyl (e.g., C₁-C₃ alkyl substituted with F or Cl). For example, in Formulae I-1 to I-3 above, R¹ may be H, F, or C₁. In Formula I-3, two R¹ may be the same or different.

In one embodiment, in Formulae I-1 to I-3 above, R² is CN or —CONH₂.

In one embodiment, in Formulae I-1 to I-3 above, X may be N, and Y may be CR^{a}. In this case, R^{a} is H or C₁-C₆ alkyl. For example, R^{a} is H or methyl.

Meanwhile, International Publication Nos. WO 2021/099837 and WO 2022/107044 disclose that compounds containing a pyrimidine core fused to a 5-membered heterocycle are useful for treating neurodegenerative diseases such as Parkinson's disease through adenosine receptor antagonism. Based on the core disclosed in the above International Publications, the present inventors surprisingly discovered that introducing a 3-CF₃ group to the benzyl group attached to the 5-membered ring and introducing 3-CN group to the phenyl group attached to the 6-membered ring along with a CN or amide group at the 5-position, resulted in a compound that selectively targets the CXCR7 receptor without affecting the adenosine receptor, demonstrating excellent CXCR7 modulatory activity.

Therefore, the compound of Formula I according to the present invention has a characteristic structure of 3-CF₃ of a benzyl group bonded to the core and 3-CN and 5-CN or 5-CONR^{b}R^{c} of a phenyl group bonded to the core.

The compound of Formula I may be a compound selected from compounds listed in Table 1 below:

TABLE 1

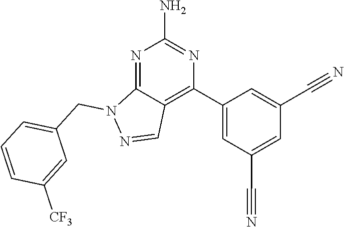 [SMILES: N#Cc1cc(C#N)cc(-c2nc(N)nc3c2cnn3Cc2cccc(C(F)(F)F)c2)c1]

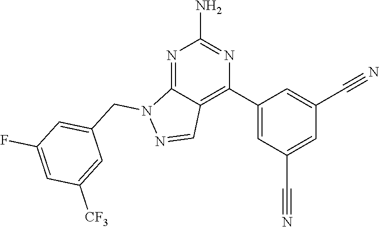 [SMILES: N#Cc1cc(C#N)cc(-c2nc(N)nc3c2cnn3Cc2cc(F)cc(C(F)(F)F)c2)c1]

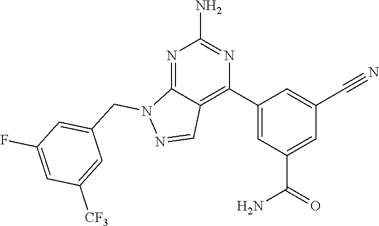 [SMILES: N#Cc1cc(C(N)=O)cc(-c2nc(N)nc3c2cnn3Cc2cc(F)cc(C(F)(F)F)c2)c1]

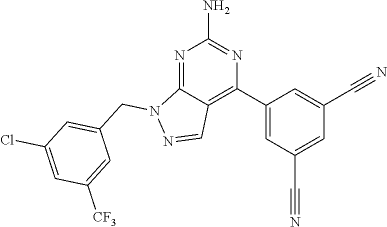 [SMILES: N#Cc1cc(C#N)cc(-c2nc(N)nc3c2cnn3Cc2cc(Cl)cc(C(F)(F)F)c2)c1]

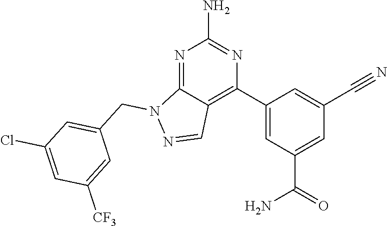 [SMILES: N#Cc1cc(C(N)=O)cc(-c2nc(N)nc3c2cnn3Cc2cc(Cl)cc(C(F)(F)F)c2)c1]

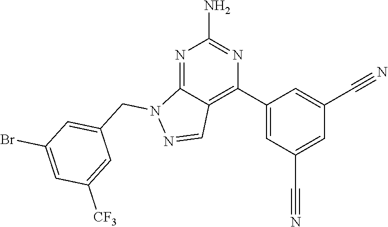 [SMILES: N#Cc1cc(C#N)cc(-c2nc(N)nc3c2cnn3Cc2cc(Br)cc(C(F)(F)F)c2)c1]

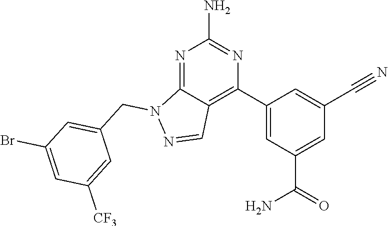 [SMILES: N#Cc1cc(C(N)=O)cc(-c2nc(N)nc3c2cnn3Cc2cc(Br)cc(C(F)(F)F)c2)c1]

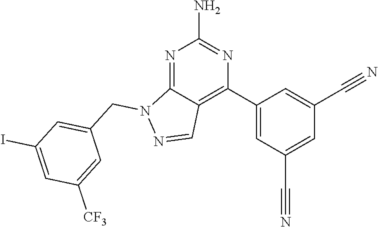 [SMILES: N#Cc1cc(C#N)cc(-c2nc(N)nc3c2cnn3Cc2cc(I)cc(C(F)(F)F)c2)c1]

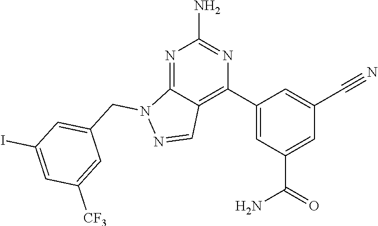 [SMILES: N#Cc1cc(C(N)=O)cc(-c2nc(N)nc3c2cnn3Cc2cc(I)cc(C(F)(F)F)c2)c1]

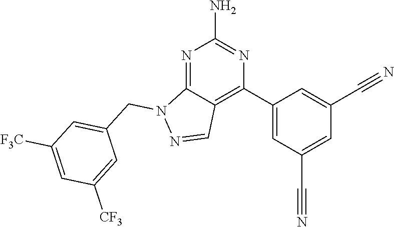 [SMILES: N#Cc1cc(C#N)cc(-c2nc(N)nc3c2cnn3Cc2cc(C(F)(F)F)cc(C(F)(F)F)c2)c1]

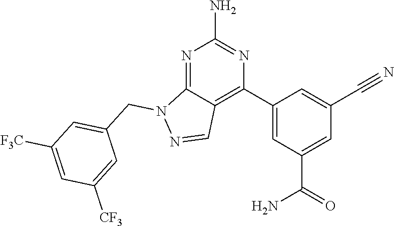 [SMILES: N#Cc1cc(C(N)=O)cc(-c2nc(N)nc3c2cnn3Cc2cc(C(F)(F)F)cc(C(F)(F)F)c2)c1]

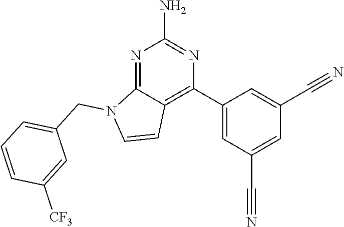 [SMILES: N#Cc1cc(C#N)cc(-c2nc(N)nc3c2ccn3Cc2cccc(C(F)(F)F)c2)c1]

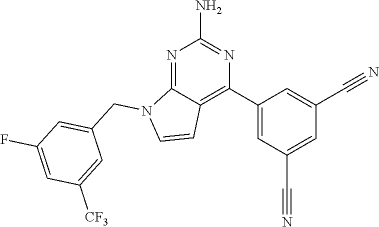 [SMILES: N#Cc1cc(C#N)cc(-c2nc(N)nc3c2ccn3Cc2cc(F)cc(C(F)(F)F)c2)c1]

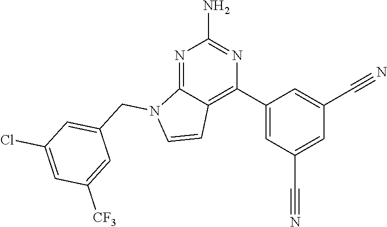 [SMILES: N#Cc1cc(C#N)cc(-c2nc(N)nc3c2ccn3Cc2cc(Cl)cc(C(F)(F)F)c2)c1]

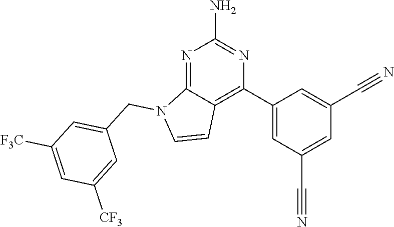 [SMILES: N#Cc1cc(C#N)cc(-c2nc(N)nc3c2ccn3Cc2cc(C(F)(F)F)cc(C(F)(F)F)c2)c1]

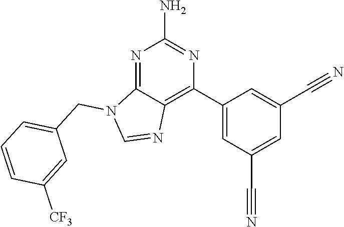 [SMILES: N#Cc1cc(C#N)cc(-c2nc(N)nc3c2ncn3Cc2cccc(C(F)(F)F)c2)c1]

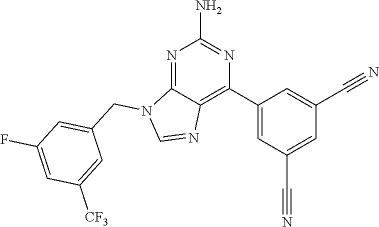 [SMILES: N#Cc1cc(C#N)cc(-c2nc(N)nc3c2ncn3Cc2cc(F)cc(C(F)(F)F)c2)c1]

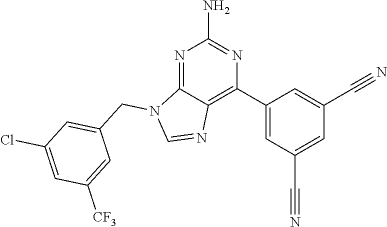 [SMILES: N#Cc1cc(C#N)cc(-c2nc(N)nc3c2ncn3Cc2cc(Cl)cc(C(F)(F)F)c2)c1]

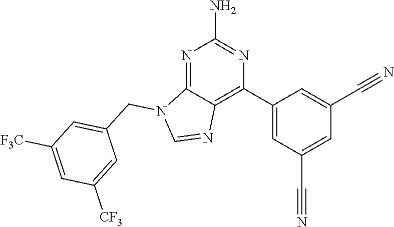 [SMILES: N#Cc1cc(C#N)cc(-c2nc(N)nc3c2ncn3Cc2cc(C(F)(F)F)cc(C(F)(F)F)c2)c1]

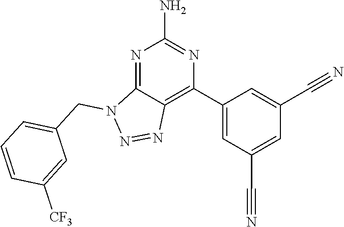 [SMILES: N#Cc1cc(C#N)cc(-c2nc(N)nc3c2nnn3Cc2cccc(C(F)(F)F)c2)c1]

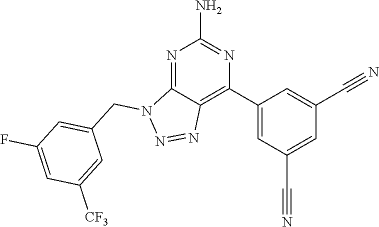 [SMILES: N#Cc1cc(C#N)cc(-c2nc(N)nc3c2nnn3Cc2cc(F)cc(C(F)(F)F)c2)c1]

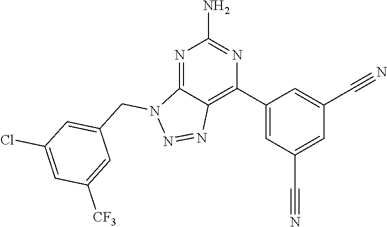 [SMILES: N#Cc1cc(C#N)cc(-c2nc(N)nc3c2nnn3Cc2cc(Cl)cc(C(F)(F)F)c2)c1]

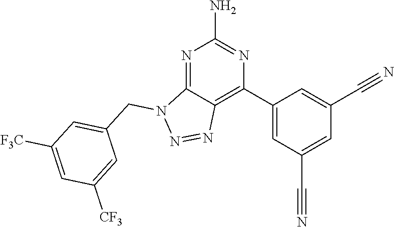 [SMILES: N#Cc1cc(C#N)cc(-c2nc(N)nc3c2nnn3Cc2cc(C(F)(F)F)cc(C(F)(F)F)c2)c1]

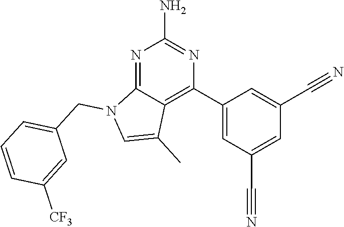 [SMILES: Cc1cn(Cc2cccc(C(F)(F)F)c2)c2nc(N)nc(-c3cc(C#N)cc(C#N)c3)c12]

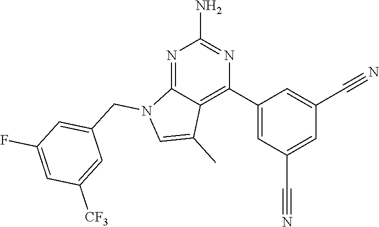 [SMILES: Cc1cn(Cc2cc(F)cc(C(F)(F)F)c2)c2nc(N)nc(-c3cc(C#N)cc(C#N)c3)c12]

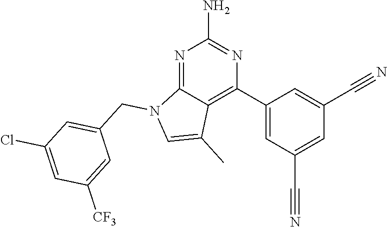 [SMILES: Cc1cn(Cc2cc(Cl)cc(C(F)(F)F)c2)c2nc(N)nc(-c3cc(C#N)cc(C#N)c3)c12]

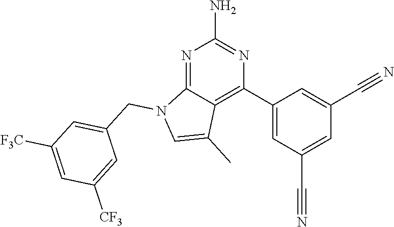 [SMILES: Cc1cn(Cc2cc(C(F)(F)F)cc(C(F)(F)F)c2)c2nc(N)nc(-c3cc(C#N)cc(C#N)c3)c12]

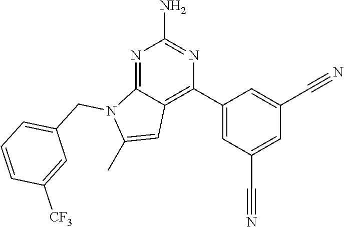 [SMILES: Cc1cc2c(-c3cc(C#N)cc(C#N)c3)nc(N)nc2n1Cc1cccc(C(F)(F)F)c1]

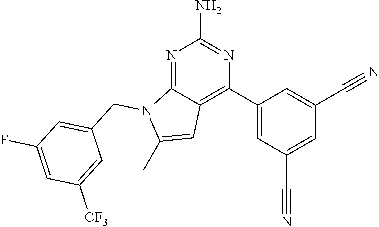 [SMILES: Cc1cc2c(-c3cc(C#N)cc(C#N)c3)nc(N)nc2n1Cc1cc(F)cc(C(F)(F)F)c1]

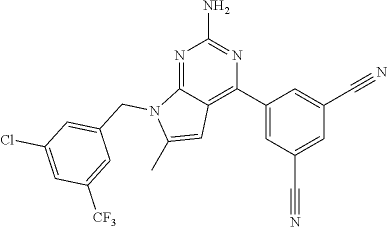 [SMILES: Cc1cc2c(-c3cc(C#N)cc(C#N)c3)nc(N)nc2n1Cc1cc(Cl)cc(C(F)(F)F)c1]

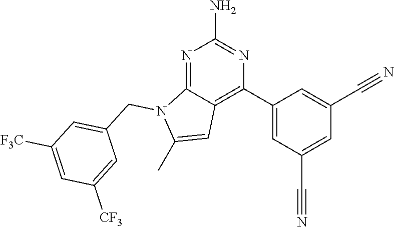 [SMILES: Cc1cc2c(-c3cc(C#N)cc(C#N)c3)nc(N)nc2n1Cc1cc(C(F)(F)F)cc(C(F)(F)F)c1]

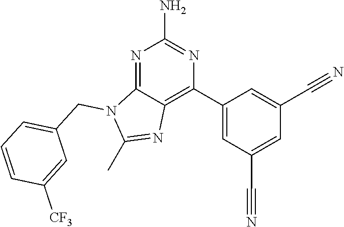 [SMILES: Cc1nc2c(-c3cc(C#N)cc(C#N)c3)nc(N)nc2n1Cc1cccc(C(F)(F)F)c1]

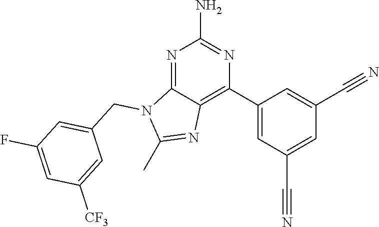 [SMILES: Cc1nc2c(-c3cc(C#N)cc(C#N)c3)nc(N)nc2n1Cc1cc(F)cc(C(F)(F)F)c1]

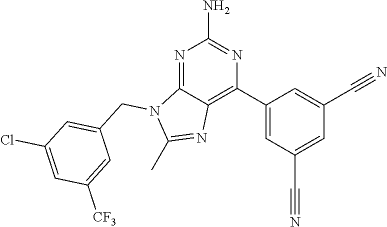 [SMILES: Cc1nc2c(-c3cc(C#N)cc(C#N)c3)nc(N)nc2n1Cc1cc(Cl)cc(C(F)(F)F)c1]

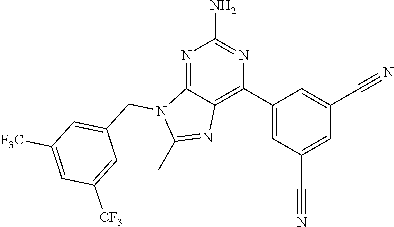 [SMILES: Cc1nc2c(-c3cc(C#N)cc(C#N)c3)nc(N)nc2n1Cc1cc(C(F)(F)F)cc(C(F)(F)F)c1]

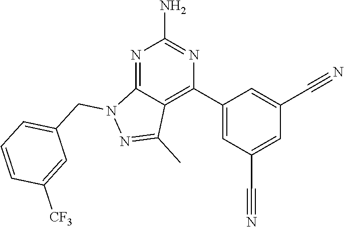 [SMILES: Cc1nn(Cc2cccc(C(F)(F)F)c2)c2nc(N)nc(-c3cc(C#N)cc(C#N)c3)c12]

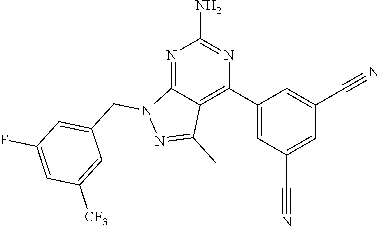 [SMILES: Cc1nn(Cc2cc(F)cc(C(F)(F)F)c2)c2nc(N)nc(-c3cc(C#N)cc(C#N)c3)c12]

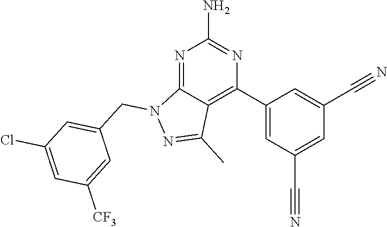 [SMILES: Cc1nn(Cc2cc(Cl)cc(C(F)(F)F)c2)c2nc(N)nc(-c3cc(C#N)cc(C#N)c3)c12]

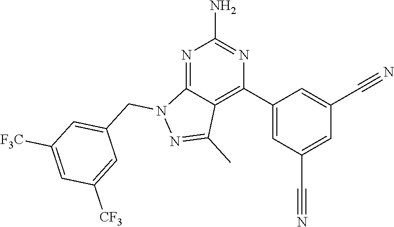 [SMILES: Cc1nn(Cc2cc(C(F)(F)F)cc(C(F)(F)F)c2)c2nc(N)nc(-c3cc(C#N)cc(C#N)c3)c12]

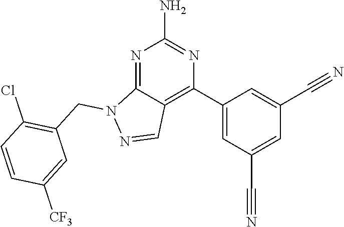 [SMILES: N#Cc1cc(C#N)cc(-c2nc(N)nc3c2cnn3Cc2cc(C(F)(F)F)ccc2Cl)c1]

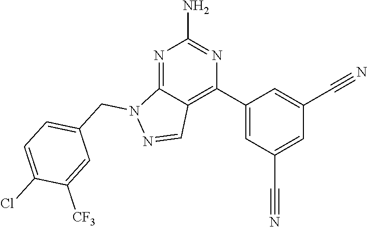 [SMILES: N#Cc1cc(C#N)cc(-c2nc(N)nc3c2cnn3Cc2ccc(Cl)c(C(F)(F)F)c2)c1]

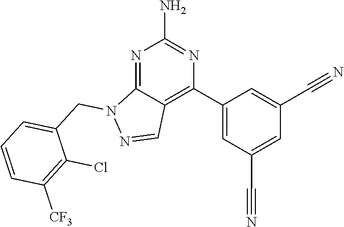 [SMILES: N#Cc1cc(C#N)cc(-c2nc(N)nc3c2cnn3Cc2cccc(C(F)(F)F)c2Cl)c1]

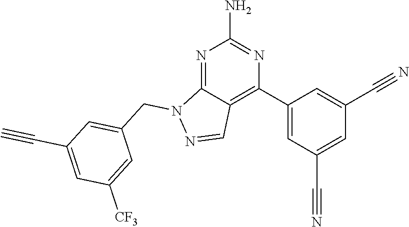 [SMILES: C#Cc1cc(Cn2ncc3c(-c4cc(C#N)cc(C#N)c4)nc(N)nc32)cc(C(F)(F)F)c1]

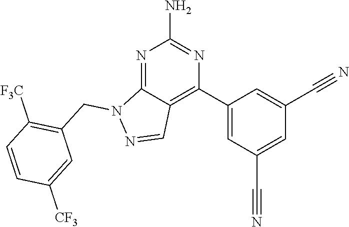 [SMILES: N#Cc1cc(C#N)cc(-c2nc(N)nc3c2cnn3Cc2cc(C(F)(F)F)ccc2C(F)(F)F)c1]

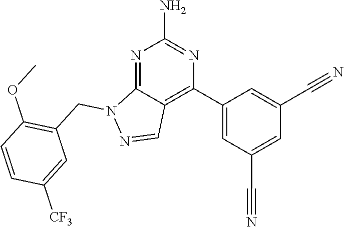 [SMILES: COc1ccc(C(F)(F)F)cc1Cn1ncc2c(-c3cc(C#N)cc(C#N)c3)nc(N)nc21]

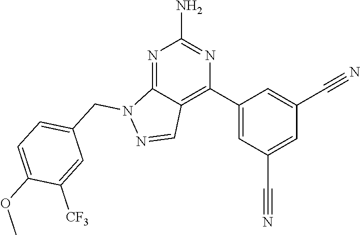 [SMILES: COc1ccc(Cn2ncc3c(-c4cc(C#N)cc(C#N)c4)nc(N)nc32)cc1C(F)(F)F]

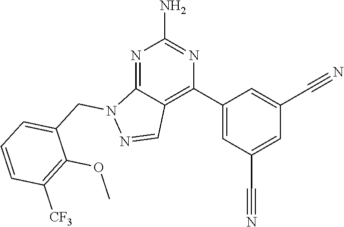 [SMILES: COc1c(Cn2ncc3c(-c4cc(C#N)cc(C#N)c4)nc(N)nc32)cccc1C(F)(F)F]

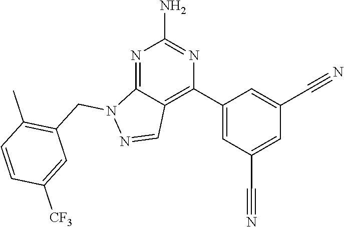 [SMILES: Cc1ccc(C(F)(F)F)cc1Cn1ncc2c(-c3cc(C#N)cc(C#N)c3)nc(N)nc21]

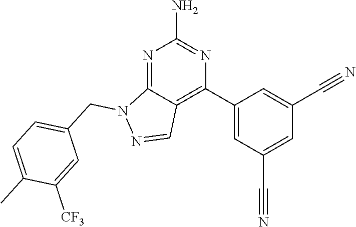 [SMILES: Cc1ccc(Cn2ncc3c(-c4cc(C#N)cc(C#N)c4)nc(N)nc32)cc1C(F)(F)F]

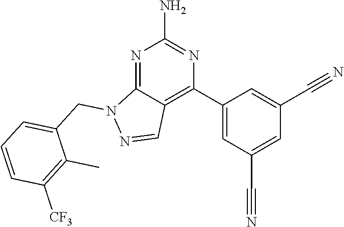 [SMILES: Cc1c(Cn2ncc3c(-c4cc(C#N)cc(C#N)c4)nc(N)nc32)cccc1C(F)(F)F]

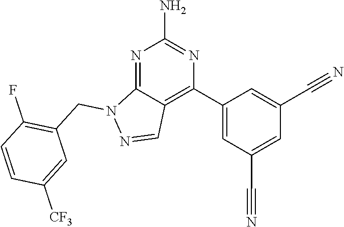 [SMILES: N#Cc1cc(C#N)cc(-c2nc(N)nc3c2cnn3Cc2cc(C(F)(F)F)ccc2F)c1]

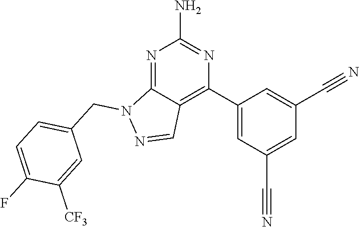 [SMILES: N#Cc1cc(C#N)cc(-c2nc(N)nc3c2cnn3Cc2ccc(F)c(C(F)(F)F)c2)c1]

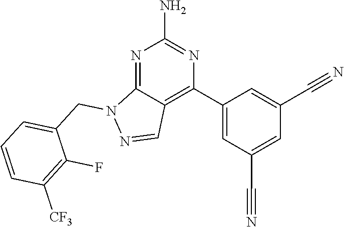 [SMILES: N#Cc1cc(C#N)cc(-c2nc(N)nc3c2cnn3Cc2cccc(C(F)(F)F)c2F)c1]

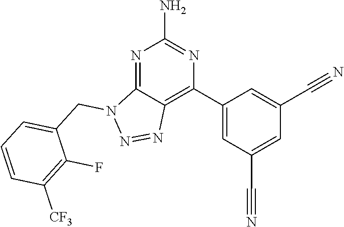 [SMILES: N#Cc1cc(C#N)cc(-c2nc(N)nc3c2nnn3Cc2cccc(C(F)(F)F)c2F)c1]

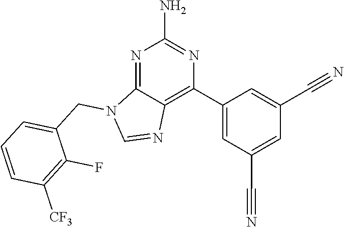 [SMILES: N#Cc1cc(C#N)cc(-c2nc(N)nc3c2ncn3Cc2cccc(C(F)(F)F)c2F)c1]

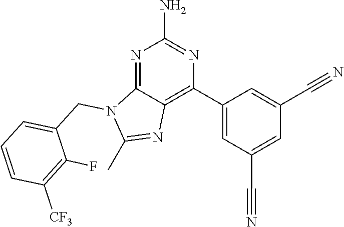 [SMILES: Cc1nc2c(-c3cc(C#N)cc(C#N)c3)nc(N)nc2n1Cc1cccc(C(F)(F)F)c1F]

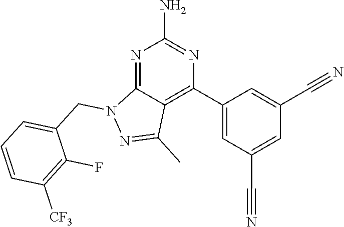 [SMILES: Cc1nn(Cc2cccc(C(F)(F)F)c2F)c2nc(N)nc(-c3cc(C#N)cc(C#N)c3)c12]

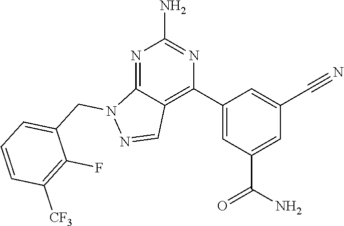 [SMILES: N#Cc1cc(C(N)=O)cc(-c2nc(N)nc3c2cnn3Cc2cccc(C(F)(F)F)c2F)c1]

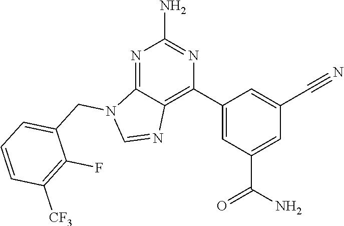 [SMILES: N#Cc1cc(C(N)=O)cc(-c2nc(N)nc3c2ncn3Cc2cccc(C(F)(F)F)c2F)c1]

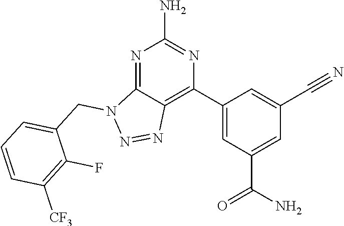 [SMILES: N#Cc1cc(C(N)=O)cc(-c2nc(N)nc3c2nnn3Cc2cccc(C(F)(F)F)c2F)c1]

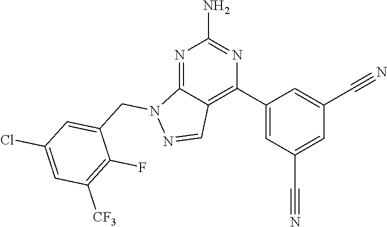 [SMILES: N#Cc1cc(C#N)cc(-c2nc(N)nc3c2cnn3Cc2cc(Cl)cc(C(F)(F)F)c2F)c1]

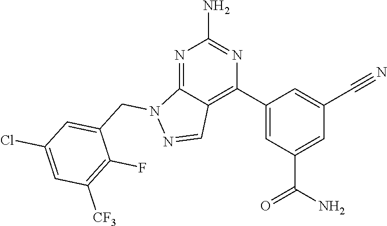 [SMILES: N#Cc1cc(C(N)=O)cc(-c2nc(N)nc3c2cnn3Cc2cc(Cl)cc(C(F)(F)F)c2F)c1]

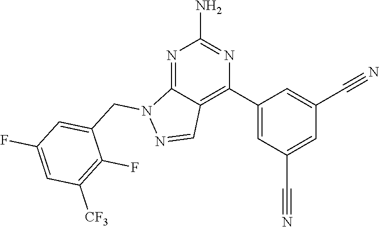 [SMILES: N#Cc1cc(C#N)cc(-c2nc(N)nc3c2cnn3Cc2cc(F)cc(C(F)(F)F)c2F)c1]

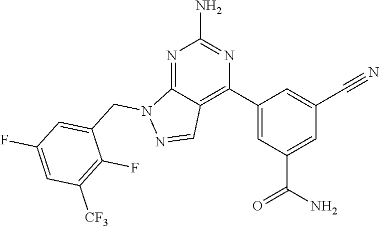 [SMILES: N#Cc1cc(C(N)=O)cc(-c2nc(N)nc3c2cnn3Cc2cc(F)cc(C(F)(F)F)c2F)c1]

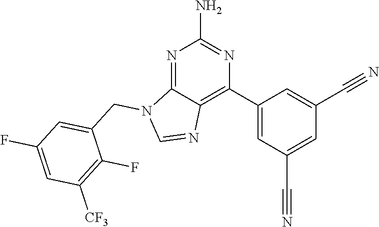 [SMILES: N#Cc1cc(C#N)cc(-c2nc(N)nc3c2ncn3Cc2cc(F)cc(C(F)(F)F)c2F)c1]

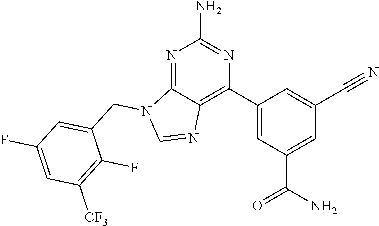 [SMILES: N#Cc1cc(C(N)=O)cc(-c2nc(N)nc3c2ncn3Cc2cc(F)cc(C(F)(F)F)c2F)c1]

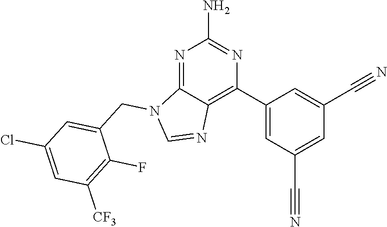 [SMILES: N#Cc1cc(C#N)cc(-c2nc(N)nc3c2ncn3Cc2cc(Cl)cc(C(F)(F)F)c2F)c1]

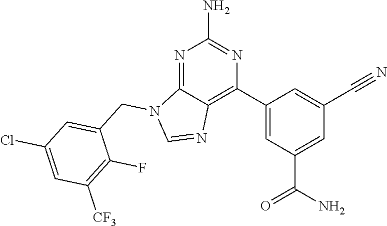 [SMILES: N#Cc1cc(C(N)=O)cc(-c2nc(N)nc3c2ncn3Cc2cc(Cl)cc(C(F)(F)F)c2F)c1]

The compounds of the present invention, including stereoisomers, isotopically labeled compounds, hydrates, solvates, and salts, can be prepared by known organic synthetic methods and can be synthesized via a number of synthetic routes.

The reaction for preparing the compound of the present invention can be performed in a suitable solvent that can be appropriately selected by those of ordinary skill in the art of organic synthesis. Suitable solvents are those that are substantially non-reactive with the starting materials (reactants), intermediates, or target products at the temperature at which the reaction occurs. Those of ordinary skill in the art will be able to appropriately select a suitable solvent for each reaction step.

Protection and deprotection of various functional groups can occur during the synthesis of the compound of the present invention. Those of ordinary skill in the art will readily be able to determine the necessity of protection and deprotection and select appropriate protecting groups.

Each reaction can be monitored by any suitable method known in the art. For example, the synthesis of the desired compound can be monitored by spectroscopic means, such as NMR (e.g., 1H or 13C), mass spectroscopy, or chromatography (HPLC or TLC).

If necessary, the compound of the present invention can be purified by, for example, chromatography, crystallization from a solvent or solvent mixture, distillation, extraction, or the like. Chromatography can include, but is not limited to, reverse-phase, normal phase, size exclusion, ion exchange, preparative, or flash chromatography. Those of ordinary skill in the art will readily be able to select the optimal technique for purifying the target product.

If the compound of the present invention is a stereoisomer, it can be isolated from the racemic mixture by any suitable method, if necessary. Examples include separation by forming ionic or diastereomeric salts using chiral compounds and fractional crystallization, formation and separation of diastereoisomers using chiral derivatizing reagents, followed by conversion to pure stereoisomers, and direct separation of substantially pure stereoisomers under chiral conditions.

The compound of the present invention can be synthesized according to the synthetic procedures described in the examples below. Based on the above, the desired compound can be prepared by appropriately changing the reactants and reaction conditions, etc., depending on the structure of the desired compound.

Specifically, the compound of Formula I according to the present invention can be prepared by introducing an appropriate substituent corresponding to the structure of the desired compound into an intermediate having a pyrazolopyrimidine core (e.g., Intermediate S1, Intermediate S2, or Intermediate S3), a pyrrolopyrimidine core (e.g., Intermediate S4), a purine core (e.g., Intermediate S5), or a triazolopyrimidine core (e.g., Intermediate S6).

Methods for preparing intermediates having each of the above cores and exemplary compounds having appropriate substituents introduced therein according to the present invention are described in detail in the following Preparation Examples. As an example, the compound of the present invention having a pyrazolopyrimidine core can be prepared according to Reaction Scheme I below.

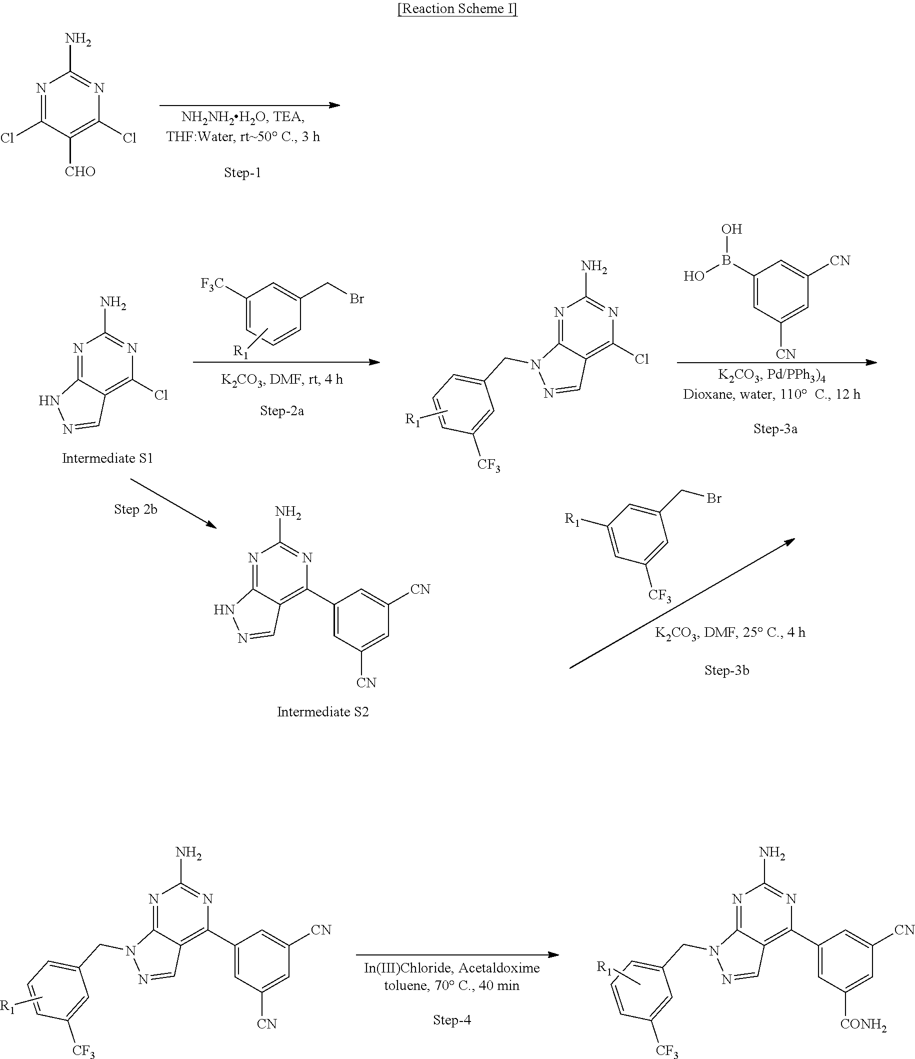

According to Reaction Scheme I above, the compound of the present invention having a pyrazolopyrimidine core can be prepared through Steps 1 to 4 below:
Step 1: Dissolving 2-amino-4,6-dichloropyrimidine-5-carbaldehyde in a solvent of tetrahydrofuran and H₂O, and subjecting the solution to a chain reaction with hydrazine monohydrate at 50° C. for 3 hours to prepare Intermediate S1.
Step 2a: Dissolving Intermediate S1 in the N,N dimethylformamide solvent, and then adding potassium carbonate or cesium carbonate to cause a nucleophilic substitution reaction with a benzene bromide compound substituted with R¹ and CF₃ at room temperature for 4 hours.
Step 3a: Dissolving the product of Step 2a in 1,4-dioxane and distilled water, and then subjecting the solution to a Suzuki coupling reaction with 5-(4,4,5,5-tetramethyl-1,3,2-dioxaborolan-2-yl)isophthalonitrile at 110° C. for 12 hours to prepare a compound of Formula I having 3,5-CN substituted on the phenyl group.
Step 4: If necessary, further reacting at 70° C. for 40 minutes under the conditions of InCl₃ and acetaldoxime through monocyano hydrolysis to prepare a compound of Formula I having 3-CN and 5-amide substituted on the phenyl group.

Alternatively, Steps 2a and 3a can be performed in reverse order, as Steps 2b and 3b of the above reaction scheme. The above reaction conditions, reaction times, reagents, and the like are merely examples. Those of ordinary skill in the art will be able to appropriately modify and prepare the compound of the present invention based on the disclosures of the present specification, including the examples herein.

### Definition

All technical and scientific terms used herein have meanings commonly understood by those of ordinary skill in the art. Unless otherwise stated, conventional measurement methods, manufacturing methods, and conventional ingredients or materials are used based on conventional techniques such as pharmacology, pharmaceutical chemistry, mass spectrometry, NMR, HPLC, and biochemistry.

The individual features and components of each embodiment described and exemplified herein may be combined with the features and components of any other embodiment without departing from the scope or spirit of the present disclosure.

Unless otherwise specified, in the present specification and the appended claims, “and” and “or” mean “and/or.” The terms “include” and “included” are open-ended and mean that a compound, composition, or method may include additional features or components in addition to the specific features or components listed.

As used herein, the numerical range indicated using the term “to” refers to a range that includes the numerical values described before and after the term “to” as the lower limit and the upper limit, respectively.

As used herein, the term “optional” or “optionally” means that a subsequently described event or circumstance may or may not occur, and that the description includes instances in which said event or circumstance occurs and instances in which it does not occur. For example, the term “optionally substituted” includes instances where the element is substituted or unsubstituted with the specified substituent.

### Compound

The term “halogen” refers to an atom belonging to Group 17 of the Periodic Table. Halogen atoms include fluorine (F), chlorine (Cl), bromine (Br), and iodine (I). The term “halogen” can be used interchangeably with the term “halo,” which refers to a monovalent functional group composed of a halogen.

The term “hydroxy” refers to the —OH functional group (hydroxyl group).

The term “—CN” or “cyano” refers to a functional group composed of a triple bond between a carbon atom and a nitrogen atom.

The term “amino” refers to a functional group in which hydrogen is bonded to a nitrogen atom, i.e., —NH₂.

The term “alkyl” refers to a fully saturated, branched or unbranched (or straight-chain or linear) hydrocarbon. The alkyl group may be a substituted or unsubstituted alkyl group. The alkyl group may be a C₁ to C₆ alkyl group, a C₁ to C₅ alkyl group, a C₁ to C₄ alkyl group, a C₁ to C₃ alkyl group, or a C₁ to C₂ alkyl group. Non-limiting examples of the alkyl group may include methyl, ethyl, n-propyl, isopropyl, n-butyl, isobutyl, sec-butyl, n-pentyl, isopentyl, neopentyl, iso-amyl, or n-hexyl.

The term “haloalkyl” refers to a straight-chain or branched saturated aliphatic hydrocarbyl group having a specified number of carbon atoms, substituted with one or more halogen atoms. The haloalkyl group includes a perhaloalkyl group, in which all hydrogens of the alkyl group are substituted with halogens (e.g., —CF₃, —CF₂CF₃). The above halogens may be the same (e.g., CHF₂, —CF₃) or different (e.g., CF₂Cl). If specified, the haloalkyl group may be optionally substituted with one or more substituents other than the halogen. Examples of the haloalkyl group include, but are not limited to, fluoromethyl, dichloroethyl, trifluoromethyl, trichloromethyl, pentafluoroethyl, and pentachloroethyl groups.

As used herein, the term “alkylene” refers to a divalent, fully saturated, branched or unbranched (or straight-chain or linear) hydrocarbyl having a functional group represented by the formula —CₙH₂ₙ—. For example, C₁-C₆ alkylene may include ethylene, propylene, butylene, and hexylene.

The term “alkenyl” refers to a linear or branched hydrocarbyl group having 2 to 6 carbon atoms, 2 to 4 carbon atoms, or 2 to 3 carbon atoms having at least one vinyl unsaturation site (>C═C<). For example, (Cₓ-C_{y}) alkenyl refers to an alkenyl group having x to y carbon atoms, and may include, for example, ethenyl, propenyl, isopropylene, 1,3-butadienyl, and the like.

The term “alkynyl” refers to a linear or branched monovalent hydrocarbyl radical containing at least one triple bond. The term “alkynyl” may also include a hydrocarbyl group having one triple bond and one double bond. For example, (C₂-C₆) alkynyl may include ethynyl, propynyl, and the like.

The term “alkoxy” refers to a substituent in which a substituted or unsubstituted straight-chain or branched alkyl moiety is linked to another chemical structure via oxygen. The alkoxy may include, without limitation, all possible isomers thereof, such as methoxy, ethoxy, propoxy, and butoxy, or isopropoxy, isobutoxy, and t-butoxy.

The term “substitution” in the above “optionally substituted” refers to introducing a substitute for the hydrogen atom in the case where a derivative is formed by substituting one or more hydrogen atoms in an organic compound with another atomic group, and “substituent” refers to the introduced atomic group. As used herein, the term “substituted” group refers to one in which one or more hydrogen atoms are replaced with one or more non-hydrogen atom groups, provided that valence requirements should be met and a chemically stable compound should occur from the substitution. In the present specification, unless explicitly stated as “unsubstituted,” all substituents should be construed as being capable of being unsubstituted or substituted.

In the present specification, when a combination of substituents is mentioned such as one group, for example, haloalkyl, hydroxyalkyl, or the like, the last-mentioned group generally contains the atom attached to the end of the molecule. As used herein, “ [symbol] ”, or “—” is used to indicate a position at which a substituent is bonded to the moieties of the compound. For example, if “—” is indicated at the end of a substituent, it means that the end is attached to the remaining moieties of the compound. In addition, when two or more substituents are linked by “—,” it means that the substituent immediately before “—” is bonded to a substitutable atom of the substituent immediately after “—.”

As used herein, the term “isotope” refers to an element that is the same element but has a different mass number, i.e., an element with the same number of protons but a different number of neutrons. Examples include, but are not limited to, ²H, ³H, ¹¹C, ¹³C, ¹⁴C, ¹⁵N, ¹⁸F, ³¹P, ³²P, ³⁵S, ³⁶Cl, ¹²⁵I, and the like. Isotopically labeled compounds may be advantageous in terms of improved stability in the body and a longer half-life.

As used herein, the term “solvate” may refer to a compound of the present invention or a salt thereof comprising a stoichiometric or non-stoichiometric amount of a solvent bound by non-covalent intermolecular forces. Preferred solvents therefor may be solvents that are volatile, non-toxic, and/or suitable for administration to humans. Those of ordinary skill in the art will be able to readily prepare a solvate, such as a hydrate, of the compound disclosed herein using appropriate techniques known in the art.

As used herein, the term “stereoisomer” may refer to a compound of the present invention or a salt thereof that has the same chemical formula or molecular formula but is optically or sterically different, and specifically, may be a diastereomer, an enantiomer, or a geometric isomer.

In some embodiments, the compound of the present invention contains one or more asymmetric centers and may be in the form of a racemate, a single enantiomer, a mixture of enantiomers, a single diastereomer, a mixture of diastereomers, etc. In one embodiment, due to the nature of the asymmetric center or limited rotation, the compound of the present invention may exist in the form of an enantiomer or a diastereomer.

When two or more asymmetric centers are present in the compound of the present invention, multiple diastereomers and enantiomers of the chemical structures disclosed herein may exist. Pure isomers, separated isomers, partially pure isomers, or racemic mixtures are all intended to be within the scope of the present invention.

Purification of the isomers and separation of isomer mixtures can be achieved by standard techniques known in the art. For example, a diastereomeric mixture can be separated into its respective diastereomers by a chromatographic process or crystallization, and a racemate can be separated into its respective enantiomers by a chromatographic process or resolution of the chiral phase.

The term “salt” refers to inorganic and organic acid addition salts of a compound. The compound of the present invention can be used in the form of a pharmaceutically acceptable salt derived from inorganic or organic acids. The pharmaceutically acceptable salt may be a salt that does not cause significant irritation to the organism to which the compound is administered and do not impair the biological activity and physical properties of the compound. The inorganic acid salts may be hydrochloride, bromate, phosphate, sulfate, or disulfate. The organic acid salt may be formate, acetate, propionate, lactate, oxalate, tartrate, malate, maleate, citrate, fumarate, besylate, camsylate, edisylate, trichloroacetic acid, trifluoroacetate, benzoate, gluconate, methanesulfonate, glycolate, succinate, 4-toluenesulfonate, galacturonate, embonate, glutamate, ethanesulfonate, benzenesulfonate, p-toluenesulfonate, or aspartate. In addition, the metal salt may be a calcium salt, sodium salt, magnesium salt, strontium salt, or potassium salt.

A pharmaceutically acceptable salt of the compound according to the present invention may be prepared by dissolving the compound of Formula I in a water-miscible organic solvent, such as acetone, methanol, ethanol, acetonitrile, or the like, and adding an excess of an organic acid or adding an aqueous acid solution of an inorganic acid, and then precipitating or crystallizing. Subsequently, after evaporating the solvent or an excess of acid from this mixture, it may be prepared by drying to obtain an addition salt or by suction filtration of the precipitated salt.

Medicinal Use, Pharmaceutical Composition, and Administration Method In another aspect, there is provided a pharmaceutical composition comprising a compound, a stereoisomer, isotopically labeled compound, hydrate, solvate, or pharmaceutically acceptable salt thereof according to one aspect. The compound, the stereoisomer, the isotopically labeled compound, the solvate, and the salt are the same as described above.

The compound, a stereoisomer, isotopically labeled compound, hydrate, solvate, or pharmaceutically acceptable salt thereof according to one aspect of the present invention selectively acts on the CXCR7 receptor and is useful for the treatment of CXCR7-mediated diseases.

The compound, a stereoisomer, isotopically labeled compound, hydrate, solvate, or pharmaceutically acceptable salt thereof according to the present invention selectively activate CXCR7 and its downstream signaling pathways, providing high specificity for CXCR7, improved pharmacokinetic properties, reduced off-target effects, and enhanced efficacy in modulating cellular responses associated with CXCR7 activation. As used herein, a “CXCR7-mediated disease” may be a disease caused by dysfunction in a series of pathways consisting of CXCR7, its ligands CXCL12 and CXCL11, and CXCR4, which binds to CXCL12. For example, the compound according to the present invention may exhibit therapeutic effects on various diseases by counteracting the effects of CXCL12 through the activation of CXCR7.

The CXCR7-mediated diseases include fibrosis, cholestatic liver disease, inflammatory disease, angiogenic disease, cancer, autoimmune disorder, osteoporosis, pulmonary hypertension, acute ischemic injury, and thrombosis.

In one embodiment, the compound, a stereoisomer, isotopically labeled compound, hydrate, solvate, or pharmaceutically acceptable salt thereof according to the present invention may be useful for the treatment of fibrosis through selective modulatory activity against CXCR7.

In one embodiment, the fibrosis includes fibrosis of the liver, lung, skin, kidney, heart, joint, or intestine.

In one embodiment, the fibrosis may be selected from the group consisting of liver fibrosis, liver cirrhosis, pulmonary fibrosis, idiopathic pulmonary fibrosis, interstitial lung disease, kidney fibrosis, cardiac fibrosis, and arthrofibrosis. For example, the fibrosis may be liver fibrosis, pulmonary fibrosis, idiopathic pulmonary fibrosis, or cardiac fibrosis.

Fibrosis refers to a pathological process in which excessive accumulation of fibrous connective tissue leads to the formation of scar tissue within an organ or tissue. This abnormal tissue formation disrupts the normal structure and function of the affected organ, affecting a wide range of organs, including the lungs (pulmonary fibrosis), liver (liver cirrhosis), heart (cardiac fibrosis), kidneys, and skin.

CXCR7 has been reported to have an antifibrotic effect by participating in the Wnt/β-catenin pathway, and this effect is particularly evident during angiogenesis (CXCR7 Inhibits Fibrosis via Wnt/β-Catenin Pathways during the Process of Angiogenesis in Human Umbilical Vein Endothelial Cells, BioMed Research International, 2020).

Overexpression of CXCR7 has been observed to inhibit fibrosis via the Wnt/β-catenin pathway during angiogenesis in human umbilical vein endothelial cells (HUVECs), suggesting that CXCR7 effectively influences the vascular fibrosis switch in the pathophysiology of angiogenesis.

A significant antifibrotic effect of CXCR7 agonists has been reported in a carbon tetrachloride (CCl₄)-induced liver fibrosis model (Divergent angiocrine signals from vascular niche balance liver regeneration and fibrosis, Nature 2014; Stimulation of the atypical chemokine receptor 3 (ACKR3) by a small-molecule agonist attenuates fibrosis in a preclinical liver but not lung injury model, Cell Mol Life Sci 2022).

In addition, a study reported that a selective CXCR7 agonist significantly reduced isoproterenol-induced cardiac fibrosis (Discovery of a Novel Small-Molecule Modulator of C-X-C Chemokine Receptor Type 7 as a Treatment for Cardiac Fibrosis, J Med Chem 2018).

Without being bound by theory, CXCR7 is thought to play the following roles, for example, in fibrosis:
(1) Fibroblast activation and proliferation: Activation of CXCR7 in endothelial cells may reduce EndMT and α-SMA expression, thereby attenuating fibrotic responses.
(2) Angiogenesis and tissue remodeling: CXCR7 influences angiogenesis, which may significantly impact tissue regeneration.

The present inventors experimentally demonstrated that the compound according to the present invention significantly reduces fibrosis in idiopathic pulmonary fibrosis and liver fibrosis models.

In one embodiment, the compound, a stereoisomer, isotopically labeled compound, hydrate, solvate, or pharmaceutically acceptable salt thereof according to the present invention may be useful for the treatment of cholestatic liver disease through its selective modulatory activity against CXCR7. Cholestatic liver disease is a collective term for biochemical, physiological, and clinical changes resulting from impaired circulation of bile produced in the liver and circulating through the biliary tract and intestines. For example, the cholestatic liver disease may include primary biliary cholangitis (PBC), primary sclerosing cholangitis (PSC), cholesterol gallstones, intrahepatic cholestasis of pregnancy, progressive familial intrahepatic cholestasis (PFIC), or Alagille syndrome, but is not limited thereto.

CXCR7 can exert significant therapeutic effects in various conditions primarily through mechanisms such as inhibition of hepatic inflammation, inhibition of fibrosis, cytoprotection, and regulation of cholesterol metabolism. For example, CXCR7 activation inhibited fibrosis in an environment of cholestatic liver injury due to bile duct ligation, which is an animal model of primary biliary cholangitis (PBC), an autoimmune liver disease (Divergent angiocrine signals from vascular niche balance liver regeneration and fibrosis, Nature 2014). This CXCR7 activation exhibits a dual action of fundamentally blocking pathological fibrosis, thereby promoting the functional recovery of damaged liver tissue. In terms of preventing cholesterol gallstones, CXCR7 agonists have been reported to regulate cholesterol metabolism and excretion in the liver and affect bile acid metabolism, thereby inhibiting gallstone formation (Activation of CXCR7 Limits Atherosclerosis and Improves Hyperlipidemia by Increasing Cholesterol Uptake in Adipose Tissue, Circulation, 129, 1244-1253). Therefore, CXCR7 agonists can provide an important therapeutic approach for cholestatic liver disease.

In particular, the present inventors experimentally confirmed that the compound of the present invention significantly reduced ductal proliferation, portal inflammation, and confluent necrosis, and significantly inhibited liver tissue fibrosis in a cholestatic liver disease model induced by bile duct ligation.

In one embodiment, the compound, a stereoisomer, isotopically labeled compound, hydrate, solvate, or pharmaceutically acceptable salt thereof according to the present invention may be useful for the treatment of inflammatory diseases through selective modulatory activity against CXCR7. Examples of such inflammatory diseases may include arthritis, asthma, chronic obstructive pulmonary disease, atherosclerosis, myocarditis, sarcoidosis, or sinusitis, but are not limited thereto.

CXCR7 agonists have been reported to reduce the effects of LPS-induced RANKL and TNF-α-related cytokines (CXC receptor 7 agonist acts as a C-X-C motif chemokine ligand 12 inhibitor to ameliorate osteoclastogenesis and bone resorption, Molecular Medicine REPORTS 2022, 25(3), 78). Therefore, the compound of the present invention exhibiting selective modulatory activity against CXCR7 may be useful for the treatment of diseases caused by persistent inflammatory responses, such as arthritis.

In one embodiment, the compound, a stereoisomer, isotopically labeled compound, hydrate, solvate, or pharmaceutically acceptable salt thereof according to the present invention may be useful for the treatment of angiogenic diseases through selective modulatory activity against CXCR7. For example, angiogenic diseases may include rheumatoid arthritis, psoriasis, diabetic retinopathy, retinopathy of prematurity, decreased visual acuity, corneal graft rejection, neovascular glaucoma, retrolental fibroplasia, skin flushing, Osler-Weber syndrome, myocardial neovascularization, plaque neovascularization, telangiectasia, hemophilic joint, angiofibroma, intestinal adhesions, Crohn's disease, eczema, scleroderma, diabetes, atherosclerosis, wound granulation, or keloid, but are not limited thereto.

Previous studies have shown that activation of CXCR7 inhibits CXCL12-induced HUVEC tube formation and reduces CXCR4 protein levels. These study results suggest that when CXCR7 expression is increased due to inflammation, CXCR7 dimerizes with CXCR4, inducing its internalization and degradation, thereby acting as a negative regulator of CXCR4. Therefore, it was proposed that CXCR7 activation could counteract the effects of CXCL12, thereby exerting therapeutic effects against CXCL12-induced diseases (CXCR7 agonists inhibit the function of CXCL12 by down-regulation of CXCR4, Biochem Biophys Res Commun 2013).

In this regard, CXCL12 promotes tumor growth, angiogenesis, and metastasis in hepatocellular carcinoma (HCC). It has been previously known that high CXCR4 expression may indicate a poor prognosis in HCC patients. Tumor-infiltrating myeloid-derived suppressor cells (MDSCs) also express CXCR4 and migrate toward CXCL12. Therefore, CXCL12 inhibition not only directly blocks HCC growth, but also alters the tumor environment (angiogenesis, MDSCs), etc., which can make HCC patients more sensitive to existing treatments. Therefore, indirect regulation of CXCL12 and CXCR4 by CXCR7 agonists has therapeutic potential for hepatitis, liver cancer, and even liver fibrosis (Stromal cell-derived factor-1(SDF-1) as a target in liver diseases TC-14012, Am J Physiol Gastrointest Liver Physiol 2016).

In one embodiment, the compound, a stereoisomer, isotopically labeled compound, hydrate, solvate, or pharmaceutically acceptable salt thereof according to the present invention may be useful for the treatment of cancer through selective modulatory activity against CXCR7.

As used herein, “cancer” is a general term for a disease caused by cells that have aggressive characteristics in which cells divide and grow while ignoring normal growth limits, invasive characteristics in which cells invade surrounding tissues, and metastatic characteristics in which cells spread to other parts of the body. For example, the cancer may include glioma, mesothelioma, melanoma, lymphoma, leukemia, adenocarcinoma, breast cancer, ovarian cancer, cervical cancer, leukemia, lymphoma, prostate cancer, and Burkitt's lymphoma, head and neck cancer, colon cancer, colorectal cancer, non-small cell lung cancer, small cell lung cancer, esophageal cancer, gastric cancer, pancreatic cancer, hepatobiliary cancer, gallbladder cancer, small intestine cancer, rectal cancer, kidney cancer, bladder cancer, prostate cancer, genital cancer, urethral cancer, testicular cancer, cervical cancer, vaginal cancer, uterine cancer, ovarian cancer, thyroid cancer, parathyroid cancer, adrenal cancer, pancreatic endocrine cancer, glioblastoma, bone cancer, skin cancer, retinoblastoma, Hodgkin's lymphoma or non-Hodgkin's lymphoma, but is not limited thereto. In one embodiment, the cancer for which the compound according to the present invention can exert a therapeutic effect through selective modulatory activity against CXCR7 is hepatobiliary cancer. In one embodiment, the cancer for which the compound according to the present invention can exert a therapeutic effect through selective modulatory activity against CXCR7 is breast cancer. In particular, the present inventors have confirmed that the compound according to the present invention exhibit an inhibitory effect on cancer metastasis by inhibiting CXCL12-mediated breast cancer cell migration.

CXCR7 agonists can inhibit tumor progression by binding to CXCL12 and blocking its interaction with CXCR4 in environments where the CXCL12/CXCR4 pathway is activated and cancer cell migration and metastasis are promoted. In addition, CXCR7 agonists can inhibit cancer metastasis and tumor spread by reducing the ability of cancer cells to migrate and invade surrounding tissues in the tumor microenvironment. CXCR7 agonists have also been reported to inhibit angiogenesis around tumors, thereby preventing the delivery of oxygen and nutrients to cancer cells. They also have been reported to modulate the recruitment of immune cells, thereby altering the tumor microenvironment. Specifically, CXCR7 acts as a competitive receptor for CXCL12 in breast cancer cells, thereby blocking CXCR4 signaling and inhibiting tumor metastasis (CXCL12 Retargeting of an Oncolytic Adenovirus Vector to the Chemokine CXCR4 and CXCR7 Receptors in Breast Cancer, Journal of Cancer Therapy, 12(6), 311-336; Opposing roles of CXCR4 and CXCR7 in breast cancer metastasis, Breast Cancer Research, 13(6):R128; Endothelial CXCR7 Regulates Breast Cancer Metastasis, Oncogene, 35, 1716-1724; Effects of the chemokine CXCL12 and combined internalization of its receptors CXCR4 and CXCR7 in human MCF-7 breast cancer cells, Cell Tissue Research, 357:253-266). In addition, it has been reported that treatment of breast cancer cell lines with VUF11207, a CXCR7 agonist, effectively reduces cancer cell migration and invasiveness by binding to both receptors, internalizing both CXCR4 and CXCR7 into the cells and inhibiting their cell surface expression (VUF11207, a Biased Ligand for CXCR4 and ACKR3, Induces Internalization and Degradation of Both Receptors in Breast Cancer Cell Lines; Int J Mol Sci. 2018 Nov. 14; 19(11):3592). Furthermore, CXCR7 has been reported to play an important role in regulating the recruitment of immune cells, suppressing immune evasion of cancer cells, and promoting anticancer immune responses (CXCR4 and CXCR7 Signaling Pathways: A Focus on the Cross-Talk Between Cancer Cells and Tumor Microenvironment, Frontiers in Oncology, 11:591386). CXCR7 activation in nervous system carcinoma such as glioblastoma has recently been reported to suppress tumor progression by inhibiting tumor motility and invasiveness and enhancing neural tissue protection mechanisms (CXCL12 modulation of CXCR4 and CXCR7 activity in human glioblastoma stem-like cells and regulation of the tumor microenvironment, Frontiers in Cellular Neuroscience, 8:144; CXCR7 activation evokes the anti-PD-L1 antibody against glioblastoma by remodeling CXCL12-mediated immunity; Liu et al, Cell Death & Disease, volume 15, 434, 2024). In a mouse model of glioblastoma, knockdown of CXCR7 resulted in worse survival outcomes and increased PD-L1 expression compared to the control group. In an ex vivo T cell experiment, VUF11207, a selective CXCR7 agonist, enhanced cytotoxicity against tumor cells and reversed immunosuppression in a glioblastoma cell-macrophage-T cell coculture. VUF11207, a CXCR7 agonist, suppresses PD-L1 expression and restores the function of intratumoral CD8+ T cells, resulting in a synergistic effect that improves survival rate when used in combination with anti-PD-L1 antibodies. Therefore, it is proposed as a novel therapeutic approach to enhance the efficacy of immune checkpoint inhibitors in glioblastoma (CXCR7 activation evokes the anti-PD-L1 antibody against glioblastoma by remodeling CXCL12-mediated immunity; Liu et al, Cell Death & Disease volume 15, 434, 2024). Targeting CXCR7 as a therapeutic strategy presents a promising approach for suppressing cancer progression and overcoming the limitations of existing anticancer treatments through these diverse mechanisms.

In one embodiment, the compound, a stereoisomer, isotopically labeled compound, hydrate, solvate, or pharmaceutically acceptable salt thereof according to the present invention may be useful for the treatment of autoimmune disorder through selective modulatory activity against CXCR7. For example, the autoimmune disorder may include multiple sclerosis, rheumatoid arthritis, inflammatory bowel disease, systemic lupus erythematosus, lupus nephritis, interstitial cystitis, celiac disease, autoimmune encephalomyelitis, dehydration disease, osteoarthritis, or type I diabetes, but is not limited thereto. For example, an autoimmune disorder that can be treated with the compound of the present invention, etc. may be multiple sclerosis. The present inventors have confirmed that the compound of the present invention exhibits excellent therapeutic effects on autoimmune diseases in an autoimmune disease model, particularly in the EAE (Experimental Autoimmune Encephalomyelitis) mouse model, a widely known multiple sclerosis model.

In one embodiment, the compound, a stereoisomer, isotopically labeled compound, hydrate, solvate, or pharmaceutically acceptable salt thereof according to the present invention may be useful for the treatment of osteoporosis through selective modulatory activity against CXCR7.

It has been reported that CXCR7 agonists can attenuate the effects of LPS-induced RANKL and TNF-α-related cytokines, while simultaneously reducing osteoclast formation and bone resorption induced by these cytokines (CXC receptor 7 agonist acts as a C-X-C motif chemokine ligand 12 inhibitor to ameliorate osteoclastogenesis and bone resorption, Molecular Medicine REPORTS 2022, 25(3), 78). Without being bound by theory, the compound of the present invention having selective modulatory activity against CXCR7 may be useful for the treatment of osteoporosis, which is caused by an imbalance between bone formation and bone resorption.

In one embodiment, the compound, a stereoisomer, isotopically labeled compound, hydrate, solvate, or pharmaceutically acceptable salt thereof according to the present invention may be useful for the treatment of pulmonary hypertension through selective modulatory activity against CXCR7. The pulmonary hypertension may include pulmonary arterial hypertension or chronic thromboembolic pulmonary hypertension, but is not limited thereto.

CXCR7 agonism/activation is a promising mechanism for the treatment of pulmonary arterial hypertension (PAH), and can inhibit disease progression through various mechanisms. CXCR7 activation is known to alleviate pulmonary endothelial cell damage caused by PAH by promoting the survival and function of vascular endothelial cells and inhibiting apoptosis (A role for the CXCL12 receptor, CXCR7, in the pathogenesis of human pulmonary vascular disease, European Respiratory Journal, 39, 1415-1424). It suppresses inflammation in the lungs through anti-inflammatory effects that reduce the inflammatory response that promotes PAH progression by inhibiting the activation of inflammatory cells, and regulates the CXCL12 signaling pathway together with CXCR4 to maintain a balance in angiogenesis and regeneration processes, and reduces pulmonary blood pressure increase by inhibiting CXCR4-dependent pathways (The Pivotal Role of CXCR7 in Stabilization of the Pulmonary Epithelial Barrier in Acute Pulmonary Inflammation, The Journal of Immunology, 198(6):2403-2413; A role for the CXCL12 receptor, CXCR7, in the pathogenesis of human pulmonary vascular disease, European Respiratory Journal, 39, 1415-1424). CXCR7 agonism is known to play an important role in alleviating vascular occlusion, one of the major pathological findings of PAH, by inhibiting smooth muscle cell proliferation during the vascular remodeling process (Rajagopal, S., Kim, J., Ahn, S., Craig, S., Lam, C. M., Gerard, N. P., . . . & Lefkowitz, R. J. (2010). “Beta-arrestin-but not G protein-mediated signaling by the ‘decoy’ receptor CXCR7.” Proceedings of the National Academy of Sciences, 107(2), 628-632). Based on this, a novel therapeutic approach can be proposed that aims to control the pathological progression of PAH through CXCR7 activation and ultimately improve the clinical condition of the disease.

In one embodiment, the compound, a stereoisomer, isotopically labeled compound, hydrate, solvate, or pharmaceutically acceptable salt thereof according to the present invention may be useful for the treatment of acute ischemic injury and thrombosis through selective modulatory activity against CXCR7.

Single-cell RNA sequencing analysis has shown that CXCR7 is the most abundantly expressed gene among 510 7TMR genes in cardiomyocytes and fibroblasts, and cardiomyocyte-specific CXCR7 null mice have been reported to exhibit significant cardiac enlargement and functional decline compared to control mice 4 weeks after myocardial infarction. These results suggest that CXCR7, abundantly expressed in cardiomyocytes, may function as a 0-arrestin-biased receptor, potentially providing cardiac protection, and that CXCR7 agonists have therapeutic effects in myocardial infarction (CXCR7 ameliorates myocardial infraction as a 0-arrestin-biased receptor, Scientific reports 2021).

Inhibition of platelet activation plays a pivotal role in the treatment of acute organ ischemia. CXCR7 expression on platelets in patients with coronary artery disease (CAD) is associated with clinical prognosis, and genetic deficiency of platelet CXCR7 is known to exacerbate tissue inflammation and systemic thromboinflammation by inducing platelet activation and damage to ischemic myocardium and brain tissue. In this regard, activation of platelet-CXCR7 via a CXCR7 agonist (VUF11207) has been reported to have inhibitory effects on platelet activation and thrombus formation, and to alleviate ischemic myocardium and brain tissue damage (ACKR3 regulates platelet activation and ischemia-reperfusion tissue injury, Nature Communications, 2022; Discovery and Development of First-in-Class ACKR3/CXCR7 Superagonists for Platelet Degranulation Modulation, J Med Chem, 2022). Without being bound by theory, the compound of the present invention having selective modulatory activity against CXCR7 may be useful for the treatment of acute organ ischemic injury, such as ischemic cardiac injury, and thrombosis.

As used herein, the term “treating” or “treatment” refers to inhibiting a disease, for example, inhibiting a disease, condition, or disorder in a subject who experiences or exhibits the pathology or signs of the disease, condition, or disorder, i.e., preventing further development of the pathology and/or signs, or ameliorating the disease, for example, ameliorating the disease, condition, or disorder in a subject who experiences or exhibits the pathology or signs of the disease, condition, or disorder, i.e., reversing the pathology and/or signs, for example, reducing the severity of the disease.

The pharmaceutical composition may comprise, in addition to the compound, a stereoisomer, isotopically labeled compound, hydrate, solvate, or pharmaceutically acceptable salt thereof of the present invention, an additional therapeutically active agent. In this case, the compound of the present invention and the additional therapeutically active agent may be in a single composition or separate compositions. For example, the compound of the present invention may be provided as a composition in an oral dosage form and the additional therapeutically active agent may be provided in a parenteral dosage form, or the compound of the present invention may be provided as a parenteral dosage form and the additional therapeutically active agent may be provided in an oral dosage form. When the compound of the present invention is used for the treatment of fibrosis, it may be administered simultaneously, sequentially, or separately with, for example, nintedanib, pirfenidone, etc. When the compound of the present invention is used for the treatment of pulmonary arterial hypertension, it may be administered simultaneously, sequentially, or separately with, for example, sildenafil, etc. When the compound of the present invention is used for the treatment of multiple sclerosis, it may be administered simultaneously, sequentially, or separately with, for example, fingolimod (FTY720), etc.

The pharmaceutical composition may comprise a pharmaceutically acceptable carrier. The term “carrier” is used to mean an excipient, diluent, or adjuvant. For example, the carrier may be selected from the group consisting of lactose, dextrose, sucrose, sorbitol, mannitol, xylitol, erythritol, maltitol, starch, acacia gum, alginate, gelatin, calcium phosphate, calcium silicate, cellulose, methyl cellulose, polyvinyl pyrrolidone, water, physiological saline, a buffer such as PBS, methyl hydroxybenzoate, propyl hydroxybenzoate, talc, magnesium stearate, and mineral oil. The composition may comprise a filler, an anticoagulant, a lubricant, a wetting agent, a flavoring agent, an emulsifier, a preservative, or a combination thereof.

The pharmaceutical composition may be prepared in any dosage form using conventional methods. The composition may be formulated, for example, as an oral dosage form (e.g., a powder, tablet, capsule, syrup, pill, or granule) or a parenteral dosage form (e.g., an injection). In addition, the composition may be formulated as a systemic dosage form or a topical dosage form.

In the pharmaceutical composition, the solid dosage form for oral administration may be a tablet, pill, powder, granule, or capsule. The solid dosage form may further comprise an excipient. Examples of such excipients may include starch, calcium carbonate, sucrose, lactose, or gelatin. In addition, the solid dosage form may further comprise a lubricant such as magnesium stearate or talc. In the pharmaceutical composition, the liquid dosage form for oral administration may be a suspension, an oral solution, an emulsion, or a syrup. The liquid dosage form may comprise water or liquid paraffin. The liquid dosage form may comprise an excipient, such as a wetting agent, a sweetener, a flavoring agent, or a preservative. In the pharmaceutical composition, a dosage form for parenteral administration may be a sterilized aqueous solution, a non-aqueous solution, a suspension, an emulsion, a lyophilized formulation, or a suppository. The non-aqueous solution or suspension may comprise a vegetable oil or an ester. The vegetable oil may be, for example, propylene glycol, polyethylene glycol, or olive oil. The ester may be, for example, ethyl oleate. The suppository base may be witepsol, macrogol, Tween 61, cacao butter, laurin butter, or glycerogelatin.

The pharmaceutical composition comprises a compound, a stereoisomer, isotopically labeled compound, solvate, or pharmaceutically acceptable salt thereof according to one aspect as an active ingredient of the pharmaceutical composition. The term “active ingredient” refers to a physiologically active substance used to achieve pharmacological activity (e.g., an antifibrotic effect).

The pharmaceutical composition may comprise an effective amount of a compound, a stereoisomer, isotopically labeled compound, solvate, or pharmaceutically acceptable salt thereof according to one aspect. The term “effective amount” refers to an amount sufficient to exhibit the effect of preventing or treating a disease when administered to a subject in need of prevention or treatment. The effective amount can be appropriately selected by those of ordinary skill in the art depending on the cell or subject being treated. The preferred dosage of the pharmaceutical composition varies depending on the subject's condition and body weight, the severity of the disease, the drug form, the route of administration, and the duration of administration, but can be appropriately selected by those of ordinary skill in the art. However, the compound, a stereoisomer, isotopically labeled compound, solvate, or pharmaceutically acceptable salt thereof may be administered in divided doses, for example, from about 0.0001 mg/kg to about 100 mg/kg, or from about 0.001 mg/kg to about 100 mg/kg, once to 24 times daily, once to 7 times every 2 days to 1 week, or once to 24 times every 1 to 12 months. In the pharmaceutical composition, the compound, a stereoisomer, isotopically labeled compound, solvate, or pharmaceutically acceptable salt thereof may be included in an amount of from about 0.0001 wt % to about 10 wt %, or from about 0.001 wt % to about 1 wt %, based on the total weight of the entire composition.

The administration method may be oral or parenteral. Examples of administration routes may include oral, transdermal, subcutaneous, rectal, intravenous, intraarterial, intraperitoneal, intramuscular, intrasternal, topical, intranasal, intratracheal, or intradermal routes. The composition may be administered systemically or locally, and may be administered alone or in combination with other therapeutically active agents.

In another aspect, there is provided a method for treating CXCR7-mediated diseases, comprising administering a compound, a stereoisomer, isotopically labeled compound, hydrate, solvate, or pharmaceutically acceptable salt thereof according to one aspect to a subject. The compound, the stereoisomer, the isotopically labeled compound, the hydrate, the solvate, the pharmaceutically acceptable salt, and the treatable disease are the same as described above.

In another aspect, there is provided a use of a compound, a stereoisomer, isotopically labeled compound, hydrate, solvate, or pharmaceutically acceptable salt thereof according to one aspect, for use in the manufacture of a medicament for treating CXCR7-mediated diseases. The compound, the stereoisomer, the isotopically labeled compound, the hydrate, the solvate, the pharmaceutically acceptable salt, and the treatable disease are the same as described above.

### Effects of Invention

According to the compound according to one aspect, a use thereof as a CXCR7 receptor modulator, a pharmaceutical composition comprising the same, and a use thereof for the treatment of CXCR7-mediated diseases, the compound has excellent CXCR7 modulating activity and may be useful for the treatment of fibrosis, cholestatic liver disease, inflammatory disease, angiogenic disease, cancer, autoimmune disorder, osteoporosis, pulmonary hypertension, acute ischemic injury, and thrombosis.

### BRIEF DESCRIPTION OF DRAWINGS

FIG. 1 is a diagram showing the results of BALF absolute differential cell count in a bleomycin-induced IPF model. Data are presented as mean±standard deviation; #p<0.05, ##p<0.01, ###p<0.001 (compared to normal control group); *p<0.05, **p<0.01, ***p<0.001 (compared to bleomycin+vehicle); one-way ANOVA or two-way ANOVA followed by Dunnett's multiple comparisons test; n=10-12.

FIG. 2 is a diagram showing the results of measuring lung hydroxyproline content in a bleomycin-induced IPF model. Data are presented as mean±standard deviation; ###p<0.001 (compared to normal control group); ***p<0.001 (compared to bleomycin+vehicle); one-way ANOVA followed by Dunnett's multiple comparisons test; n=10-12.

FIG. 3 is a diagram showing the results of evaluating the Ashcroft fibrosis score in a bleomycin-induced IPF model. Data are presented as mean±standard deviation; ###p<0.001 (compared to normal control group); **p<0.01, ***p<0.001 (compared to bleomycin+vehicle); one-way ANOVA followed by Dunnett's multiple comparisons test; n=10-12.

FIG. 4 is a diagram showing the results of measuring hepatic hydroxyproline in a carbon tetrachloride-induced liver fibrosis model. Data are presented as mean±standard deviation; ###p<0.001 (compared to normal control group); ***p<0.001 (compared to CCl₄+vehicle); one-way ANOVA followed by Dunnett's multiple comparisons test; n=10.

FIG. 5 is a diagram showing the results of measuring Ishak fibrosis score in a carbon tetrachloride-induced liver fibrosis model. Data are presented as mean±standard deviation; ###p<0.001 (compared to normal control group); **p<0.01, ***p<0.001 (compared to CCl₄+vehicle); one-way ANOVA followed by Dunnett's multiple comparisons test; n=10.

FIG. 6 is a diagram showing the results of measuring ALT in a carbon tetrachloride-induced liver fibrosis model. Data are presented as mean±standard deviation; ###p<0.001 (compared to normal control group); *p<0.05 (compared to CCl₄+vehicle); one-way ANOVA followed by Dunnett's multiple comparisons test; n=10.

FIG. 7 is a diagram showing the results of clinical scoring in a model of autoimmune neuroinflammation induced by MOG 35-55.

FIG. 8 is a diagram showing the results of measuring right ventricular systolic pressure (RVSP) in a model of pulmonary arterial hypertension induced by monocrotaline administration. Data are presented as mean±standard deviation; ****P<0.0001 (compared to normal control group); #P<0.05 (compared to pulmonary arterial hypertension model)

FIG. 9 is a diagram showing the results of measuring the ratio of right ventricular weight to left ventricular plus septal weight (RV/LV+S) in a model of pulmonary arterial hypertension induced by monocrotaline administration. Data are presented as mean±standard deviation; ****P<0.0001 (compared to normal control group); #P<0.05 (compared to pulmonary arterial hypertension model)

FIG. 10 is a diagram showing the results of measuring right ventricular wall thickness in a model of pulmonary arterial hypertension induced by monocrotaline administration. Data are presented as mean±standard deviation; ****P<0.0001 (compared to normal control group); #P<0.05, ###P<0.001 (compared to pulmonary arterial hypertension model)

FIG. 11 is a diagram showing the results of measuring the inhibitory effect of CXCL12 on cell migration of MDA-MB-231 breast cancer cells induced by CXCL12. **P<0.01 (compared to CXCL12 treated group)

FIG. 12 is a diagram showing the results of measuring AST, ALT, ALP, and total bilirubin (TBIL) in a cholestatic liver disease model induced by bile duct ligation. *p<0.05, **p<0.01, ***p<0.001 (compared to the bile duct ligation model)

FIG. 13 is a diagram showing the results of the HE Score and α-SMA immunohistochemistry (IHC) quantitative morphometry, as evaluated by the criteria in Table 6 of the present application after H&E staining in a cholestatic liver disease model induced by bile duct ligation. *p<0.05, **p<0.01, ***p<0.001 (compared to the bile duct ligation model)

### MODE FOR CARRYING OUT THE INVENTION

The following examples are provided to illustrate the present disclosure and should not be construed as limiting the scope of the present disclosure in any way. All functionally equivalent methods are within the scope of the present disclosure. Various modifications of the present disclosure, in addition to those described herein, will become apparent to those of ordinary skill in the art from the foregoing description. Such modifications fall within the scope of the appended claims.

The meanings of the abbreviations used in the following examples are as follows. Abbreviations not listed below have the meanings commonly used in the relevant fields.
THF: tetrahydrofuran
TEA: triethanolamine
DMF: N,N-dimethylformamide
DCM: dichloromethane
TFA: trifluoroacetic acid
NaOAc: sodium acetate
EA: ethyl acetate
TPP: triphenylphosphine
TMEDA: N,N,N′,N′-tetramethylethylenediamine

### Preparation Example S1: 4-chloro-1H-pyrazolo[3,4-d]pyrimidin-6-amine (Intermediate S1)

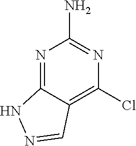 [SMILES: Nc1nc(Cl)c2cn[nH]c2n1]

To a stirred solution of 2-amino-4,6-dichloropyrimidine-5-carbaldehyde (100 g, 521 mmol) in a mixture of THF (700 mL) and water (300 mL) were added TEA (87.7 mL, 625 mmol) and hydrazine monohydrate (30.7 mL, 625 mmol) at 0° C., and the resulting mixture was stirred at 50° C. for 3 hours. After the reaction was completed, the solvent was distilled off, and the resulting residue was filtered, washed with diethyl ether, and dried in vacuo to afford Intermediate S1 (80 g, 90.58% yield) as a pale yellow solid. LCMS: m/z=170 (M+1, ESI+).

### Preparation Example S2: 5-(6-amino-1H-pyrazolo[3,4-d]pyrimidin-4-yl)isophthalonitrile (Intermediate S2)

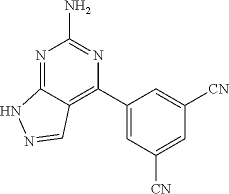 [SMILES: N#Cc1cc(C#N)cc(-c2nc(N)nc3[nH]ncc23)c1]

To a stirred solution of Intermediate S1 (5 g, 29.5 mmol) in a mixture of THF (144 mL) and H₂O (36 mL) in a sealed tube were added 5-(4,4,5,5-tetramethyl-1,3,2-dioxaborolan-2-yl)isophthalonitrile (8.99 g, 35.4 mmol) and K₂CO₃ (8.15 g, 59 mmol) at 25° C. and degassed with argon for 10 minutes. Thereafter, Pd(PPh₃)₄ (1.7 g, 1.47 mmol) was added, and the resulting mixture was stirred at 110° C. for 12 hours. After the reaction was completed, the reaction mixture was cooled to room temperature, filtered through a Celite bed, and washed with ethyl acetate (250 mL×2) and 10% MeOH in DCM (500 ml×6). The filtrate was concentrated under reduced pressure. The residue was triturated with 10% acetonitrile in diethyl ether to afford Intermediate S2 (2.4 g, 86% yield) as a pale yellow solid. LCMS: m/z=262 (M+1, ESI+).

### Preparation Example S3: 4-chloro-1-(3-(trifluoromethyl)benzyl)-1H-pyrazolo[3,4-d]pyrimidin-6-amine (Intermediate S3)

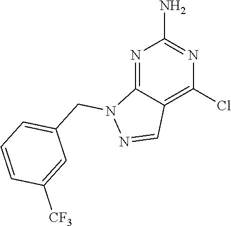 [SMILES: Nc1nc(Cl)c2cnn(Cc3cccc(C(F)(F)F)c3)c2n1]

To a stirred solution of Intermediate S1 (5 g, 29.5 mmol) in dry DMF was added K₂CO₃ (8.15 g, 59 mmol) at 0° C. After 10 minutes, 1-(bromomethyl)-3-(trifluoromethyl)benzene (4.52 mL, 29.5 mmol) was added, and the resulting mixture was stirred at 25° C. for 4 hours. After the reaction was completed, the reaction mixture was quenched with ice water (150 mL), and the resulting precipitate was filtered, washed with ice water (100 mL×2), and dried in vacuo. The residue was purified by column chromatography to afford Intermediate S3 (1.7 g, 17.59% yield) as an off-white solid. LCMS: m/z=328 (M+1, ESI+).

### Preparation Example S4: 5-(2-amino-7H-pyrrolo[2,3-d]pyrimidin-4-yl)isophthalonitrile (Intermediate S4)

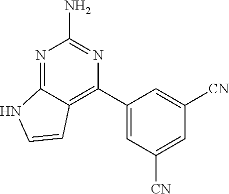 [SMILES: N#Cc1cc(C#N)cc(-c2nc(N)nc3[nH]ccc23)c1]

To a stirred solution of 4-chloro-7H-pyrrolo[2,3-d]pyrimidin-2-amine (4 g, 23.7 mmol) and 5-(4,4,5,5-tetramethyl-1,3,2-dioxaborolan-2-yl)isophthalonitrile (7.23 g, 28.5 mmol) in a mixture of THF (80 mL) and water (20 mL) was added K₂CO₃ (6.56 g, 47.5 mmol) at 25° C., and the mixture was degassed with argon for 10 minutes. Thereafter, Pd(PPh₃)₄ (1.37 g, 1.19 mmol) was added, and the resulting mixture was stirred at 100° C. for 16 hours. After the reaction was completed, the reaction mixture was filtered through a Celite bed, and the bed was washed with EA (600 mL) and MeOH (200 mL). The filtrate was collected and concentrated to obtain a residue. The residue was triturated with 5% MeOH in DCM to afford Intermediate S4 (2.5 g, 62% yield) as a pale yellow solid. LCMS: m/z=261 (M+1, ESI+).

### Preparation Example S5: 5-(2-amino-9H-purin-6-yl)isophthalonitrile (Intermediate S5)

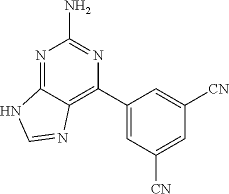 [SMILES: N#Cc1cc(C#N)cc(-c2nc(N)nc3[nH]cnc23)c1]

To a stirred solution of 6-chloro-9H-purin-2-amine (6 g, 22.98 mmol) and 5-(4,4,5,5-tetramethyl-1,3,2-dioxaborolan-2-yl)isophthalonitrile (7.59 g, 29.88 mmol) in a mixture of 1,4-dioxane (80 mL) and water (20 mL) was added K₂CO₃ (9.51 g, 68.96 mmol) at 25° C., and the mixture was degassed with argon for 10 minutes. Thereafter, Pd(dppf)Cl₂·DCM (1.87 g, 2.2 mmol) was added, and the resulting mixture was stirred at 100° C. for 16 hours. After the reaction was completed, the reaction mixture was filtered through a Celite bed, and the bed was washed with EA (600 mL) and MeOH (200 mL). The filtrate was collected and concentrated. The residue was purified by column chromatography to afford Intermediate S5 (3 g, crude) as a pale yellow solid. LCMS: m/z=262 (M+1, ESI+).

### Preparation Example S6: 5-(5-amino-3H-[1,2,3]triazolo[4,5-d]pyrimidin-7-yl)isophthalonitrile trifluoroacetate (Intermediate S6)

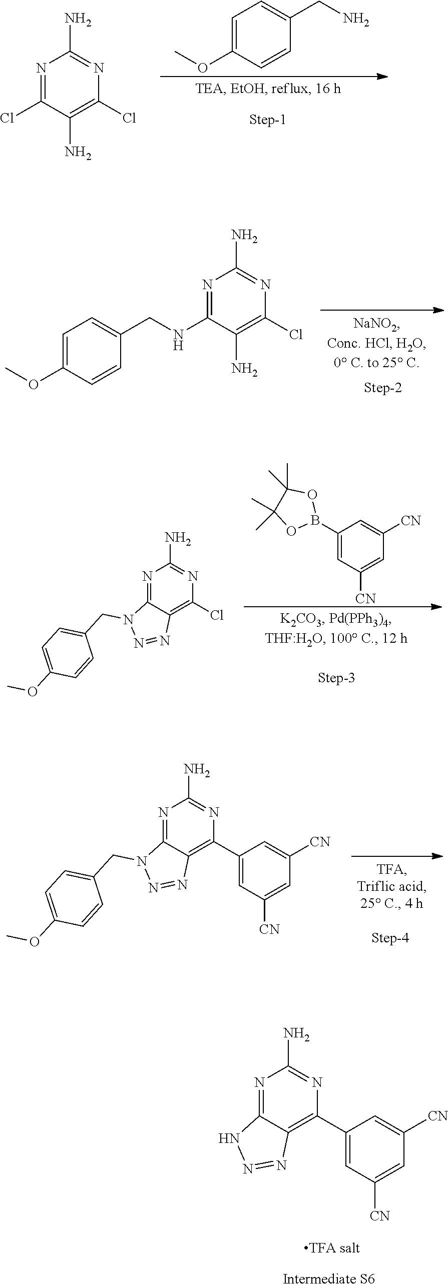

### Step 1: 6-chloro-N4-(4-methoxybenzyl)pyrimidine-2,4,5-triamine

To a stirred solution of 4,6-dichloropyrimidine-2,5-diamine (5 g, 27.9 mmol) in ethanol (100 mL) were added triethylamine (9.75 mL, 69.8 mmol) and (4-methoxyphenyl)methanamine (7.3 mL, 55.9 mmol) at 0° C., and the resulting mixture was heated to reflux at 80° C. for 16 hours. After the reaction was completed, the reaction mixture was cooled to 25° C., and the solvent was evaporated. The crude product was dissolved in DCM (100 mL), washed with water (25 mL) and brine (20 mL), dried over anhydrous Na₂SO₄, and concentrated. The residue was purified by column chromatography to afford 6-chloro-N4-(4-methoxybenzyl)pyrimidine-2,4,5-triamine (6.0 g, 76.82% yield) as an orange solid. LCMS: m/z=280 (M+1, ESI+).

### Step 2: 7-chloro-3-(4-methoxybenzyl)-3H-[1,2,3]triazolo[4,5-d]pyrimidin-5-amine

To a stirred solution of 6-chloro-N4-(4-methoxybenzyl)pyrimidine-2,4,5-triamine (2.5 g, 8.94 mmol) in conc. HCl (30 mL) was added dropwise a solution of NaNO₂ (925 mg, 13.4 mmol) in water (3 mL) at 0° C. for 20 minutes. Thereafter, the resulting mixture was stirred at 0° C. for 30 minutes. After the reaction was completed, the reaction mixture was poured into a saturated NaHCO₃ solution with vigorous stirring. The resulting precipitate was filtered and dried in vacuo to afford 7-chloro-3-(4-methoxybenzyl)-3H-[1,2,3]triazolo[4,5-d]pyrimidin-5-amine (1.8 g, 69.49%) as an off-white solid. LCMS: m/z=291 (M+1, ESI+).

### Step 3: 5-(5-amino-3-(4-methoxybenzyl)-3H-[1,2,3]triazolo[4,5-d]pyrimidin-7-yl)isophthalonitrile

To a stirred solution of 7-chloro-3-(4-methoxybenzyl)-3H-[1,2,3]triazolo[4,5-d]pyrimidin-5-amine (2 g, 6.89 mmol) in 1,4-dioxane (25 mL) and water (8 mL) were added 5-(4,4,5,5-tetramethyl-1,3,2-dioxaborolan-2-yl)isophthalonitrile (2.1 g, 8.27 mmol) and K₂CO₃ (1.90 g, 13.79 mmol) at 25° C., and the mixture was degassed with argon for 10 minutes. Thereafter, Pd(dppf)Cl₂·DCM (0.281 g, 0.344 mmol) was added, and the resulting mixture was stirred at 100° C. for 12 hours. After the reaction was completed, the reaction mixture was diluted with EA (100 mL), washed with water (30 mL) and brine (30 mL), and concentrated. The residue was purified by column chromatography to afford 5-(5-amino-3-(4-methoxybenzyl)-3H-[1,2,3]triazolo[4,5-d]pyrimidin-7-yl)isophthalonitrile (1.5 g, 57.03% yield) as an orange solid. LCMS: m/z=383 (M+1, ESI+).

### Step 4: 5-(5-amino-3H-[1,2,3]triazolo[4,5-d]pyrimidin-7-yl)isophthalonitrile trifluoroacetate (Intermediate S6)

To a stirred solution of 5-(5-amino-3-(4-methoxybenzyl)-3H-[1,2,3]triazolo[4,5-d]pyrimidin-7-yl)isophthalonitrile (1.3 g, 3.40 mmol) in TFA (20 mL) was added trifluoromethanesulfonic acid (3.46 mL, 39.2 mmol) at 0° C., and the resulting mixture was stirred at 25° C. for 4 hours. After the reaction was completed, the volatiles were evaporated, and the residue was triturated with diethyl ether to afford Intermediate S6 (1.2 g TFA salt, quantitative yield) as an orange solid. LCMS: m/z=263 (M+1, ESI+).

### Preparation Example S7: 5-(2-amino-5-methyl-7H-pyrrolo[2,3-d]pyrimidin-4-yl)isophthalonitrile (Intermediate S7)

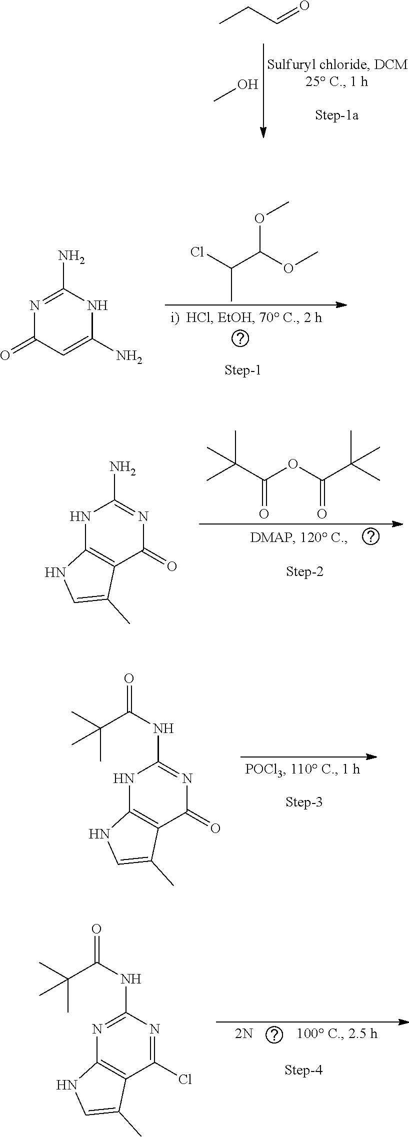

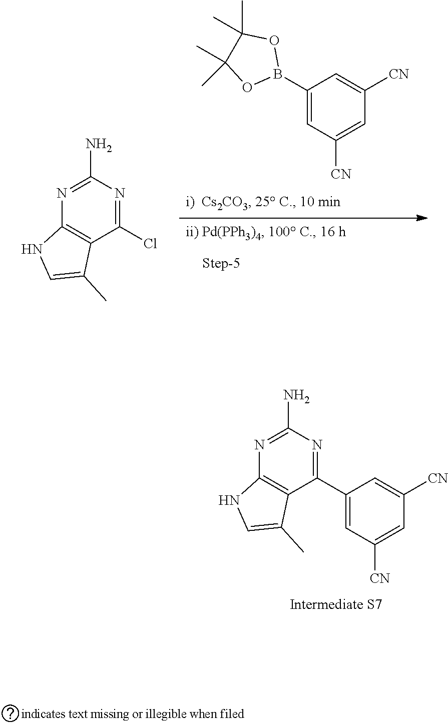 [SMILES: Cc1c[nH]c2nc(N)nc(Cl)c12 ; CC1(C)OB(c2cc(C#N)cc(C#N)c2)OC1(C)C ; Cc1c[nH]c2nc(N)nc(-c3cc(C#N)cc(C#N)c3)c12]

### Step 1a: 2-chloro-1,1-dimethoxypropane

To a stirred solution of sulfuryl chloride in DCM (1 M, 100 mL) was added propionaldehyde (70 mL, 976 mmol) at 0° C. over 10 minutes. After stirring at 25° C. for 1 hour, methanol (240 mL) was added to the reaction mixture over 15 minutes. Vigorous gas evolution was observed during the addition. After stirring at 25° C. for 2.5 hours, the DCM was distilled off. To the reaction mixture was added saturated NaHCO₃ solution (15 mL), extracted with ether, dried over Na₂SO₄, and concentrated under reduced pressure to afford 2-chloro-1,1-dimethoxypropane (40 g, crude, quantitative) as a colorless semi-solid. Based on TLC, the residue was used as it is in the next step.

### Step 1: 2-amino-5-methyl-1,7-dihydro-4H-pyrrolo[2,3-d]pyrimidin-4-one

A mixture of 2-chloro-1,1-dimethoxypropane (7.5 mL, 39.6 mmol) in 1N HCl (22 mL) and ethanol (7 mL) was stirred at 70° C. for 2 hours. The mixture was cooled to 25° C. A mixture of 2,6-diaminopyrimidin-4(1H)-one (5 g, 39.6 mmol) and NaHCO₃ in water (50 mL) was stirred at 50° C. for 15 minutes. Thereafter, the hydrolyzed acetal mixture was slowly added and stirred for 15 minutes. The mixture was cooled to 0° C., and the reaction mixture was quenched with saturated NH₄Cl solution. The residue was treated with ice, and the resulting precipitate was filtered to afford 2-amino-5-methyl-1,7-dihydro-4H-pyrrolo[2,3-d]pyrimidin-4-one (5 g, crude, quantitative). LCMS: m/z=165 (M+1, ESI+).

### Step 2: N-(5-methyl-4-oxo-4,7-dihydro-1H-pyrrolo[2,3-d]pyrimidin-2-yl)pivalamide

A stirred mixture of 2-amino-5-methyl-1,7-dihydro-4H-pyrrolo[2,3-d]pyrimidin-4-one (15 g, 91.46 mmol) and N,N-dimethyl-4-pyridylamine (558 mg, 4.57 mmol) in pivalic anhydride (82 mL) was stirred at 120° C. for 1.5 hours. The reaction mixture was cooled to 0° C., cold ether was added to the reaction mixture, and the resulting precipitate was filtered to afford N-(5-methyl-4-oxo-4,7-dihydro-1H-pyrrolo[2,3-d]pyrimidin-2-yl)pivalamide (14 g, crude, quantitative) as a pale yellow solid. LCMS: m/z=249 (M+1, ESI+).

### Step 3: N-(5-methyl-4-oxo-4,7-dihydro-1H-pyrrolo[2,3-d]pyrimidin-2-yl)pivalamide

A mixture of N-(5-methyl-4-oxo-4,7-dihydro-1H-pyrrolo[2,3-d]pyrimidin-2-yl)pivalamide (14 g, 56.4 mmol) in POCl₃ (113 mL, 141 mmol) was heated at 110° C. for 1 hour. After the reaction was completed, excess of POCl₃ was removed from the reaction mixture in vacuo, and the residue was added to a solution of ice and 25% ammonia. The mixture was stirred vigorously for 30 minutes, and the resulting precipitate was filtered to afford N-(5-methyl-4-oxo-4,7-dihydro-1H-pyrrolo[2,3-d]pyrimidin-2-yl)pivalamide (15 g, crude, quantitative). LCMS: m/z=267 (M+1, ESI+).

### Step 4: 4-chloro-5-methyl-7H-pyrrolo[2,3-d]pyrimidin-2-amine

A mixture of N-(5-methyl-4-oxo-4,7-dihydro-1H-pyrrolo[2,3-d]pyrimidin-2-yl)pivalamide (15 g, 56.2 mmol) in 2N NaOH (200 mL) was stirred at 120° C. for 2.5 hours. The reaction mixture was cooled to room temperature, and the resulting precipitate was filtered to afford 4-chloro-5-methyl-7H-pyrrolo[2,3-d]pyrimidin-2-amine (7 g, quantitative) as a pale yellow solid. LCMS: m/z=183 (M+1, ESI+).

### Step 5: 5-(2-amino-5-methyl-7H-pyrrolo[2,3-d]pyrimidin-4-yl)isophthalonitrile (Intermediate S7)

To a stirred solution of 4-chloro-5-methyl-7H-pyrrolo[2,3-d]pyrimidin-2-amine (1.2 g, 6.59 mmol) in 1,4-dioxane (8 mL) and water (2 mL) were added 5-(4,4,5,5-tetramethyl-1,3,2-dioxaborolan-2-yl)isophthalonitrile (2.01 g, 7.91 mmol) and Cs₂CO₃ (6.44 g, 19.78 mmol) at 25° C., and the mixture was degassed with argon for 10 minutes. Thereafter, Pd(PPh₃)₄ (761 mg, 0.659 mmol) was added, and the mixture was stirred at 100° C. for 16 hours. After the reaction was completed, the reaction mixture was filtered through a Celite bed, and the bed was washed with EA (100 mL×5) and MeOH (100 mL). The filtrate was collected and concentrated. The residue was triturated with 5% MeOH in DCM to afford Intermediate S7 (1.2 g, crude, 66.44% yield) as a pale yellow solid. The crude product was used as it is as an intermediate. LCMS: m/z=275 (M+1, ESI+).

### Preparation Example S8: 5-(2-amino-6-methyl-7H-pyrrolo[2,3-d]pyrimidin-4-yl)isophthalonitrile (Intermediate S8)

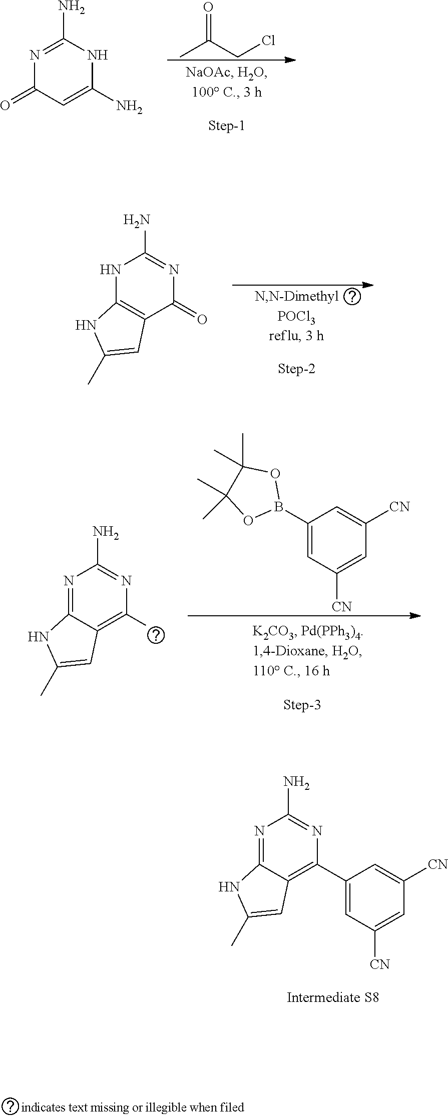

### Step 1: 2-amino-6-methyl-1,7-dihydro-4H-pyrrolo[2,3-d]pyrimidin-4-one

A suspension of 2,6-diamino-4(1H)-pyrimidinone (10 g, 79.3 mmol) and NaOAc (6.5 g, 79.3 mmol) in water (100 mL) was stirred at 100° C. After 10 minutes, 1-chloro-2-propanone (7.34 mL, 79.3 mmol) was slowly added, and the mixture was stirred at 100° C. for 3 hours. After the reaction was completed, the reaction mixture was filtered, washed with water (200 mL), and dried in vacuo to afford 2-amino-6-methyl-1,7-dihydro-4H-pyrrolo[2,3-d]pyrimidin-4-one (10.8 g, 83.01% yield) as an off-white solid. LCMS: m/z=165 (M+1, ESI+).

### Step 2: 4-chloro-6-methyl-7H-pyrrolo[2,3-d]pyrimidin-2-amine

A mixture of 2-amino-6-methyl-1,7-dihydro-4H-pyrrolo[2,3-d]pyrimidin-4-one (10.8 g, 65.8 mmol) in POCl₃ (110 mL, 660 mmol) and N,N-dimethylaniline (0.3 mL, 2.37 mmol) was stirred at 110° C. for 3 hours. After the reaction was completed, the reaction mixture was evaporated under reduced pressure. The residue was poured onto ice and basified with an aqueous solution of 25% ammonia (120 mL), and the precipitate was filtered and dried in vacuo to obtain 4-chloro-6-methyl-7H-pyrrolo[2,3-d]pyrimidin-2-amine (4.4 g, 36.63% yield) as an off-white solid. LCMS: m/z=183 (M+1, ESI+).

### Step 3: 5-(2-amino-6-methyl-7H-pyrrolo[2,3-d]pyrimidin-4-yl)isophthalonitrile (Intermediate S8)

To a stirred solution of 4-chloro-6-methyl-7H-pyrrolo[2,3-d]pyrimidin-2-amine (1.5 g, 8.24 mmol) and 5-(4,4,5,5-tetramethyl-1,3,2-dioxaborolan-2-yl)isophthalonitrile (2.51 g, 9.89 mmol) in a mixture of 1,4-dioxane (36 mL) and water (4 mL) was added K₂CO₃ (2.27 g, 16.48 mmol), and the mixture was degassed with argon for 10 minutes. Thereafter, Pd(dppf)Cl₂·DCM (0.33 g, 0.41 mmol) was added, and the mixture was stirred at 110° C. for 16 hours. After the reaction was completed, the reaction mixture was filtered through a Celite bed and washed with EA (600 mL). The organic layer was washed with water (100 mL), dried over Na₂SO₄, and concentrated. The residue was purified by column chromatography to afford Intermediate S8 (1.1 g, crude, 30% yield) as a yellow solid. LCMS: m/z=275 (M+1, ESI+).

### Preparation Example S9: 5-(2-amino-8-methyl-9H-purin-6-yl)isophthalonitrile (Intermediate S9)

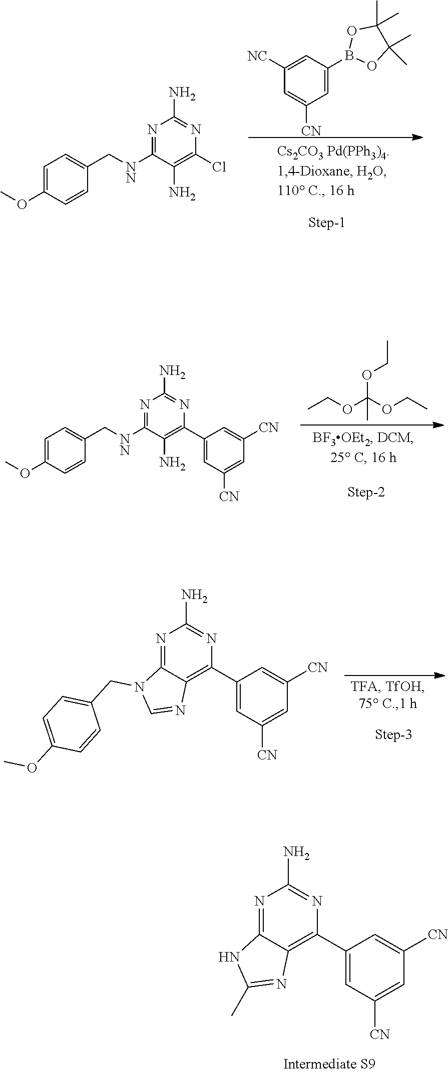

### Step 1: 5-(2,5-diamino-6-((4-methoxybenzyl)amino)pyrimidin-4-yl)isophthalonitrile

To a stirred solution of 6-chloro-N4-(4-methoxybenzyl)pyrimidine-2,4,5-triamine (4 g, 14.30 mmol) obtained in Step 1 of Preparation Example S6 and 5-(4,4,5,5-tetramethyl-1,3,2 -dioxaborolan-2-yl)isophthalonitrile (4.3 g, 17.16 mmol) in a mixture of 1,4-dioxane (36 mL) and water (4 mL) was added Cs₂CO₃ (13.9 g, 42.90 mmol) and degassed with argon for 10 minutes. Thereafter, Pd(PPh₃)₄ (1.65 g, 1.43 mmol) was added, and the mixture was stirred at 100° C. for 16 hours. After the reaction was completed, the reaction mixture was filtered through a Celite bed and washed with EA (600 mL). The organic phase was washed with water (100 mL), dried over Na₂SO₄, and concentrated. The residue was purified by column chromatography to afford 5-(2,5-diamino-6-((4-methoxybenzyl)amino)pyrimidin-4-yl)isophthalonitrile (4.8 g, crude, 67% yield) as a yellow solid. LCMS: m/z=372 (M+1, ESI+).

### Step 2: 5-(2-amino-9-(4-methoxybenzyl)-8-methyl-9H-purin-6-yl)isophthalonitrile

To a stirred solution of 5-(2,5-diamino-6-((4-methoxybenzyl)amino)pyrimidin-4-yl)isophthalonitrile (4.8 g, crude, 8.65 mmol) in dry DCM were added 1,1,1-triethoxyethane (2.1 g, 12.98 mmol) and BF₃·OEt₂ (1.2 g, 8.65 mmol) at 0° C., and the mixture was stirred at 25° C. for 16 hours. After the reaction was completed, the reaction mixture was basified (pH of about 7 to 8) with saturated NaHCO₃ solution and extracted with 10% MeOH in DCM (400 mL×3). The combined organic layer was washed with brine (100 mL), dried over Na₂SO₄, and concentrated. The residue was purified by column chromatography to afford 5-(2-amino-9-(4-methoxybenzyl)-8-methyl-9H-purin-6-yl)isophthalonitrile (2.28 g, 66.58% yield) as a yellow solid. LCMS: m/z=396 (M+1, ESI+).

### Step 3: 5-(2-amino-8-methyl-9H-purin-6-yl)isophthalonitrile (Intermediate S9)

To a stirred solution of 5-(2-amino-9-(4-methoxybenzyl)-8-methyl-9H-purin-6-yl)isophthalonitrile (1.8 g, 4.55 mmol) in TFA (20 ml) was added trifluoromethanesulfonic acid (6.8 g, 45.52 mmol), and the mixture was stirred at 75° C. for 1 hour. After the reaction was completed, the reaction mixture was quenched with ice and basified (pH of about 7 to 8) with saturated NaHCO₃ solution. The precipitated solid was collected by filtration to afford Intermediate S9 (1.2 g, 95.77% yield) as a yellow solid. LCMS: m/z=276 (M+1, ESI+).

### Preparation Example S10: 5-(6-amino-3-methyl-1H-pyrazolo[3,4-d]pyrimidin-4-yl)isophthalonitrile (Intermediate S10)

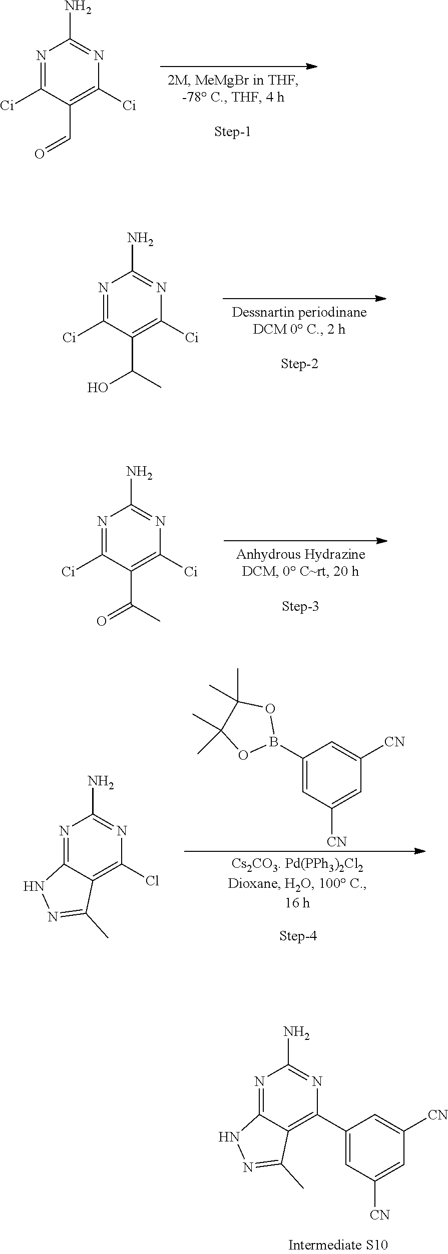

### Step 1: 1-(2-amino-4,6-dichloropyrimidin-5-yl)ethan-1-ol

To a solution of 2-amino-4,6-dichloropyrimidine-5-carbaldehyde (20 g, 104 mmol) in THF (50 mL) was added 2M MeMgBr (185 mL, 871 mmol) at −78° C., and the mixture was stirred at −78° C. for 4 hours. After the reaction was completed, the reaction mixture was quenched with saturated NH₄Cl solution and extracted with EA (350 mL×2). The combined organic layer was washed with brine (100 mL), dried over anhydrous Na₂SO₄, and concentrated. The residue was purified by column chromatography to afford 1-(2-amino-4,6-dichloropyrimidin-5-yl)ethan-1-ol (13 g, 60.01% yield) as an off-white solid. LCMS: m/z=208 (M+1, ESI+).

### Step 2: 1-(2-amino-4,6-dichloropyrimidin-5-yl)ethan-1-one

To a solution of 1-(2-amino-4,6-dichloropyrimidin-5-yl)ethan-1-ol (13 g, 62.5 mmol) in DCM (130 mL) was added Dessmartin periodinane (53 g, 125 mmol) at 0° C., and the mixture was stirred at 0° C. for 2 hours. After the reaction was completed, the reaction mixture was quenched with water (100 mL) and extracted with EA (500 mL×2). The combined organic layer was washed with brine (100 mL), dried over anhydrous Na₂SO₄, and concentrated. The residue was purified by column chromatography to afford 1-(2-amino-4,6-dichloropyrimidin-5-yl)ethan-1-one (8.4 g, 65.24% yield) as an off-white solid. LCMS: m/z=207 (M+1, ESI+).

### Step 3: 4-chloro-3-methyl-1H-pyrazolo[3,4-d]pyrimidin-6-amine

To a solution of 1-(2-amino-4,6-dichloropyrimidin-5-yl)ethan-1-one (8.4 g, 40.8 mmol) in DCM (90 mL) was added hydrazine hydrate (99%) (1.92 mL, 61.2 mmol) at 25° C., and the mixture was stirred at 25° C. for 16 hours. After the reaction was completed, the reaction mixture was concentrated, diluted with ice water (100 mL), and the precipitated solid was filtered, washed with water (200 mL), and dried in vacuo to afford 4-chloro-3-methyl-1H-pyrazolo[3,4-d]pyrimidin-6-amine (6.7 g, 89.81% yield) as an off-white solid. LCMS: m/z=184 (M+1, ESI+).

### Step 4: 5-(6-amino-3-methyl-1H-pyrazolo[3,4-d]pyrimidin-4-yl)isophthalonitrile (Intermediate S10)

To a stirred solution of 4-chloro-3-methyl-1H-pyrazolo[3,4-d]pyrimidin-6-amine (4 g, 21.7 mmol) and 5-(4,4,5,5-tetramethyl-1,3,2-dioxaborolan-2-yl)isophthalonitrile (6.6 g, 26.1 mmol) in a mixture of 1,4-dioxane (45 mL) and water (5 mL) was added Cs₂CO₃ (21.2 g, 65.1 mmol) and degassed with argon for 10 minutes. Thereafter, Pd(PPh₃)₂Cl₂ (2.29 g, 3.26 mmol) was added, and the mixture was stirred at 100° C. for 16 hours. After the reaction was completed, the reaction mixture was filtered through a Celite bed and washed with EA (400 mL). The organic layer was washed with water (100 mL), dried over Na₂SO₄, and concentrated. The residue was purified by column chromatography to afford Intermediate S10 (1.8 g, 30.05%) as a yellow solid. LCMS: m/z=276 (M+1, ESI+).

### Preparation Example A1: 1-(bromomethyl)-3-ethynyl-5-(trifluoromethyl)benzene (Intermediate A1)

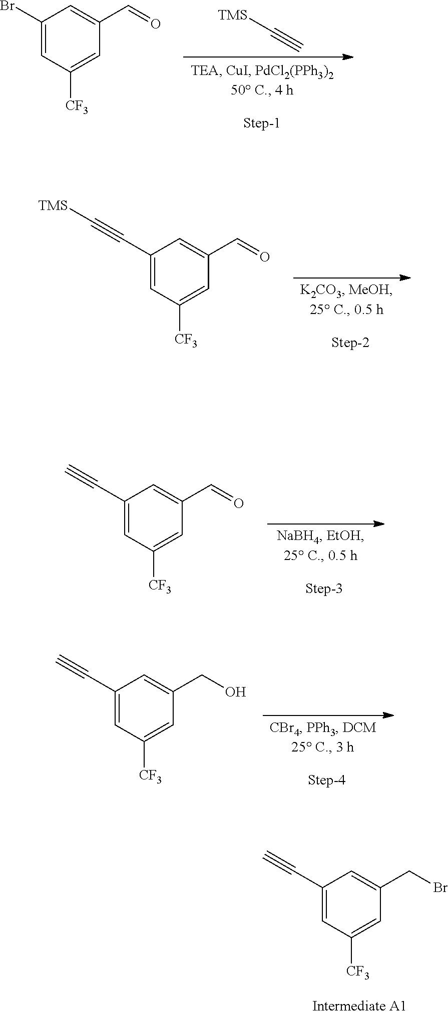

### Step 1: 3-(trifluoromethyl)-5-((trimethylsilyl)ethynyl)benzaldehyde

To a stirred solution of 3-bromo-5-(trifluoromethyl)benzaldehyde (5 g, 19.7 mmol) in TEA (40 mL) were added CuI (376 mg, 1.97 mmol) and Pd(PPh₃)₂Cl₂ (416 mg, 0.59 mmol) at 0° C. After 10 minutes, ethynyltrimethylsilane (3.37 mL, 23.7 mmol) was added dropwise, and the mixture was stirred at 50° C. for 4 hours. After the reaction was completed, the reaction mixture was quenched with water (100 mL) and extracted with EA (400 mL×2). The combined organic phase was washed with brine (50 mL×2), dried over Na₂SO₄, and concentrated. The residue was purified by column chromatography to afford 3-(trifluoromethyl)-5 -((trimethylsilyl)ethynyl)benzaldehyde (3.66 g, 68.66% yield) as a yellow liquid. ¹H NMR (400 MHz, CDCl₃) δ 10.03 (s, 1H), 8.12 (s, 1H), 8.06 (s, 1H), 7.94 (s, 1H), 0.28 (s, 9H).

### Step 2: 3-ethynyl-5-(trifluoromethyl)benzaldehyde

To a stirred solution of 3-(trifluoromethyl)-5-((trimethylsilyl)ethynyl)benzaldehyde (2.3 g, 8.51 mmol) in MeOH (30 mL) was added K₂CO₃ (1.17 g, 8.51 mmol) at 0° C., and the resulting mixture was stirred at 25° C. for 0.5 hours. After the reaction was completed, the reaction mixture was quenched with water (50 mL) and extracted with EA (350 mL×2). The combined organic phase was washed with brine (50 mL), dried over Na₂SO₄, and concentrated to afford 3-ethynyl-5-(trifluoromethyl)benzaldehyde (1.98 g, crude) as a yellow liquid. The crude product was used as it is in the next step without further purification. ¹H NMR (400 MHz, CDCl₃) δ 10.04 (s, 1H), 8.15 (m, 1H), 8.10 (m, 1H), 7.97 (m, 1H), 4.12 (d, J=7.2 Hz, 1H).

### Step 3: (3-ethynyl-5-(trifluoromethyl)phenyl)methanol

To a stirred solution of 3-ethynyl-5-(trifluoromethyl)benzaldehyde (1.98 g, 9.94 mmol) in EtOH (20 mL) was added NaBH₄ (188 mg, 4.97 mmol) at 0° C., and the mixture was stirred at 25° C. for 0.5 hours. After the reaction was completed, the reaction mixture was quenched with ice and extracted with EA (250 mL×2). The combined organic layer was washed with brine solution (40 mL), dried over Na₂SO₄, and concentrated. The residue was purified by column chromatography to afford (3-ethynyl-5-(trifluoromethyl)phenyl)methanol (1.14 g, 57.00% yield) as a yellow solid. ¹H NMR (400 MHz, DMSO-d₆) δ 7.69 (m, 3H), 5.48 (t, J=6.0 Hz, 1H), 4.58 (d, J=5.6 Hz, 2H), 4.38 (s, 1H).

### Step 4: 1-(bromomethyl)-3-ethynyl-5-(trifluoromethyl)benzene (Intermediate A1)

To a stirred solution of (3-ethynyl-5-(trifluoromethyl)phenyl)methanol (1.0 g, 5.0 mmol) in dry DCM (15 mL) were added CBr₄ (3.54 g, 10.0 mmol) and TPP (2.80 g, 10.0 mmol) at 0° C., and the mixture was stirred at 25° C. for 3 hours. After the reaction was completed, the reaction mixture was quenched with ice and extracted with EA (200 mL×2). The combined organic phase was washed with brine (40 mL), dried over Na₂SO₄, and concentrated. The residue was purified by column chromatography to afford Intermediate A1 (1.0 g, 76.04% yield) as a pale yellow solid. ¹H NMR (400 MHz, CDCl₃) δ 7.68 (d, J=6.4 Hz, 2H), 7.61 (s, 1H), 4.46 (s, 2H), 3.18 (s, 1H).

### Example 1: 5-(6-amino-1-(3-(trifluoromethyl)benzyl)-1H-pyrazolo[3,4-d]pyrimidin-4-yl)isophthalonitrile

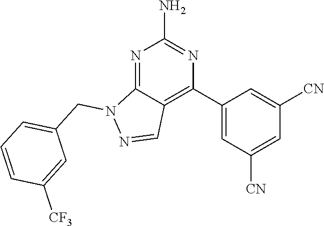 [SMILES: N#Cc1cc(C#N)cc(-c2nc(N)nc3c2cnn3Cc2cccc(C(F)(F)F)c2)c1]

To a stirred solution of Intermediate S3 (48 g, 146.4 mmol) and 5-(4,4,5,5-tetramethyl-1,3,2-dioxaborolan-2-yl)isophthalonitrile (44.6 g, 175.7 mmol) in a mixture of 1,4-dioxane (430 mL) and water (50 mL) was added K₂CO₃ (40.5 g, 293 mmol) at 25° C., and the mixture was degassed with argon for 10 minutes. Thereafter, Pd(PPh₃)₄ (33 g, 29.3 mmol) was added, and the mixture was stirred at 110° C. for 12 hours. After the reaction was completed, the solvent was evaporated, water (100 mL) was added, and the mixture was extracted with EA (500 ml×3). The combined organic layer was washed with brine (50 mL), dried over anhydrous Na₂SO₄, and concentrated. The residue was purified by column chromatography to afford the compound of Example 1 (22 g, 35.81% yield) as a pale yellow solid. ¹H NMR (400 MHz, DMSO-d₆) δ 8.82 (s, 2H), 8.69 (s, 1H), 8.60 (s, 1H), 7.67-7.56 (m, 3H), 7.47 (d, 1H), 7.22 (s, 2H), 5.58 (s, 2H); LCMS: m/z=420 (M+1, ESI+); HRMS: 420.1185 (M+1, ESI+); MR: 196° C.-200° C.

### Example 2: 5-(6-amino-1-(3-fluoro-5-(trifluoromethyl)benzyl)-1H-pyrazolo[3,4-d]pyrimidin-4-yl)isophthalonitrile

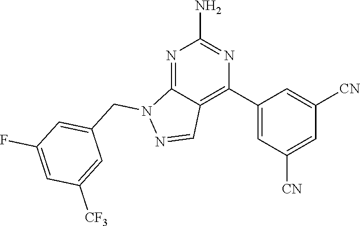 [SMILES: N#Cc1cc(C#N)cc(-c2nc(N)nc3c2cnn3Cc2cc(F)cc(C(F)(F)F)c2)c1]

To a stirred solution of Intermediate S2 (0.6 g, crude, 1.98 mmol) in DMF (10 mL) was added K₂CO₃ (545 mg, 3.96 mmol) at 0° C. and stirred for 10 minutes. Thereafter, 1-(bromomethyl)-3-fluoro-5-(trifluoromethyl)benzene (508 mg, 1.98 mmol) was added, and the resulting mixture was stirred at 25° C. for 4 hours. After the reaction was completed, the reaction mixture was quenched with ice, and the resulting precipitate was filtered and washed with water. The residue was purified by column chromatography to afford the compound of Example 2 (140 mg, 16.2%) as an off-white solid. ¹H NMR (400 MHz, DMSO-d6) δ 8.82 (s, 2H), 8.69 (s, 1H), 8.61 (s, 1H), 7.63 (d, 1H), 7.47 (s, 1H), 7.33 (d, 1H), 7.23 (s, 2H), 5.59 (s, 2H); LCMS: m/z=438 (M+1, ESI+); IRMS: 438.0567; M.R: 204° C.-208° C.

### Example 3: 3-(6-amino-1-(3-fluoro-5-(trifluoromethyl)benzyl)-1H-pyrazolo[3,4-d]pyrimidin-4-yl)-5-cyanobenzamide

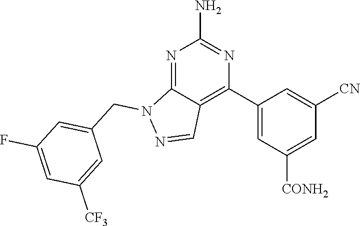 [SMILES: N#Cc1cc(C(N)=O)cc(-c2nc(N)nc3c2cnn3Cc2cc(F)cc(C(F)(F)F)c2)c1]

To a stirred solution of the compound of Example 2 (0.3 g, 0.686 mmol) in dry toluene (10 mL) were added InCl₃ (7.6 mg, 0.034 mmol) and acetaldoxime (203 mg, 3.43 mmol) at room temperature. The resulting mixture was stirred in a preheated oil bath at 70° C. for 40 minutes. After the reaction was completed, the mixture was cooled to room temperature, and the solvent was evaporated to afford a residue. The residue was purified by column chromatography to afford the compound of Example 3 (93 mg, 27.3%) as an off-white solid. ¹H NMR (400 MHz, DMSO-d₆) δ 8.84 (s, 1H), 8.65 (s, 1H), 8.49 (d, 2H), 8.33 (s, 1H), 7.78 (s, 1H), 7.63 (d, 1H), 7.48 (s, 1H), 7.33 (d, 1H), 7.20 (s, 2H), 5.59 (s, 2H); LCMS: m/z=456 (M+1, ESI+); M.R: 248° C.-252° C.

### Example 4: 5-(6-amino-1-(3-chloro-5-(trifluoromethyl)benzyl)-1H-pyrazolo[3,4-d]pyrimidin-4-yl)isophthalonitrile

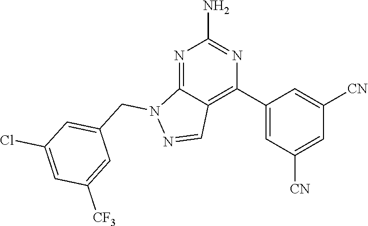 [SMILES: N#Cc1cc(C#N)cc(-c2nc(N)nc3c2cnn3Cc2cc(Cl)cc(C(F)(F)F)c2)c1]

The compound of Example 4 was obtained as an off-white solid in the same manner as in Example 2 using Intermediate S2 (0.3 g, crude, 0.988 mmol) and 1-(bromomethyl)-3-chloro-5-(trifluoromethyl)benzene (270 mg, 0.988 mmol). ¹H NMR (400 MHz, DMSO-d6) δ 8.82 (s, 2H), 8.69 (s, 1H), 8.61 (s, 1H), 7.82 (s, 1H), 7.60 (s, 1H), 7.55 (s, 1H), 7.24 (s, 2H), 5.58 (s, 2H); LCMS: m/z=454 (M+1, ESI+); HRMS: 454.0257; M.R: 214° C.-219° C.

### Example 5: 3-(6-amino-1-(3-chloro-5-(trifluoromethyl)benzyl)-1H-pyrazolo[3,4-d]pyrimidin-4-yl)-5-cyanobenzamide

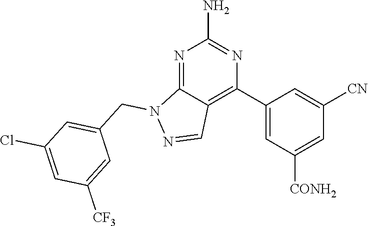 [SMILES: N#Cc1cc(C(N)=O)cc(-c2nc(N)nc3c2cnn3Cc2cc(Cl)cc(C(F)(F)F)c2)c1]

The compound of Example 5 (95 mg, 22.8%) was obtained as an off-white solid in the same manner as in Example 3 using the compound of Example 4 (0.4 g, 0.881 mmol). ¹H NMR (400 MHz, DMSO-d₆) δ 8.84 (t, 1H), 8.65 (t, 1H), 8.49 (s, 2H), 8.33 (s, 1H), 7.80 (d, 2H), 7.58 (s, 2H), 7.22 (s, 2H), 5.59 (s, 2H); LCMS: m/z=472 (M+1, ESI+).

### Example 6: 5-(6-amino-1-(3-bromo-5-(trifluoromethyl)benzyl)-1H-pyrazolo[3,4-d]pyrimidin-4-yl)isophthalonitrile

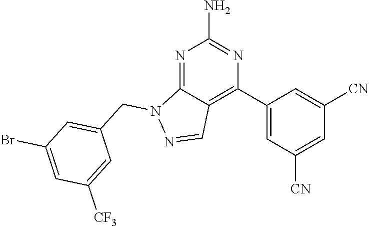 [SMILES: N#Cc1cc(C#N)cc(-c2nc(N)nc3c2cnn3Cc2cc(Br)cc(C(F)(F)F)c2)c1]

To a stirred solution of Intermediate S2 (0.6 g, crude, 1.97 mmol) in DMF (10 mL) was added K₂CO₃ (0.545 g, 3.95 mmol) at 0° C. and stirred for 10 minutes. Thereafter, 1-bromo-3-(bromomethyl)-5-(trifluoromethyl)benzene (0.626 g, 1.97 mmol) was added, and the mixture was stirred at 25° C. for 2 hours. After the reaction was completed, the reaction mixture was quenched with ice and extracted with EA (75 mL×2). The combined organic layer was washed with ice water (20 mL×2) and brine (20 mL), dried over Na₂SO₄, and concentrated. The residue was purified by column chromatography to afford the compound of Example 6 (160 mg, 16.29% yield) as an off-white solid. ¹NMR (400 MHz, DMSO-d₆) δ 8.81 (s, 2H), 8.68 (s, 1H), 8.60 (s, 1H), 7.91 (s, 1H), 7.70 (s, 1H), 7.63 (s, 1H), 7.23 (s, 2H), 5.58 (s, 2H); LCMS: m/z=498 (M+1, ESI+); MR: 214° C.-219° C.

### Example 7: 3-(6-amino-1-(3-bromo-5-(trifluoromethyl)benzyl)-1H-pyrazolo[3,4-d]pyrimidin-4-yl)-5-cyanobenzamide

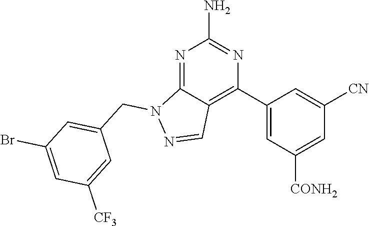 [SMILES: N#Cc1cc(C(N)=O)cc(-c2nc(N)nc3c2cnn3Cc2cc(Br)cc(C(F)(F)F)c2)c1]

The compound of Example 7 (75 mg, 20.71% yield) was obtained as an off-white solid in the same manner as in Example 3 using the compound of Example 6 (0.350 g, 0.70 mmol). ¹NMR (400 MHz, DMSO-d₆) δ 8.85 (s, 1H), 8.65 (s, 1H), 8.49 (d, 2H), 8.33 (s, 1H), 7.92 (s, 1H), 7.79 (s, 1H), 7.71 (s, 1H), 7.64 (s, 1H), 7.22 (s, 2H), 5.58 (s, 2H); LCMS: m/z=516 (M+1, ESI+).

### Example 8: 5-(6-amino-1-(3-iodo-5-(trifluoromethyl)benzyl)-1H-pyrazolo[3,4-d]pyrimidin-4-yl)isophthalonitrile

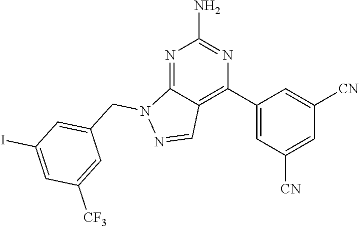 [SMILES: N#Cc1cc(C#N)cc(-c2nc(N)nc3c2cnn3Cc2cc(I)cc(C(F)(F)F)c2)c1]

The compound of Example 8 (55 mg, 5%) was obtained as an off-white solid in the same manner as in Example 2 using Intermediate S2 (0.6 g, crude, 1.97 mmol) and 1-(bromomethyl)-3-iodo-5-(trifluoromethyl)benzene (717 mg, 1.97 mmol). ¹H NMR (400 MHz, DMSO-d₆) δ 8.82 (s, 2H), 8.69 (s, 1H), 8.60 (s, 1H), 8.02 (s, 1H), 7.88 (s, 1H), 7.61 (s, 1H), 7.24 (s, 2H), 5.54 (s, 2H); LCMS: m/z=546 (M+1, ESI+); MR: 200° C.-206° C.

### Example 9: 3-(6-amino-1-(3-iodo-5-(trifluoromethyl)benzyl)-1H-pyrazolo[3,4-d]pyrimidin-4-yl)-5-cyanobenzamide

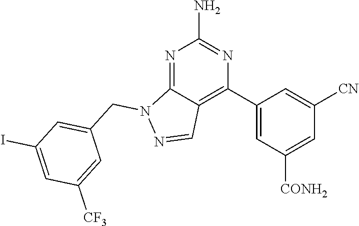 [SMILES: N#Cc1cc(C(N)=O)cc(-c2nc(N)nc3c2cnn3Cc2cc(I)cc(C(F)(F)F)c2)c1]

The compound of Example 9 (50 mg, 24%) was obtained as an off-white solid in the same manner as in Example 3 using the compound of Example 8 (0.2 g, 0.366 mmol). ¹H NMR (400 MHz, DMSO-d6) δ 8.85 (s, 1H), 8.66 (s, 1H), 8.49 (d, 2H), 8.34 (s, 1H), 8.02 (s, 1H), 7.89 (s, 1H), 7.79 (s, 1H), 7.62 (s, 1H), 7.22 (s, 2H), 5.55 (s, 2H); LCMS: m/z=562

### Example 10: 5-(6-amino-1-(3,5-bis(trifluoromethyl)benzyl)-1H-pyrazolo[3,4-d]pyrimidin-4-yl)isophthalonitrile

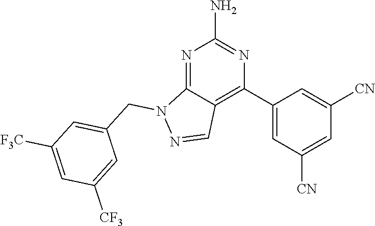 [SMILES: N#Cc1cc(C#N)cc(-c2nc(N)nc3c2cnn3Cc2cc(C(F)(F)F)cc(C(F)(F)F)c2)c1]

The compound of Example 10 (170 mg, 17.6%) was obtained as an off-white solid in the same manner as in Example 2 using Intermediate S2 (0.6 g, crude, 1.98 mmol) and 1-(bromomethyl)-3,5-bis(trifluoromethyl)benzene (608 mg, 1.98 mmol). ¹H NMR (400 MHz, DMSO-d₆) δ 8.82 (s, 2H), 8.68 (s, 1H), 8.62 (s, 1H), 8.06 (s, 1H), 7.92 (s, 2H), 7.23 (s, 2H), 5.68 (s, 2H); LCMS: m/z=488 (M+1, ESI+); M.R: 220° C.-226° C.

### Example 11: 3-(6-amino-1-(3,5-bis(trifluoromethyl)benzyl)-1H-pyrazolo[3,4-d]pyrimidin-4-yl)-5-cyanobenzamide

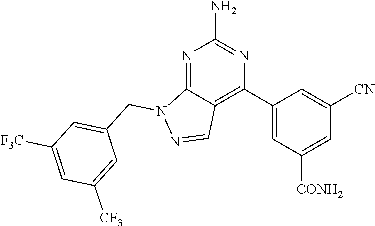 [SMILES: N#Cc1cc(C(N)=O)cc(-c2nc(N)nc3c2cnn3Cc2cc(C(F)(F)F)cc(C(F)(F)F)c2)c1]

The compound of Example 11 (85 mg, 27.33% yield) was obtained as an off-white solid in the same manner as in Example 3 using the compound of Example 10 (0.3 g, 0.6 mmol). ¹H NMR (400 MHz, DMSO-d₆) δ 8.85 (s, 1H), 8.66 (s, 1H), 8.50 (d, 2H), 8.33 (s, 1H), 8.08 (s, 1H), 7.94 (s, 2H), 7.79 (s, 1H), 7.22 (s, 2H), 5.68 (s, 2H); LCMS: m/z=506 (M+1, ESI+).

### Examples 12 to 40

The compounds of Examples 12 to 40 were obtained in the same manner as in Example 6 using the starting materials listed in Table A below. In Examples 20, 21, 22, 23, 28, 30, and 32 to 40, Cs₂CO₃ was used instead of K₂CO₃. Stirring time and purification method, etc. were modified as appropriate.

TABLE A

| Example | Chemical structure | IUPAC Name | Starting material |
|---|---|---|---|
| 12 | 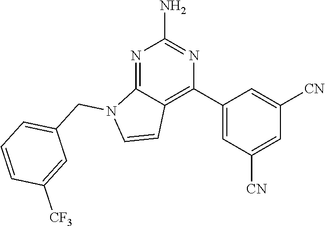 [SMILES: N#Cc1cc(C#N)cc(-c2nc(N)nc3c2ccn3Cc2cccc(C(F)(F)F)c2)c1] | 5-(2-amino-7-(3- (trifluoromethyl)benzyl)- 7H-pyrrolo[2,3- d]pyrimidin-4- yl)isophthalonitrile | Intermediate S4 and 1- (bromomethyl)-3- (trifluoromethyl) benzene |
| 13 | 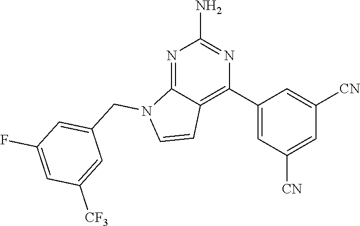 [SMILES: N#Cc1cc(C#N)cc(-c2nc(N)nc3c2ccn3Cc2cc(F)cc(C(F)(F)F)c2)c1] | 5-(2-amino-7-(3-fluoro-5- (trifluoromethyl)benzyl)- 7H-pyrrolo[2,3- d]pyrimidin-4- yl)isophthalonitrile | Intermediate S4 and 1- (bromomethyl)-3- fluoro-5- (trifluoromethyl) benzene |
| 14 | 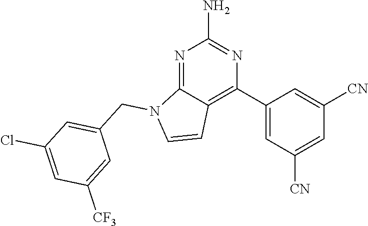 [SMILES: N#Cc1cc(C#N)cc(-c2nc(N)nc3c2ccn3Cc2cc(Cl)cc(C(F)(F)F)c2)c1] | 5-(2-amino-7-(3-chloro-5- (trifluoromethyl)benzyl)- 7H-pyrrolo[2,3- d]pyrimidin-4- yl)isophthalonitrile | Intermediate S4 and 1- (bromomethyl)-3- chloro-5- (trifluoromethyl) benzene |
| 15 | 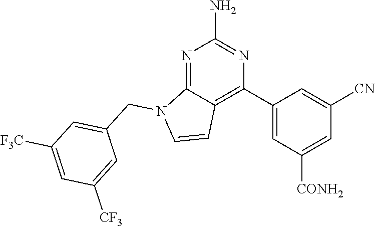 [SMILES: N#Cc1cc(C(N)=O)cc(-c2nc(N)nc3c2ccn3Cc2cc(C(F)(F)F)cc(C(F)(F)F)c2)c1] | 5-(2-amino-7-(3,5- bis(trifluoromethyl)benzyl)- 7H-pyrrolo[2,3- d]pyrimidin-4- yl)isophthalonitrile | Intermediate S4 and 1- (bromomethyl)-3,5- bis(trifluoromethyl) benzene |
| 16 | 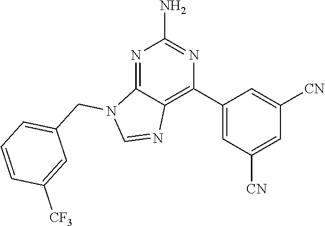 [SMILES: N#Cc1cc(C#N)cc(-c2nc(N)nc3c2ncn3Cc2cccc(C(F)(F)F)c2)c1] | 5-(2-amino-9-(3- (trifluoromethyl)benzyl)- 9H-purin-6- yl)isophthalonitrile | Intermediate S5 and 1- (bromomethyl)-3- (trifluoromethyl) benzene |
| 17 | 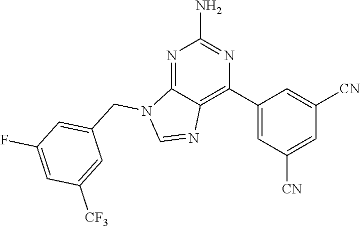 [SMILES: N#Cc1cc(C#N)cc(-c2nc(N)nc3c2ncn3Cc2cc(F)cc(C(F)(F)F)c2)c1] | 5-(2-amino-9-(3-fluoro-5- (trifluoromethyl)benzyl)- 9H-purin-6- yl)isophthalonitrile | Intermediate S5 and 1- (bromomethyl)-3- fluoro-5- (trifluoromethyl) benzene |
| 18 | 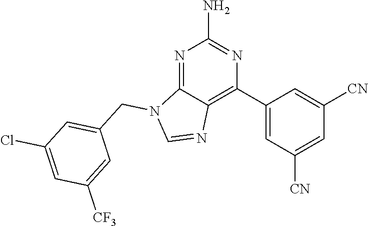 [SMILES: N#Cc1cc(C#N)cc(-c2nc(N)nc3c2ncn3Cc2cc(Cl)cc(C(F)(F)F)c2)c1] | 5-(2-amino-9-(3-chloro-5- (trifluoromethyl)benzyl)- 9H-purin-6- yl)isophthalonitrile | Intermediate S5 and 1- (bromomethyl)-3- chloro-5- (trifluoromethyl) benzene |
| 19 | 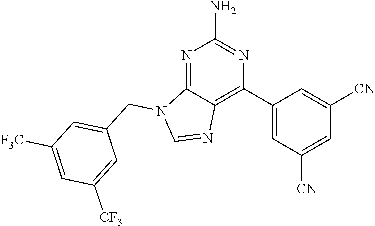 [SMILES: N#Cc1cc(C#N)cc(-c2nc(N)nc3c2ncn3Cc2cc(C(F)(F)F)cc(C(F)(F)F)c2)c1] | 5-(2-amino-9-(3,5- bis(trifluoromethyl)benzyl)- 9H-purin-6- yl)isophthalonitrile | Intermediate S5 and 1- (bromomethyl)-3,5- bis(trifluoromethyl) benzene |
| 20 | 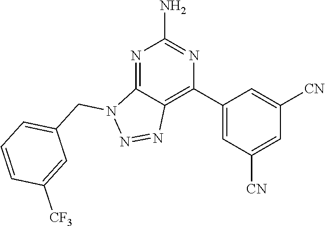 [SMILES: N#Cc1cc(C#N)cc(-c2nc(N)nc3c2nnn3Cc2cccc(C(F)(F)F)c2)c1] | 5-(5-amino-3-(3- (trifluoromethyl)benzyl)- 3H-[1,2,3]triazolo[4,5- d]pyrimidin-7- yl)isophthalonitrile | Intermediate S6 and 1- (bromomethyl)-3- (trifluoromethyl) benzene |
| 21 | 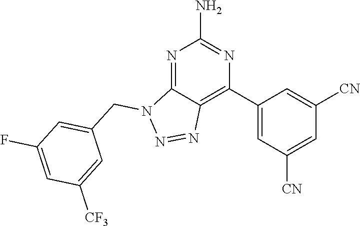 [SMILES: N#Cc1cc(C#N)cc(-c2nc(N)nc3c2nnn3Cc2cc(F)cc(C(F)(F)F)c2)c1] | 5-(5-amino-3-(3-fluoro-5- (trifluoromethyl)benzyl)- 3H-[1,2,3]triazolo[4,5- d]pyrimidin-7- yl)isophthalonitrile | Intermediate S6 and 1- (bromomethyl)-3- fluoro-5- (trifluoromethyl) benzene |
| 22 | 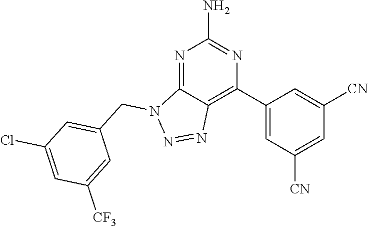 [SMILES: N#Cc1cc(C#N)cc(-c2nc(N)nc3c2nnn3Cc2cc(Cl)cc(C(F)(F)F)c2)c1] | 5-(5-amino-3-(3-chloro-5- (trifluoromethyl)benzyl)- 3H-[1,2,3]triazolo[4,5- d]pyrimidin-7- yl)isophthalonitrile | Intermediate S6 and 1- (bromomethyl)-3- chloro-5- (trifluoromethyl) benzene |
| 23 | 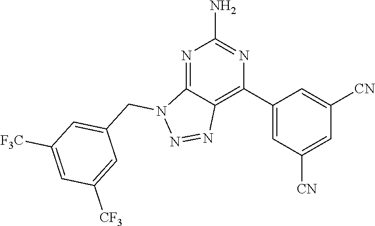 [SMILES: N#Cc1cc(C#N)cc(-c2nc(N)nc3c2nnn3Cc2cc(C(F)(F)F)cc(C(F)(F)F)c2)c1] | 5-(5-amino-3-(3,5- bis(trifluoromethyl)benzyl)- 3H-[1,2,3]triazolo[4,5- d]pyrimidin-7- yl)isophthalonitrile | Intermediate S6 and 1- (bromomethyl)-3,5- bis(trifluoromethyl) benzene |
| 24 | 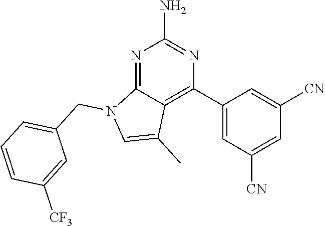 [SMILES: Cc1cn(Cc2cccc(C(F)(F)F)c2)c2nc(N)nc(-c3cc(C#N)cc(C#N)c3)c12] | 5-(2-amino-5-methyl-7-(3- (trifluoromethyl)benzyl)- 7H-pyrrolo[2,3- d]pyrimidin-4- yl)isophthalonitrile | Intermediate S7 and 1- (bromomethyl)-3- (trifluoromethyl) benzene |
| 25 | 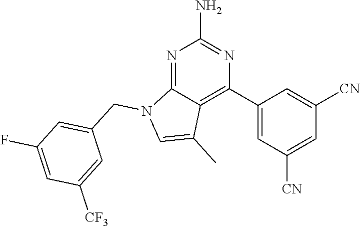 [SMILES: Cc1cn(Cc2cc(F)cc(C(F)(F)F)c2)c2nc(N)nc(-c3cc(C#N)cc(C#N)c3)c12] | 5-(2-amino-7-(3-fluoro-5- (trifluoromethyl)benzyl)-5- methyl-7H-pyrrolo[2,3- d]pyrimidin-4- yl)isophthalonitrile | Intermediate S7 and 1- (bromomethyl)-3- fluoro-5- (trifluoromethyl) benzene |
| 26 | 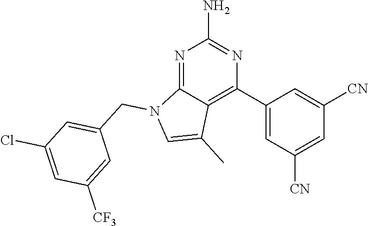 [SMILES: Cc1cn(Cc2cc(Cl)cc(C(F)(F)F)c2)c2nc(N)nc(-c3cc(C#N)cc(C#N)c3)c12] | 5-(2-amino-7-(3-chloro-5- (trifluoromethyl)benzyl)-5- methyl-7H-pyrrolo[2,3- d]pyrimidin-4- yl)isophthalonitrile | Intermediate S7 and 1- (bromomethyl)-3- chloro-5- (trifluoromethyl) benzene |
| 27 | 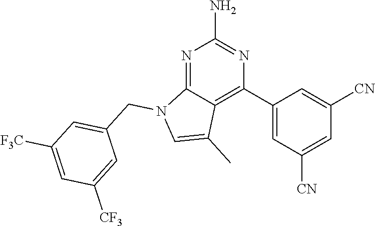 [SMILES: Cc1cn(Cc2cc(C(F)(F)F)cc(C(F)(F)F)c2)c2nc(N)nc(-c3cc(C#N)cc(C#N)c3)c12] | 5-(2-amino-7-(3,5- bis(trifluoromethyl)benzyl)- 5-methyl-7H-pyrrolo[2,3- d]pyrimidin-4- yl)isophthalonitrile | Intermediate S7 and 1- (bromomethyl)-3,5- bis(trifluoromethyl) benzene |
| 28 | 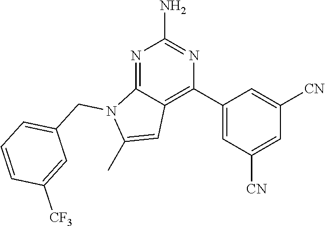 [SMILES: Cc1cc2c(-c3cc(C#N)cc(C#N)c3)nc(N)nc2n1Cc1cccc(C(F)(F)F)c1] | 5-(2-amino-6-methyl-7-(3- (trifluoromethyl)benzyl)- 7H-pyrrolo[2,3- d]pyrimidin-4- yl)isophthalonitrile | Intermediate S8 and 1- (bromomethyl)-3- (trifluoromethyl) benzene |
| 29 | 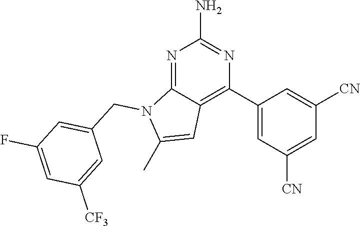 [SMILES: Cc1cc2c(-c3cc(C#N)cc(C#N)c3)nc(N)nc2n1Cc1cc(F)cc(C(F)(F)F)c1] | 5-(2-amino-7-(3-fluoro-5- (trifluoromethyl)benzyl)-6- methyl-7H-pyrrolo[2,3- d]pyrimidin-4- yl)isophthalonitrile | Intermediate S8 and 1- (bromomethyl)-3- fluoro-5- (trifluoromethyl) benzene |
| 30 | 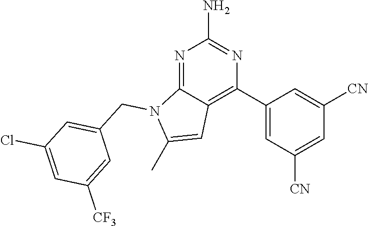 [SMILES: Cc1cc2c(-c3cc(C#N)cc(C#N)c3)nc(N)nc2n1Cc1cc(Cl)cc(C(F)(F)F)c1] | 5-(2-amino-7-(3-chloro-5- (trifluoromethyl)benzyl)-6- methyl-7H-pyrrolo[2,3- d]pyrimidin-4- yl)isophthalonitrile | Intermediate S8 and 1- (bromomethyl)-3- chloro-5- (trifluoromethyl) benzene |
| 31 | 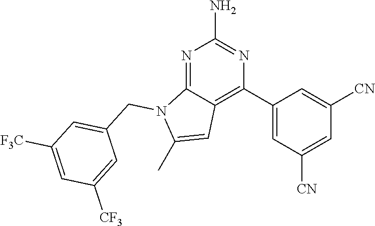 [SMILES: Cc1cc2c(-c3cc(C#N)cc(C#N)c3)nc(N)nc2n1Cc1cc(C(F)(F)F)cc(C(F)(F)F)c1] | 5-(2-amino-7-(3,5- bis(trifluoromethyl)benzyl)- 6-methyl-7H-pyrrolo[2,3- d]pyrimidin-4- yl)isophthalonitrile | Intermediate S8 and 1- (bromomethyl)-3,5- bis(trifluoromethyl) benzene |
| 32 | 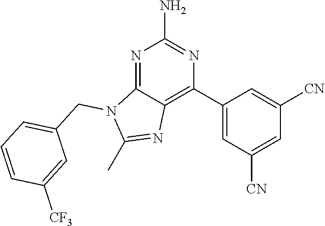 [SMILES: Cc1nc2c(-c3cc(C#N)cc(C#N)c3)nc(N)nc2n1Cc1cccc(C(F)(F)F)c1] | 5-(2-amino-8-methyl-9-(3- (trifluoromethyl)benzyl)- 9H-purin-6- yl)isophthalonitrile | Intermediate S9 and 1- (bromomethyl)-3- (trifluoromethyl) benzene |
| 33 | 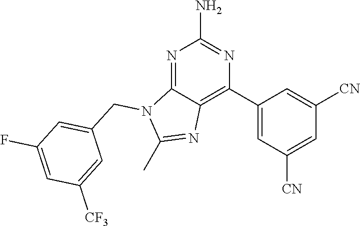 [SMILES: Cc1nc2c(-c3cc(C#N)cc(C#N)c3)nc(N)nc2n1Cc1cc(F)cc(C(F)(F)F)c1] | 5-(2-amino-9-(3-fluoro-5- (trifluoromethyl)benzyl)-8- methyl-9H-purin-6- yl)isophthalonitrile | Intermediate S9 and 2- (bromomethyl)-1- fluoro-4- (trifluoromethyl) benzene |
| 34 | 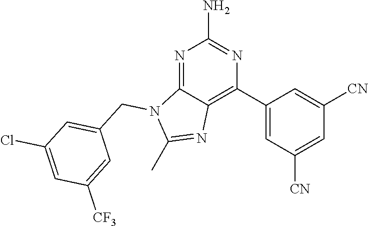 [SMILES: Cc1nc2c(-c3cc(C#N)cc(C#N)c3)nc(N)nc2n1Cc1cc(Cl)cc(C(F)(F)F)c1] | 5-(2-amino-9-(3-chloro-5- (trifluoromethyl)benzyl)-8- methyl-9H-purin-6- yl)isophthalonitrile | Intermediate S9 and 1- (bromomethyl)-3- chloro-5- (trifluoromethyl) benzene |
| 35 | 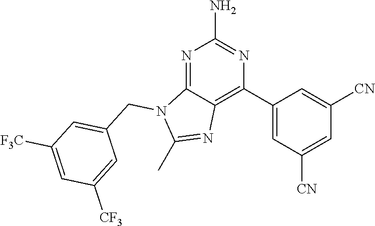 [SMILES: Cc1nc2c(-c3cc(C#N)cc(C#N)c3)nc(N)nc2n1Cc1cc(C(F)(F)F)cc(C(F)(F)F)c1] | 5-(2-amino-9-(3,5- bis(trifluoromethyl)benzyl)- 8-methyl-9H-purin-6- yl)isophthalonitrile | Intermediate S9 and 1- (bromomethyl)-3,5- bis(trifluoromethyl) benzene |
| 36 | 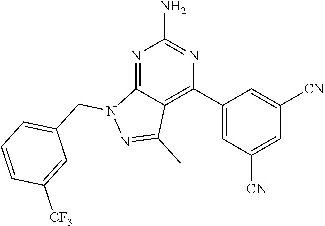 [SMILES: Cc1nn(Cc2cccc(C(F)(F)F)c2)c2nc(N)nc(-c3cc(C#N)cc(C#N)c3)c12] | 5-(6-amino-3-methyl-1-(3- (trifluoromethyl)benzyl)- 1H-pyrazolo[3,4- d]pyrimidin-4- yl)isophthalonitrile | Intermediate S10 and 1-(bromomethyl)-3- (trifluoromethyl) benzene |
| 37 | 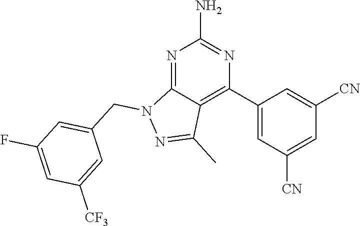 [SMILES: Cc1nn(Cc2cc(F)cc(C(F)(F)F)c2)c2nc(N)nc(-c3cc(C#N)cc(C#N)c3)c12] | 5-(6-amino-1-(3-fluoro-5- (trifluoromethyl)benzyl)-3- methyl-1H-pyrazolo[3,4- d]pyrimidin-4- yl)isophthalonitrile | Intermediate S10 and 1-(bromomethyl)-3- fluoro-5- (trifluoromethyl) benzene |
| 38 | 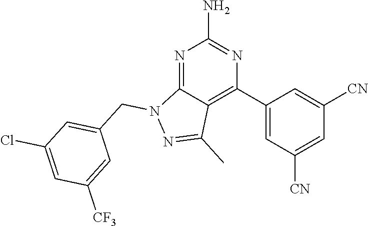 [SMILES: Cc1nn(Cc2cc(Cl)cc(C(F)(F)F)c2)c2nc(N)nc(-c3cc(C#N)cc(C#N)c3)c12] | 5-(6-amino-1-(3-chloro-5- (trifluoromethyl)benzyl)-3- methyl-1H-pyrazolo[3,4- d]pyrimidin-4- yl)isophthalonitrile | Intermediate S10 and 1-(bromomethyl)-3- chloro-5- (trifluoromethyl) benzene |
| 39 | 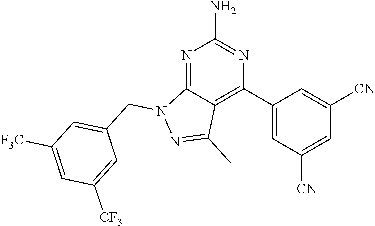 [SMILES: Cc1nn(Cc2cc(C(F)(F)F)cc(C(F)(F)F)c2)c2nc(N)nc(-c3cc(C#N)cc(C#N)c3)c12] | 5-(6-amino-1-(3,5- bis(trifluoromethyl)benzyl)- 3-methyl-1H-pyrazolo[3,4- d]pyrimidin-4- yl)isophthalonitrile | Intermediate S10 and 1-(bromomethyl)-3,5- bis(trifluoromethyl) benzene |
| 40 | 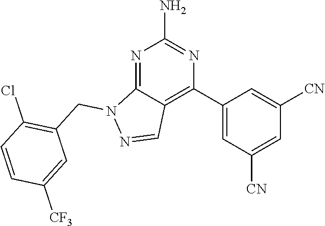 [SMILES: N#Cc1cc(C#N)cc(-c2nc(N)nc3c2cnn3Cc2cc(C(F)(F)F)ccc2Cl)c1] | 5-(6-amino-1-(2-chloro-5- (trifluoromethyl)benzyl)- 1H-pyrazolo[3,4- d]pyrimidin-4- yl)isophthalonitrile | Intermediate S2 and 2- (bromomethyl)-1- chloro-4- (trifluoromethyl) benzene |

TABLE B

| Example | Analytical Data |
|---|---|
| 12 | ¹H NMR (400 MHz, DMSO-d₆) δ 8.71 (s, 2H), 8.61 (s, 1H), 7.64 (d, 2H), 7.57 |
| | (t, 1H), 7.46 (d, 1H), 7.38 (d, 1H), 6.88 (d, 1H), 6.55 (s, 2H), 5.42 (s, 2H); LCMS: |
| | m/z = 419 (M + 1, ESI+); MR: 208° C.-212° C. |
| 13 | ¹H NMR (400 MHz, DMSO-d6) δ 8.71 (d, 2H), 8.61 (t, 1H), 7.62 (d, 1H), 7.49 |
| | (s, 1H), 7.40 (d, 1H), 7.34 (d, 1H), 6.90 (d, 1H), 6.57 (s, 2H), 5.42 (s, 2H); LCMS: |
| | m/z = 437 (M + 1, ESI+); MR: 206° C.-210° C. |
| 14 | ¹H NMR (400 MHz, DMSO-d6) δ 8.71 (d, 2H), 8.61 (s, 1H), 7.80 (s, 1H), 7.61 |
| | (s, 1H), 7.55 (s, 1H), 7.41 (d, 1H), 6.90 (d, 1H), 6.58 (s, 2H), 5.42 (s, 2H); LCMS: |
| | m/z = 453 (M + 1, ESI+); MR: 187° C.-191° C. |
| 15 | ¹H NMR (400 MHz, DMSO-d6) δ 8.72 (d, 2H), 8.61 (t, 1H), 8.06 (s, 1H), 7.93 |
| | (s, 2H), 7.43 (d, 1H), 6.91 (d, 1H), 6.57 (s, 2H), 5.51 (s, 2H); LCMS: m/z = 487 |
| | (M + 1, ESI+). |
| 16 | ¹H NMR (400 MHz, DMSO-d6) δ 9.31 (d, 2H), 8.63 (s, 1H), 8.44 (s, 1H), 7.73 |
| | (s, 1H), 7.68 (d, 1H), 7.61-7.53 (m, 2H) 6.84 (s, 2H), 5.47 (s, 2H); LCMS: |
| | m/z = 420 (M + 1, ESI+); MR: 236° C.-241° C. |
| 17 | ¹HNMR (400 MHz, DMSO-d6) δ 9.31 (d, 2H), 8.64 (t, 1H), 8.44 (s, 1H), 7.66 (d, |
| | 1H), 7.59 (s, 1H), 7.47 (d, 1H), 6.85 (d, 2H), 5.47 (s, 2H); LCMS: m/z = 438 |
| | (M + 1, ESI+); MR: 249° C.-252° C. |
| 18 | ¹HNMR (400 MHz, DMSO-d6) δ 9.29 (d, 2H), 8.62 (d, 1H), 8.43 (s, 1H), 7.82 |
| | (s, 1H), 7.69 (d, 2H), 6.84 (s, 2H), 5.46 (s, 2H); LCMS: m/z = 454 (M + 1, ESI+); |
| | MR: 224° C.-230° C. |
| 19 | ¹HNMR (400 MHz, DMSO-d6) δ 9.29 (d, 2H), 8.61 (t, 1H), 8.45 (s, 1H), 8.07 (s, |
| | 1H), 8.04 (s, 2H), 6.83 (s, 2H), 5.56 (s, 2H); LCMS: m/z = 488 (M + 1, ESI+). |
| 20 | ¹HNMR (400 MHz, DMSO-d6) δ 9.26 (s, 2H), 8.77 (s, 1H), 7.76 (s, 1H), 7.71 (d, |
| | 1H), 7.62-7.54 (m, 4H), 5.85 (s, 2H); LCMS: m/z = 421 (M + 1, ESI+); MR: |
| | 200° C.-206° C. |
| 21 | ¹HNMR (400 MHz, DMSO-d6) δ 9.26 (d, 2H), 8.77 (t, 1H), 7.69 (d, 1H), 7.62 |
| | (s, 3H), 7.46 (d, 1H), 5.87 (s, 2H); LCMS: m/z = 439 (M + 1, ESI+); MR: |
| | 218° C.-224° C. |
| 22 | ¹HNMR (400 MHz, DMSO-d6) δ 9.25 (s, 2H), 8.77 (s, 1H), 7.87 (s, 1H), 7.73- |
| | 7.63 (m, 4H), 5.87 (s, 2H); LCMS: m/z = 455 (M + 1, ESI+); MR: 234° C.-238° C. |
| 23 | ¹HNMR (400 MHz, DMSO-d6) δ 9.26 (d, 2H), 8.77 (t, 1H), 8.13 (s, 1H), 8.07 (s, |
| | 2H), 7.64 (s, 2H), 5.96 (s, 2H); LCMS: m/z = 489 (M + 1, ESI+); MR: 210° C.- |
| | 216° C. |
| 24 | ¹H NMR (400 MHz, DMSO-d₆) δ 8.60 (s, 1H), 8.42 (s, 2H), 7.68-7.64 (m, 2H), |
| | 7.59-7.55 (m, 1H), 7.47 (d, 1H), 7.05 (s, 1H), 6.48 (s, 2H), 5.34 (s, 2H), 1.86 (s, |
| | 3H); LCMS: m/z = 433 (M + 1, ESI+); MR: 193° C.-198° C. |
| 25 | ¹H NMR (400 MHz, DMSO-d₆) δ 8.60 (t, 1H), 8.43 (d, 2H), 7.64-7.61 (m, 1H), |
| | 7.54 (s, 1H), 7.34-7.32 (s, 1H), 7.07 (d, 1H), 6.51 (s, 2H), 5.35 (s, 2H), 1.86 (s, |
| | 3H); LCMS: m/z = 451 (M + 1, ESI+); MR: 202° C.-206° C. |
| 26 | ¹H NMR (400 MHz, DMSO-d6) δ 8.60 (s, 1H), 8.43 (d, 2H), 7.81 (s, 1H), 7.66 |
| | (s, 1H), 7.56 (s, 1H), 7.07 (s, 1H), 6.51 (s, 2H), 5.34 (s, 2H), 1.86 (s, 3H); LCMS: |
| | m/z = 467 (M + 1, ESI+); MR: 222° C.-226° C. |
| 27 | ¹H NMR (400 MHz, DMSO-d₆) δ 8.61 (s, 1H), 8.44 (d, 2H), 8.07 (s, 1H), 7.99 |
| | (s, 2H), 7.11 (s, 1H), 6.53 (s, 2H), 5.44 (s, 2H), 1.88 (s, 3H); LCMS: m/z = 501 |
| | (M + 1, ESI+); MR: 222° C.-226° C. |
| 28 | ¹H NMR (400 MHz, DMSO-d6) δ 8.73 (d, 2H), 8.60 (t, 1H), 7.65 (d, 1H), 7.58- |
| | 7.54 (m, 2H), 7.28 (d, 1H), 6.73 (s, 1H), 6.45 (s, 2H), 5.46 (s, 2H), 2.28 (s, 3H); |
| | LCMS: m/z = 433 (M + 1, ESI+); MR: 241° C.-246° C. |
| 29 | ¹H NMR (400 MHz, DMSO-d6) δ 8.73 (s, 2H), 8.60 (s, 1H), 7.62 (d, 1H), 7.37 |
| | (s, 1H), 7.16 (d, 1H), 6.74 (s, 1H), 6.46 (s, 2H), 5.46 (s, 2H), 2.29 (s, 3H); LCMS: |
| | m/z = 451 (M + 1, ESI+). |
| 30 | ¹H NMR (400 MHz, DMSO-d6) δ 8.73 (s, 2H), 8.60 (s, 1H), 7.80 (s, 1H), 7.48 |
| | (s, 1H), 7.37 (s, 1H), 6.74 (s, 1H), 6.47 (s, 2H), 5.45 (s, 2H), 2.29 (s, 3H); LCMS: |
| | m/z = 467 (M + 1, ESI+); MR: 220° C.-226° C. |
| 31 | ¹H NMR (400 MHz, DMSO-d6) δ 8.73 (s, 2H), 8.60 (s, 1H), 8.06 (s, 1H), 7.77 |
| | (s, 2H), 6.76 (s, 1H), 6.48 (s, 2H), 5.55 (s, 2H), 2.29 (s, 3H); LCMS: m/z = 501 |
| | (M + 1, ESI+). |
| 32 | ¹H NMR (400 MHz, Acetone-d₆) δ 9.46 (d, 2H), 8.40 (t, 1H), 7.73 (s, 1H), 7.69- |
| | 7.55 (m, 3H), 6.18 (s, 2H), 5.55 (s, 2H), 2.57 (s, 3H); LCMS: m/z = 434 (M + 1, |
| | ESI+); MR: not melt up to 297° C. |
| 33 | ¹H NMR (400 MHz, Acetone-d₆) δ 9.32 (d, 2H), 8.27 (t, 1H), 7.47 (s, 1H), 7.35 |
| | (d, 1H), 7.27 (d, 1H), 6.07 (s, 2H), 5.44 (s, 2H), 2.46 (s, 3H); LCMS: m/z = 452 |
| | (M + 1, ESI+); MR: 281° C.-284° C. |
| 34 | ¹H NMR (400 MHz, CDCl₃) δ 9.39 (d, 2H), 8.00 (t, 1H), 7.58 (s, 1H), 7.39 (s, |
| | 1H), 7.31 (s, 1H), 5.33 (s, 2H), 5.08 (s, 2H) 2.54 (s, 3H); LCMS: m/z = 468 (M + 1, |
| | ESI+); MR: not melt up to 297 ° C. |
| 35 | ¹H NMR (400 MHz, Acetone-d₆) δ 9.32 (d, 2H), 8.28 (t, 1H), 7.90 (s, 3H), 6.08 |
| | (s, 2H), 5.54 (s, 2H), 2.48 (s, 3H) ; LCMS: m/z = 500 (M + 1, ESI+); MR: not melt |
| | up to 297° C. |
| 36 | ¹H NMR (400 MHz, DMSO-d6) δ 8.68 (s, 1H), 8.54 (d, 2H), 7.67-7.66 (m, 2H), |
| | 7.58 (t, 1H), 7.47 (d, 1H), 7.15 (s, 2H), 5.48 (s, 2H), 2.15 (s, 3H); LCMS: m/z = |
| | 434 (M + 1, ESI+); MR: 183ºC-188° C. |
| 37 | ¹H NMR (400 MHz, DMSO-d6) δ 8.68 (s, 1H), 8.54 (d, 2H), 7.64 (d, 1H), 7.53 |
| | (s, 1H), 7.31 (d, 1H), 7.17 (s, 2H), 5.50 (s, 2H), 2.15 (s, 3H); LCMS: m/z = 452 |
| | (M + 1, ESI+); MR: 206° C.-212° C. |
| 38 | ¹H NMR (400 MHz, DMSO-d6) δ 8.69 (t, 1H), 8.55 (d, 2H), 7.83 (s, 1H), 7.65 |
| | (s, 1H), 7.57 (s, 1H), 7.19 (s, 2H), 5.50 (s, 2H), 2.16 (s, 3H); LCMS: m/z = 466 |
| | (M-1, ESI−); MR: 192° C.-196° C. |
| 39 | ¹H NMR (400 MHz, DMSO-d6) δ 8.68 (d, 1H), 8.55 (d, 2H), 8.08 (s, 1H), 7.96 |
| | (s, 2H), 7.19 (s, 2H), 5.59 (s, 2H), 2.15 (s, 3H); LCMS: m/z = 502 (M + 1, ESI+); |
| | MR: 194° C.-198° C. |
| 40 | ¹H NMR (400 MHz, DMSO-d₆) δ 8.82 (d, 2H), 8.70 (s, 1H), 8.61 (s, 1H), 7.79- |
| | 7.74 (m, 2H), 7.40 (s, 1H), 7.25 (s, 2H), 5.61 (s, 2H); MS: m/z = 454 (M + 1, |
| | ESI+); MR: 208° C.-214° C. |

### Example 41: 5-(6-amino-1-(4-chloro-3-(trifluoromethyl)benzyl)-1H-pyrazolo[3,4-d]pyrimidin-4-yl)isophthalonitrile

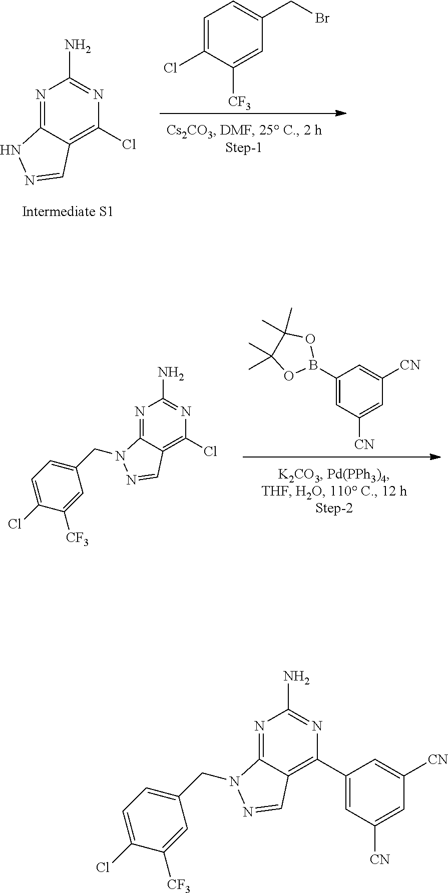 [SMILES: Nc1nc(Cl)c2cn[nH]c2n1 ; FC(F)(F)c1cc(CBr)ccc1Cl ; Nc1nc(Cl)c2cnn(Cc3ccc(Cl)c(C(F)(F)F)c3)c2n1 ; CC1(C)OB(c2cc(C#N)cc(C#N)c2)OC1(C)C ; N#Cc1cc(C#N)cc(-c2nc(N)nc3c2cnn3Cc2ccc(Cl)c(C(F)(F)F)c2)c1]

### Step 1: 4-chloro-1-(4-chloro-3-(trifluoromethyl)benzyl)-1H-pyrazolo[3,4-d]pyrimidin-6-amine

4-chloro-1-(4-chloro-3-(trifluoromethyl)benzyl)-1H-pyrazolo[3,4-d]pyrimidin-6-amine (201 mg, 31.37% yield) was obtained as an off-white solid in the same manner as in Example 6, except that Intermediate S1 (300 mg, 2.96 mmol) and 4-(bromomethyl)-1-chloro-2-(trifluoromethyl)benzene (533 mg, 1.95 mmol) were used, and Cs₂CO₃ was used instead of K₂CO₃. LCMS: m/z=362 (M+1, ESI+).

### Step 2: 5-(6-amino-1-(4-chloro-3-(trifluoromethyl)benzyl)-1H-pyrazolo[3,4-d]pyrimidin-4-yl)isophthalonitrile

To a stirred solution of 4-chloro-1-(4-chloro-3-(trifluoromethyl)benzyl)-1H-pyrazolo[3,4-d]pyrimidin-6-amine (185 mg, 0.51 mmol) and 5-(4,4,5,5-tetramethyl-1,3,2-dioxaborolan-2-yl)isophthalonitrile (168 mg, 0.66 mmol) in a mixture of THF (15 mL) and water (5 mL) was added K₂CO₃ (140 mg, 1.02 mmol) at 25° C., and the mixture was degassed with argon for 10 minutes. Thereafter, Pd(PPh₃)₄ (29.45 mg, 0.025 mmol) was added, and the mixture was stirred at 110° C. for 12 hours. After the reaction was completed, the reaction mixture was quenched with ice and extracted with EA (100 mL×2). The combined organic layer was washed with brine (15 mL), dried over Na₂SO₄, and concentrated. The residue was purified by column chromatography to afford the compound of Example 41 (98 mg, 42.24% yield) as an off-white solid. ¹H NMR (DMSO-d6) 8.81 (s, 2H), 8.70 (s, 1H), 8.61 (s, 1H), 7.80 (s, 1H), 7.70 (d, 1H), 7.44 (d, 1H), 7.23 (s, 2H), 5.56 (s, 2H); LCMS: m/z=454 (M+1, ESI+); MR: 228° C.-234° C.

### Example 42: 5-(6-amino-1-(2-chloro-3-(trifluoromethyl)benzyl)-1H-pyrazolo[3,4-d]pyrimidin-4-yl)isophthalonitrile

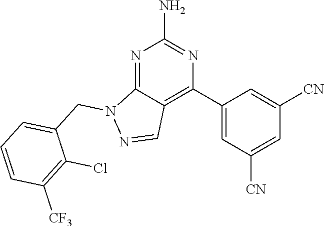 [SMILES: N#Cc1cc(C#N)cc(-c2nc(N)nc3c2cnn3Cc2cccc(C(F)(F)F)c2Cl)c1]

The compound of Example 42 (60 mg, 15.71% yield) was obtained as an off-white solid in the same manner as in Example 6 using Intermediate S2 (500 mg, crude, 0.842 mmol) and 1-(bromomethyl)-2-chloro-3-(trifluoromethyl)benzene (228 mg, 0.842 mmol). ¹H NMR (400 MHz, DMSO-d₆) δ 8.82 (d, 2H), 8.71 (s, 1H), 8.64 (s, 1H), 7.84 (d, 1H), 7.50 (t, 1H), 7.24 (s, 2H), 7.15 (d, 1H), 5.64 (s, 2H); LCMS: m/z=454 (M+1, ESI+); MR: 220° C.-226° C.

### Example 43: 5-(6-amino-1-(3-ethynyl-5-(trifluoromethyl)benzyl)-1H-pyrazolo[3,4-d]pyrimidin-4-yl)isophthalonitrile

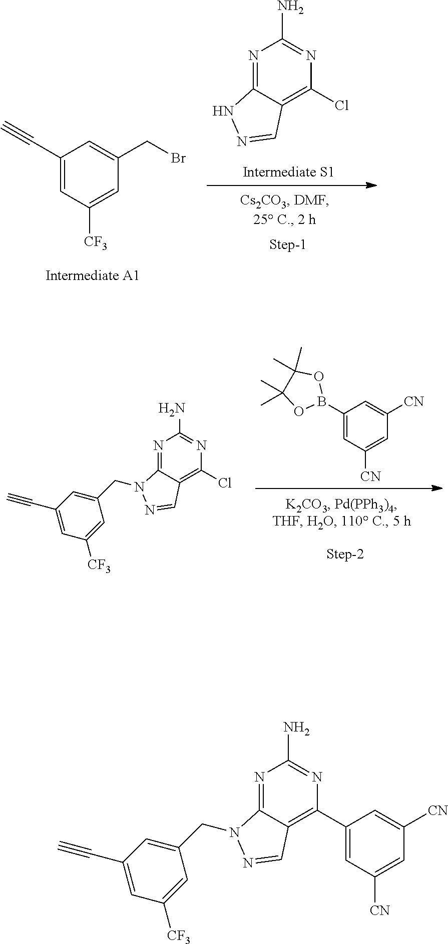 [SMILES: C#Cc1cc(CBr)cc(C(F)(F)F)c1 ; Nc1nc(Cl)c2cn[nH]c2n1 ; C#Cc1cc(Cn2ncc3c(Cl)nc(N)nc32)cc(C(F)(F)F)c1 ; CC1(C)OB(c2cc(C#N)cc(C#N)c2)OC1(C)C ; C#Cc1cc(Cn2ncc3c(-c4cc(C#N)cc(C#N)c4)nc(N)nc32)cc(C(F)(F)F)c1]

The compound of Example 43 (90 mg, 35.85% yield) was obtained as an off-white solid in the same manner as in Example 41 using Intermediate S1 (500 mg, 2.96 mmol) and Intermediate A1 (856 mg, 3.25 mmol). ¹H NMR (400 MHz, DMSO-d₆) δ 8.82 (s, 2H), 8.69 (s, 1H), 8.60 (s, 1H), 7.77 (s, 1H), 7.67 (s, 1H), 7.53 (s, 1H), 7.24 (s, 2H), 5.57 (s, 2H), 4.43 (s, 1H); LCMS: m/z=444 (M+1, ESI+); MR: 176° C.-182° C.

### Example 44: 5-(6-amino-1-(2,5-bis(trifluoromethyl)benzyl)-1H-pyrazolo[3,4-d]pyrimidin-4-yl)isophthalonitrile

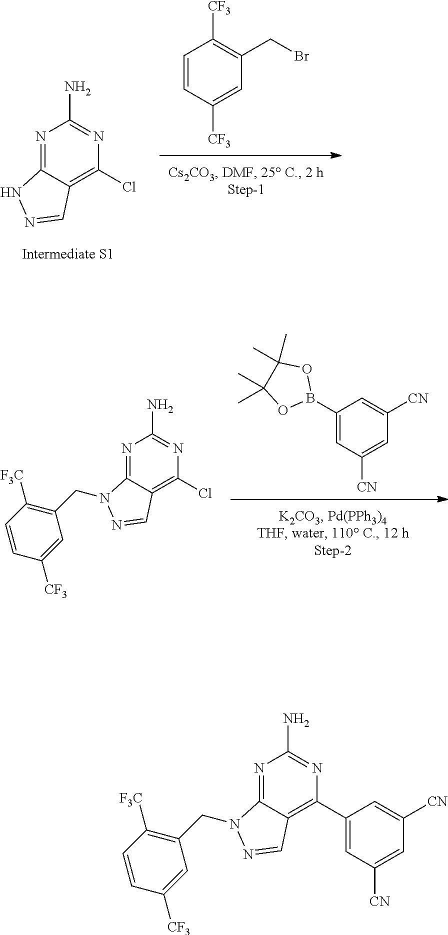 [SMILES: Nc1nc(Cl)c2cn[nH]c2n1 ; FC(F)(F)c1ccc(C(F)(F)F)c(CBr)c1 ; Nc1nc(Cl)c2cnn(Cc3cc(C(F)(F)F)ccc3C(F)(F)F)c2n1 ; CC1(C)OB(c2cc(C#N)cc(C#N)c2)OC1(C)C ; N#Cc1cc(C#N)cc(-c2nc(N)nc3c2cnn3Cc2cc(C(F)(F)F)ccc2C(F)(F)F)c1]

The compound of Example 44 (107 mg, 54.25% yield) was obtained as an off-white solid in the same manner as in Example 41 using Intermediate S1 (300 mg, 1.77 mmol) and 2-(bromomethyl)-1,4-bis(trifluoromethyl)benzene (543 mg, 1.77 mmol). ¹H NMR (400 MHz, DMSO-d₆) δ 8.84 (d, 2H), 8.70 (s, 1H), 8.66 (s, 1H), 8.08 (d, 1H), 7.96 (d, 1H), 7.33 (s, 1H), 7.26 (s, 2H), 5.72 (s, 2H); LCMS: m/z=488 (M+1, ESI+); MR: 244° C.-250° C.

### Example 45: 5-(6-amino-1-(2-methoxy-5-(trifluoromethyl)benzyl)-1H-pyrazolo[3,4-d]pyrimidin-4-yl)isophthalonitrile

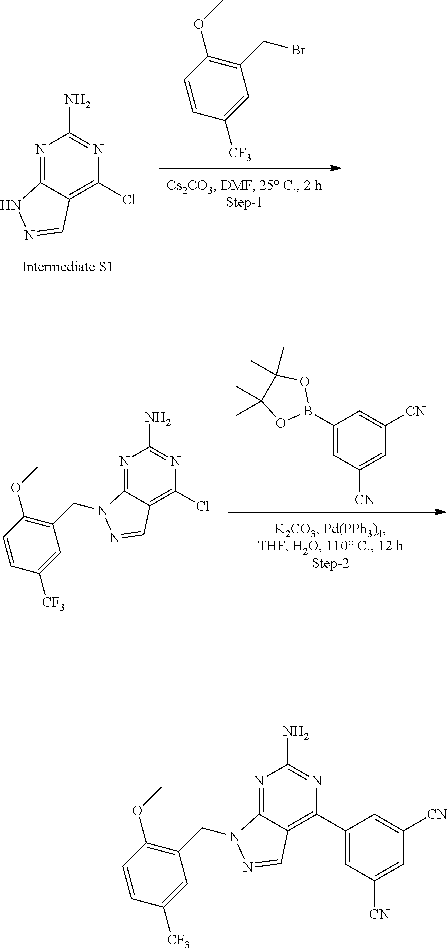 [SMILES: Nc1nc(Cl)c2cn[nH]c2n1 ; COc1ccc(C(F)(F)F)cc1CBr ; COc1ccc(C(F)(F)F)cc1Cn1ncc2c(Cl)nc(N)nc21 ; CC1(C)OB(c2cc(C#N)cc(C#N)c2)OC1(C)C ; COc1ccc(C(F)(F)F)cc1Cn1ncc2c(-c3cc(C#N)cc(C#N)c3)nc(N)nc21]

The compound of Example 45 (85 mg, 33.86% yield) was obtained as a pale yellow solid in the same manner as in Example 41 using Intermediate S1 (430 mg, 2.54 mmol) and 2-(bromomethyl)-1-methoxy-4-(trifluoromethyl)benzene (684 mg, 2.54 mmol). ¹H NMR (400 MHz, DMSO-d₆) δ 8.82 (d, 2H), 8.68 (s, 1H), 8.59 (s, 1H), 7.66 (d, 1H), 7.24 (d, 1H), 7.19 (s, 2H), 7.07 (s, 1H), 5.46 (s, 2H), 3.90 (s, 3H); LCMS: m/z=450 (M+1, ESI+); MR: 248° C.-254° C.

### Example 46: 5-(6-amino-1-(4-methoxy-3-(trifluoromethyl)benzyl)-1H-pyrazolo[3,4-d]pyrimidin-4-yl)isophthalonitrile

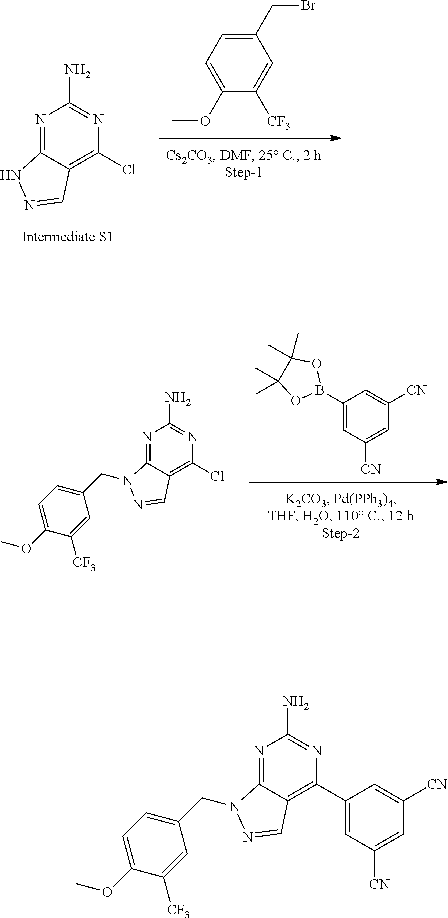 [SMILES: Nc1nc(Cl)c2cn[nH]c2n1 ; COc1ccc(CBr)cc1C(F)(F)F ; COc1ccc(Cn2ncc3c(Cl)nc(N)nc32)cc1C(F)(F)F ; CC1(C)OB(c2cc(C#N)cc(C#N)c2)OC1(C)C ; COc1ccc(Cn2ncc3c(-c4cc(C#N)cc(C#N)c4)nc(N)nc32)cc1C(F)(F)F]

The compound of Example 46 (70 mg, 26.7700 yield) was obtained as a pale yellow solid in the same manner as in Example 41 using Intermediate S1 (430 mg, 2.54 mmol) and 4-(bromomethyl)-1-methoxy-2-(trifluoromethyl)benzene (684 mg, 2.54 mmol). ¹H NMR (400 MHz, DMSO-d6) δ 8.81 (s, 2H), 8.69 (s, 1H), 8.56 (s, 1H), 7.56 (s, 1H), 7.49 (d, 1H), 7.24-7.21 (m, 3H), 5.47 (s, 2H), 3.86 (s, 3H); LCMS: m/z=450 (M+1, ESI+); MR: 202° C.-212° C.

### Example 47: 5-(6-amino-1-(2-methoxy-3-(trifluoromethyl)benzyl)-1H-pyrazolo[3,4-d]pyrimidin-4-yl)isophthalonitrile

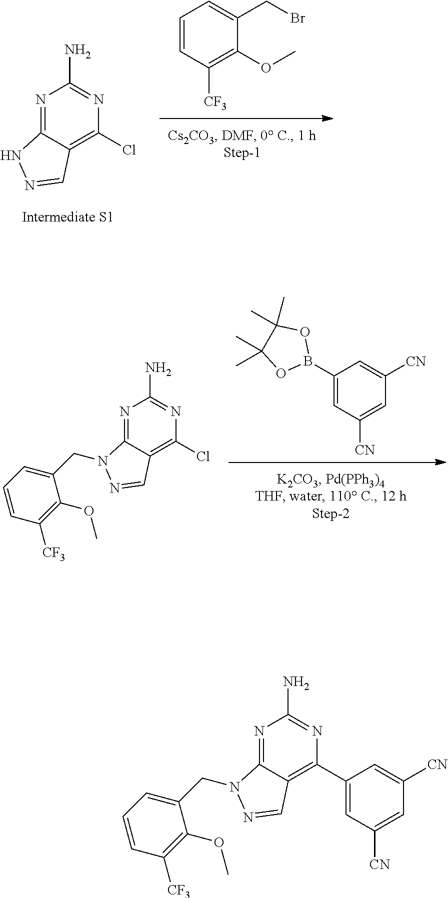 [SMILES: Nc1nc(Cl)c2cn[nH]c2n1 ; COc1c(CBr)cccc1C(F)(F)F ; COc1c(Cn2ncc3c(Cl)nc(N)nc32)cccc1C(F)(F)F ; CC1(C)OB(c2cc(C#N)cc(C#N)c2)OC1(C)C ; COc1c(Cn2ncc3c(-c4cc(C#N)cc(C#N)c4)nc(N)nc32)cccc1C(F)(F)F]

The compound of Example 47 (57 mg, 37.82% yield) was obtained as an off-white solid in the same manner as in Example 41 using Intermediate S1 (300 mg, 1.76 mmol) and 1-(bromomethyl)-2-methoxy-3-(trifluoromethyl)benzene (520 mg, 1.93 mmol). ¹H NMR (400 MHz, DMSO-d₆) δ 8.82 (d, 2H), 8.70 (s, 1H), 8.61 (s, 1H), 7.62 (d, 1H), 7.27-7.18 (m, 4H), 5.59 (s, 2H), 3.94 (s, 3H); LCMS: m/z=450 (M+1, ESI+); MR: 204° C.-212° C.

### Example 48: 5-(6-amino-1-(2-methyl-5-(trifluoromethyl)benzyl)-1H-pyrazolo[3,4-d]pyrimidin-4-yl)isophthalonitrile

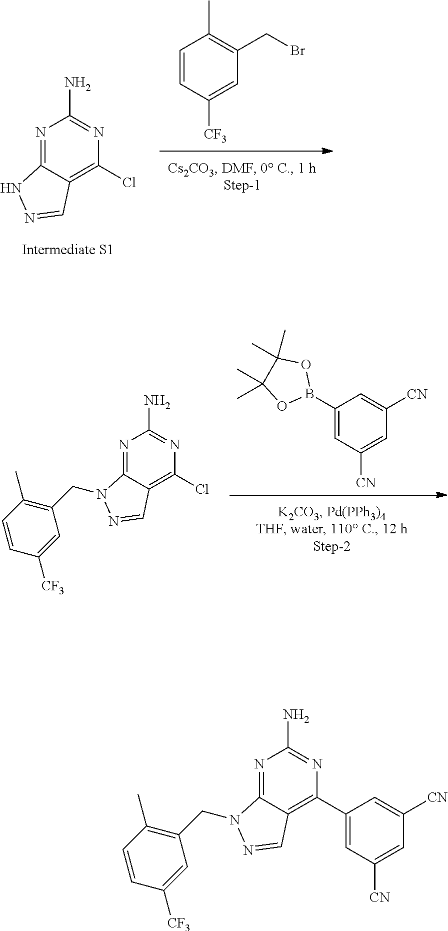 [SMILES: Nc1nc(Cl)c2cn[nH]c2n1 ; Cc1ccc(C(F)(F)F)cc1CBr ; Cc1ccc(C(F)(F)F)cc1Cn1ncc2c(Cl)nc(N)nc21 ; CC1(C)OB(c2cc(C#N)cc(C#N)c2)OC1(C)C ; Cc1ccc(C(F)(F)F)cc1Cn1ncc2c(-c3cc(C#N)cc(C#N)c3)nc(N)nc21]

The compound of Example 48 (121 mg, 47.71% yield) was obtained as an off-white solid in the same manner as in Example 41 using Intermediate S1 (250 mg, 1.47 mmol) and 2-(bromomethyl)-1-methyl-4-(trifluoromethyl)benzene (410 mg, 1.62 mmol). ¹H NMR (400 MHz, DMSO-d₆) δ 8.82 (d, 2H), 8.69 (s, 1H), 8.59 (s, 1H), 7.57 (d, 1H), 7.46 (d, 1H), 7.22 (s, 3H), 5.53 (s, 2H), 2.43 (s, 3H); LCMS: m/z=434 (M+1, ESI+); MR: 204° C.-210° C.

### Example 49: 5-(6-amino-1-(4-methyl-3-(trifluoromethyl)benzyl)-1H-pyrazolo[3,4-d]pyrimidin-4-yl)isophthalonitrile

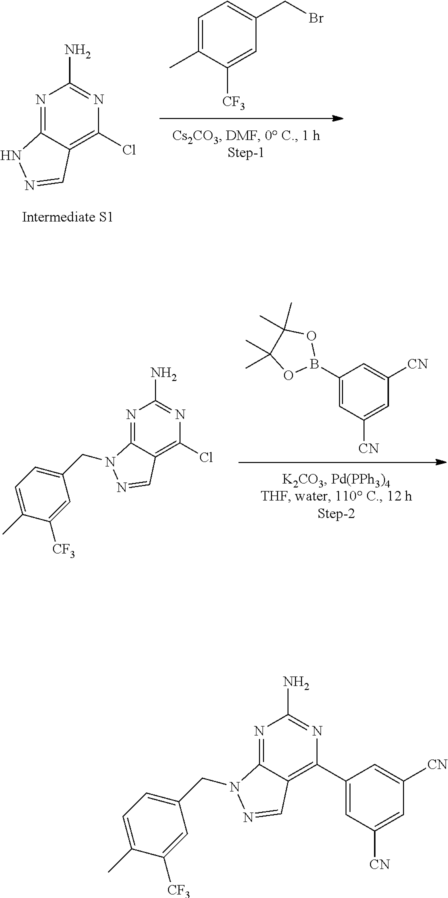 [SMILES: Nc1nc(Cl)c2cn[nH]c2n1 ; Cc1ccc(CBr)cc1C(F)(F)F ; Cc1ccc(Cn2ncc3c(Cl)nc(N)nc32)cc1C(F)(F)F ; CC1(C)OB(c2cc(C#N)cc(C#N)c2)OC1(C)C ; Cc1ccc(Cn2ncc3c(-c4cc(C#N)cc(C#N)c4)nc(N)nc32)cc1C(F)(F)F]

The compound of Example 49 (60 mg, 23.65% yield) was obtained as an off-white solid in the same manner as in Example 41 using Intermediate S1 (300 mg, 1.76 mmol) and 4-(bromomethyl)-1-methyl-2-(trifluoromethyl)benzene (489 mg, 1.93 mmol). ¹H NMR (400 MHz, DMSO-d₆) δ 8.81 (d, 2H), 8.69 (t, 1H), 8.58 (s, 1H), 7.59 (s, 1H), 7.41-7.34 (m, 2H), 7.21 (s, 2H), 5.51 (s, 2H), 2.40 (s, 3H); LCMS: m/z=434 (M+1, ESI+); MR: 176° C.-182° C.

### Example 50: 5-(6-amino-1-(2-methyl-3-(trifluoromethyl)benzyl)-1H-pyrazolo[3,4-d]pyrimidin-4-yl)isophthalonitrile

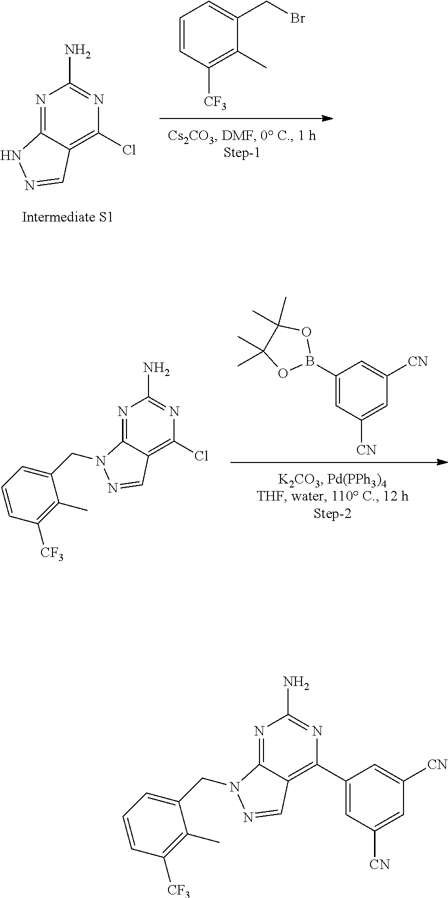 [SMILES: Nc1nc(Cl)c2cn[nH]c2n1 ; Cc1c(CBr)cccc1C(F)(F)F ; Cc1c(Cn2ncc3c(Cl)nc(N)nc32)cccc1C(F)(F)F ; CC1(C)OB(c2cc(C#N)cc(C#N)c2)OC1(C)C ; Cc1c(Cn2ncc3c(-c4cc(C#N)cc(C#N)c4)nc(N)nc32)cccc1C(F)(F)F]

The compound of Example 50 (110 mg, 43.37% yield) was obtained as an off-white solid in the same manner as in Example 41 using Intermediate S1 (300 mg, 1.76 mmol) and 4-(bromomethyl)-1-methyl-2-(trifluoromethyl)benzene (445 mg, 1.76 mmol). ¹H NMR (400 MHz, DMSO-d₆) δ 8.81 (d, 2H), 8.69 (s, 1H), 8.59 (s, 1H), 7.62 (d, 1H), 7.31 (t, 1H), 7.19 (s, 2H), 7.05 (d, 1H), 5.56 (s, 2H), 2.50-2.48 (m, 3H); LCMS: m/z=434 (M+1, ESI+).

### Example 51: 5-(6-amino-1-(2-fluoro-5-(trifluoromethyl)benzyl)-1H-pyrazolo[3,4-d]pyrimidin-4-yl)isophthalonitrile

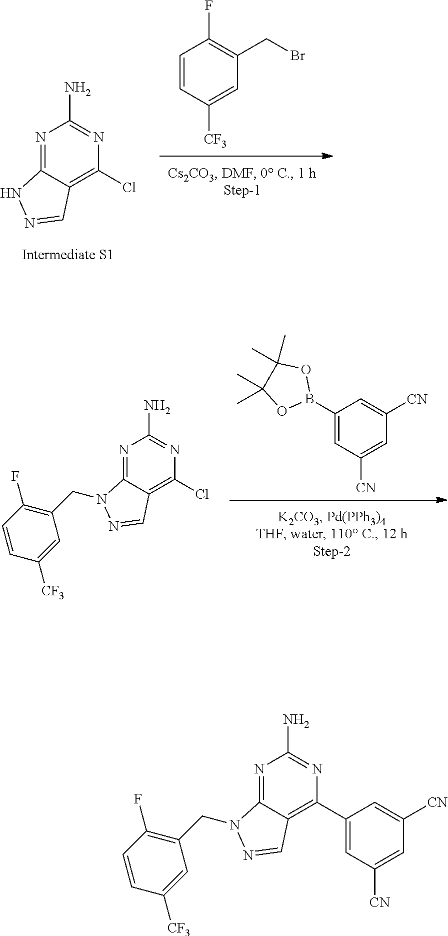 [SMILES: Nc1nc(Cl)c2cn[nH]c2n1 ; Fc1ccc(C(F)(F)F)cc1CBr ; Nc1nc(Cl)c2cnn(Cc3cc(C(F)(F)F)ccc3F)c2n1 ; CC1(C)OB(c2cc(C#N)cc(C#N)c2)OC1(C)C ; N#Cc1cc(C#N)cc(-c2nc(N)nc3c2cnn3Cc2cc(C(F)(F)F)ccc2F)c1]

The compound of Example 51 (110 mg, 57.95% yield) was obtained as an off-white solid in the same manner as in Example 41 using Intermediate S1 (300 mg, 1.76 mmol) and 2-(bromomethyl)-1-fluoro-4-(trifluoromethyl)benzene (497 mg, 1.93 mmol). ¹H NMR (400 MHz, DMSO-d₆) δ 8.81 (d, 2H), 8.69 (d, 1H), 8.57 (s, 1H), 7.80-7.79 (m, 1H), 7.63 (d, 1H), 7.50 (t, 1H), 7.24 (s, 2H), 5.58 (s, 2H); LCMS: m/z=438 (M+1, ESI+); MR: 206° C.-212° C.

### Example 52: 5-(6-amino-1-(4-fluoro-3-(trifluoromethyl)benzyl)-1H-pyrazolo[3,4-d]pyrimidin-4-yl)isophthalonitrile

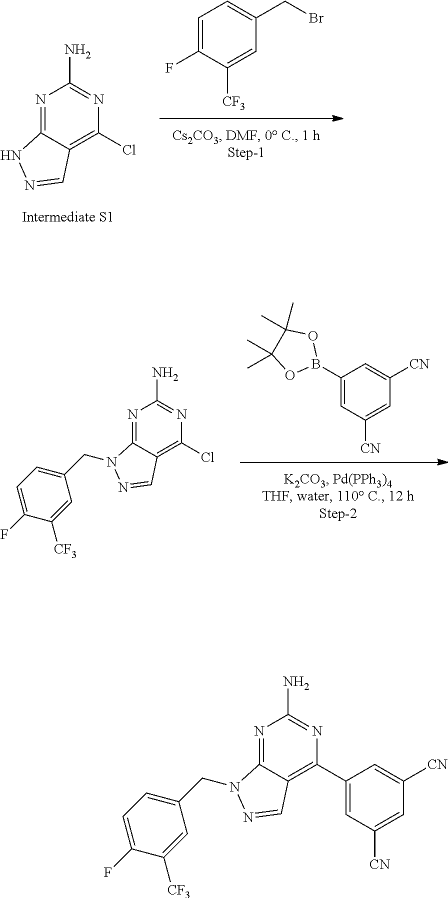 [SMILES: Nc1nc(Cl)c2cn[nH]c2n1 ; Fc1ccc(CBr)cc1C(F)(F)F ; Nc1nc(Cl)c2cnn(Cc3ccc(F)c(C(F)(F)F)c3)c2n1 ; CC1(C)OB(c2cc(C#N)cc(C#N)c2)OC1(C)C ; N#Cc1cc(C#N)cc(-c2nc(N)nc3c2cnn3Cc2ccc(F)c(C(F)(F)F)c2)c1]

The compound of Example 52 (98 mg, 38.68% yield) was obtained as an off-white solid in the same manner as in Example 41 using Intermediate S1 (300 mg, 1.76 mmol) and 4-(bromomethyl)-1-fluoro-2-(trifluoromethyl)benzene (496 mg, 1.93 mmol). ¹H NMR (400 MHz, DMSO-d₆) δ 8.80 (s, 2H), 8.68 (s, 1H), 8.58 (s, 1H), 7.71 (d, 1H), 7.53-7.45 (m, 2H), 7.22 (s, 2H), 5.54 (s, 2H); LCMS: m/z=438 (M+1, ESI+).

### Example 53: 5-(6-amino-1-(2-fluoro-3-(trifluoromethyl)benzyl)-1H-pyrazolo[3,4-d]pyrimidin-4-yl)isophthalonitrile

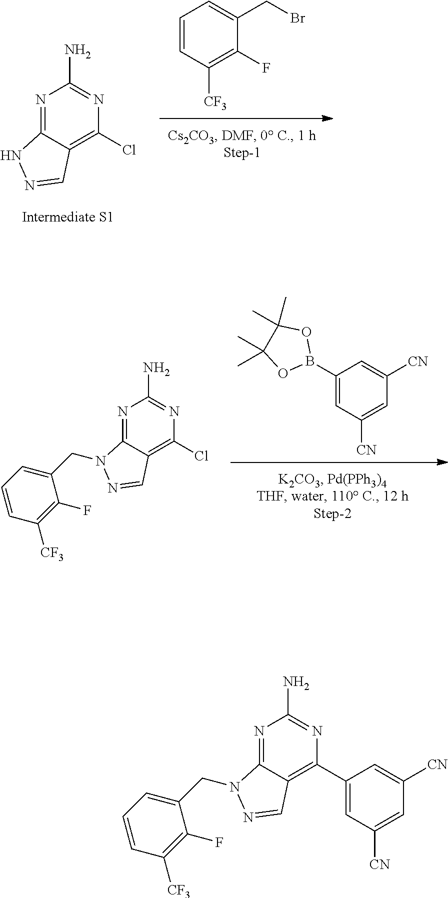 [SMILES: Nc1nc(Cl)c2cn[nH]c2n1 ; Fc1c(CBr)cccc1C(F)(F)F ; Nc1nc(Cl)c2cnn(Cc3cccc(C(F)(F)F)c3F)c2n1 ; CC1(C)OB(c2cc(C#N)cc(C#N)c2)OC1(C)C ; N#Cc1cc(C#N)cc(-c2nc(N)nc3c2cnn3Cc2cccc(C(F)(F)F)c2F)c1]

The compound of Example 53 (119 mg, 46.94% yield) was obtained as an off-white solid in the same manner as in Example 41 using Intermediate S1 (300 mg, 1.76 mmol) and 1-(bromomethyl)-2-fluoro-3-(trifluoromethyl)benzene (497 mg, 1.93 mmol). ¹H NMR (400 MHz, DMSO-d₆) δ 8.80 (d, 2H), 8.69 (d, 1H), 8.58 (s, 1H), 7.75 (t, 1H), 7.47 (t, 1H), 7.38 (t, 1H), 7.23 (s, 2H), 5.59 (s, 2H); LCMS: m/z=438 (M+1, ESI+); MR: 188° C.-194° C.

### Example 54: 5-(5-amino-3-(2-fluoro-3-(trifluoromethyl)benzyl)-3H-[1,2,3]triazolo[4,5-d]pyrimidin-7-yl)isophthalonitrile

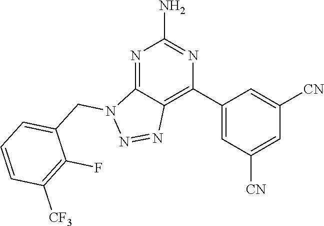 [SMILES: N#Cc1cc(C#N)cc(-c2nc(N)nc3c2nnn3Cc2cccc(C(F)(F)F)c2F)c1]

The compound of Example 54 (110 mg, 6.26% yield) was obtained as an off-white solid using in the same manner as in Example 20, except that 1-(bromomethyl)-2-fluoro-3-(trifluoromethyl)benzene was used instead of 1-(bromomethyl)-3-(trifluoromethyl)benzene in Example 20. ¹H NMR (400 MHz, DMSO-d6) δ 9.25 (d, 2H), 8.77 (t, 1H), 7.80 (t, 1H), 7.64-7.62 (m, 3H), 7.42 (t, 1H), 5.86 (s, 2H); LCMS: m/z=439 (M+1, ESI+); MR: 244° C.-248° C.

### Example 55: 5-(2-amino-9-(2-fluoro-3-(trifluoromethyl)benzyl)-9H-purin-6-yl)isophthalonitrile

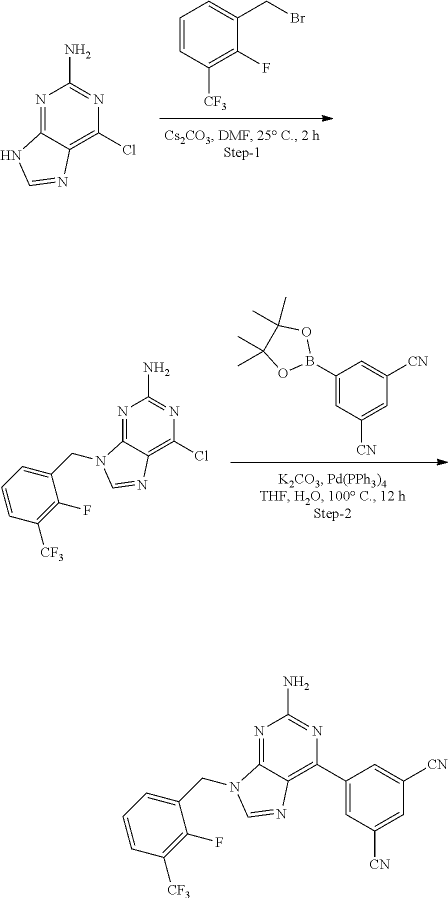 [SMILES: Nc1nc(Cl)c2nc[nH]c2n1 ; Fc1c(CBr)cccc1C(F)(F)F ; Nc1nc(Cl)c2ncn(Cc3cccc(C(F)(F)F)c3F)c2n1 ; CC1(C)OB(c2cc(C#N)cc(C#N)c2)OC1(C)C ; N#Cc1cc(C#N)cc(-c2nc(N)nc3c2ncn3Cc2cccc(C(F)(F)F)c2F)c1]

The compound of Example 55 (290 mg, 57.30% yield) was obtained as an off-white solid in the same manner as in Example 41 using 6-chloro-9H-purin-2-amine (0.4 g, 2.36 mmol) and 1-(bromomethyl)-2-fluoro-3-(trifluoromethyl)benzene (0.6 g, 2.36 mmol). ¹H NMR (400 MHz, DMSO-d6) δ 9.31 (s, 2H), 8.65-8.64 (m, 1H), 8.39 (s, 1H), 7.76 (t, 1H), 7.46 (t, 1H), 7.38 (t, 1H), 6.84 (brs, 2H), 5.52 (s, 2H); LCMS: m/z=438 (M+1, ESI+); MR: 247° C.-251° C.

### Example 56: 5-(2-amino-9-(2-fluoro-3-(trifluoromethyl)benzyl)-8-methyl-9H-purin-6-yl)isophthalonitrile

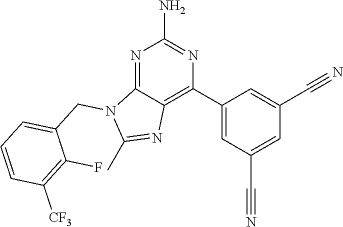 [SMILES: Cc1nc2c(-c3cc(C#N)cc(C#N)c3)nc(N)nc2n1Cc1cccc(C(F)(F)F)c1F]

Intermediate S9 (210 mg, 0.76 mmol) was dissolved in DMF (8 mL) solution, and then Cs₂CO₃ (494 mg, 1.52 mmol) was added at 0° C. After 10 minutes, 1-(bromomethyl)-2-fluoro-3-(trifluoromethyl)benzene (214 mg, 0.83 mmol) was added at 0° C. The mixture was stirred at 0° C. for 2 hours and quenched with ice to terminate the reaction. The precipitate formed from the ice-cooled reaction mixture was filtered and washed with water (30 mL). Thereafter, the residue was purified by column chromatography to afford the compound of Example 56 (72 mg, 20.93% yield) as an off-white solid. ¹H NMR (400 MHz, DMSO-d6) δ 9.31 (d, 2H), 8.63 (t, 1H), 7.75 (t, 1H), 7.38-7.28 (m, 2H), 6.73 (s, 2H), 5.49 (s, 2H), 2.53 (s, 3H); LCMS: m/z=452 (M+1, ESI+); MR: not melt up to 300° C.

### Example 57: 5-(6-amino-1-(2-fluoro-3-(trifluoromethyl)benzyl)-3-methyl-11H-pyrazolo[3,4-d]pyrimidin-4-yl)isophthalonitrile

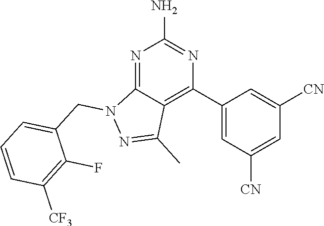

To a stirred solution of Intermediate S10 (0.15 g, 0.54 mmol) in DMF (10 mL) was added Cs₂CO₃ (0.26 g, 0.81 mmol) at 0° C. After 10 minutes, 1-(bromomethyl)-2-fluoro-3-(trifluoromethyl)benzene (0.14 g, 0.54 mmol) was added, and the mixture was stirred at 25° C. for 2 hours. After the reaction was completed, the reaction mixture was quenched with ice water (30 mL) and extracted with EA (2×50 mL). The organic phase was washed with ice water (20 mL×2) and brine (20 mL), dried over Na₂SO₄, and concentrated to afford a residue. The residue was purified by column chromatography to afford the compound of Example 57 (0.15 g, 60.98% yield) as an off-white solid. ¹H NMR (400 MHz, DMSO-d6) δ 8.68 (s, 1H), 8.53 (s, 2H), 7.50 (t, 1H), 7.49 (t, 1H), 7.39 (t, 1H), 7.16 (brs, 2H), 5.50 (s, 2H), 2.13 (s, 3H); LCMS: m/z=452 (M+1, ESI+); MR:190° C.-196° C.

### Example 58: 3-(6-amino-1-(2-fluoro-3-(trifluoromethyl)benzyl)-1H-pyrazolo[3,4-d]pyrimidin-4-yl)-5-cyanobenzamide

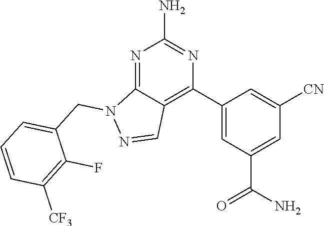 [SMILES: N#Cc1cc(C(N)=O)cc(-c2nc(N)nc3c2cnn3Cc2cccc(C(F)(F)F)c2F)c1]

The compound of Example 53 (230 mg, 0.526 mmol) was added to toluene (10 mL). Indium(III) chloride (5.82 mg, 0.026 mmol) and (Z)-acetaldehyde oxime (0.224 mL, 3.15 mmol) were added, and the mixture was stirred at 70° C. for 20 minutes. After the reaction was completed, the solvent was evaporated, and the residue was diluted with water (30 mL) and then extracted with EA (100 mL×2). The combined organic layer was dried over Na₂SO₄ and concentrated, and then the residue was purified by column chromatography to afford the compound of Example 58 (96 mg, 40.16% yield) as an off-white solid. ¹H NMR (400 MHz, DMSO-d₆) δ 8.83 (s, 1H), 8.63 (s, 1H), 8.47 (d, 2H), 8.33 (s, 1H), 7.78-7.73 (m, 2H), 7.47 (t, 1H), 7.38 (t, 1H), 7.20 (s, 2H), 5.59 (s, 2H); LCMS: m/z=456 (M+1, ESI+); M.R: 258° C.-261° C.

### Example 59: 3-(2-amino-9-(2-fluoro-3-(trifluoromethyl)benzyl)-9H-purin-6-yl)-5-cyanobenzamide

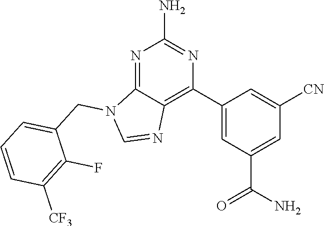 [SMILES: N#Cc1cc(C(N)=O)cc(-c2nc(N)nc3c2ncn3Cc2cccc(C(F)(F)F)c2F)c1]

The compound of Example 59 (46 mg, 21.04% yield) was obtained as an off-white solid in the same manner as in Example 58 using the compound of Example 55 (0.21 g, 0.48 mmol) as a starting material.

¹H NMR (400 MHz, DMSO-d6) δ 9.44 (s, 1H), 9.26 (s, 1H), 8.45 (s, 1H), 8.37 (s, 1H), 8.27 (s, 1H), 7.79-7.77 (m, 2H), 7.46-7.38 (m, 2H), 6.80 (s, 2H), 5.53 (s, 2H); LCMS: m/z=456 (M+1, ESI+); MR: 244° C.-250° C.

### Example 60: 3-(5-amino-3-(2-fluoro-3-(trifluoromethyl)benzyl)-3H-[1,2,3]triazolo[4,5-d]pyrimidin-7-yl)-5-cyanobenzamide

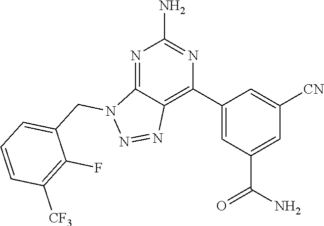 [SMILES: N#Cc1cc(C(N)=O)cc(-c2nc(N)nc3c2nnn3Cc2cccc(C(F)(F)F)c2F)c1]

The compound of Example 60 (32 mg, 30.76% yield) was obtained as an off-white solid in the same manner as in Example 58 using the compound of Example 54 (100 mg, 0.22 mmol) as a starting material. ¹H NMR (400 MHz, DMSO-d6) δ 9.44 (s, 1H), 9.18 (s, 1H), 8.56 (s, 1H), 8.31 (s, 1H), 7.81-7.78 (m, 2H), 7.64-7.60 (m, 3H), 7.42 (t, 1H), 5.85 (s, 2H); LCMS: m/z=457 (M+1, ESI+); MR: not melt up to 300° C.

### Example 61: 5-(6-amino-1-(5-chloro-2-fluoro-3-(trifluoromethyl)benzyl)-1H-pyrazolo[3,4-d]pyrimidin-4-yl)isophthalonitrile

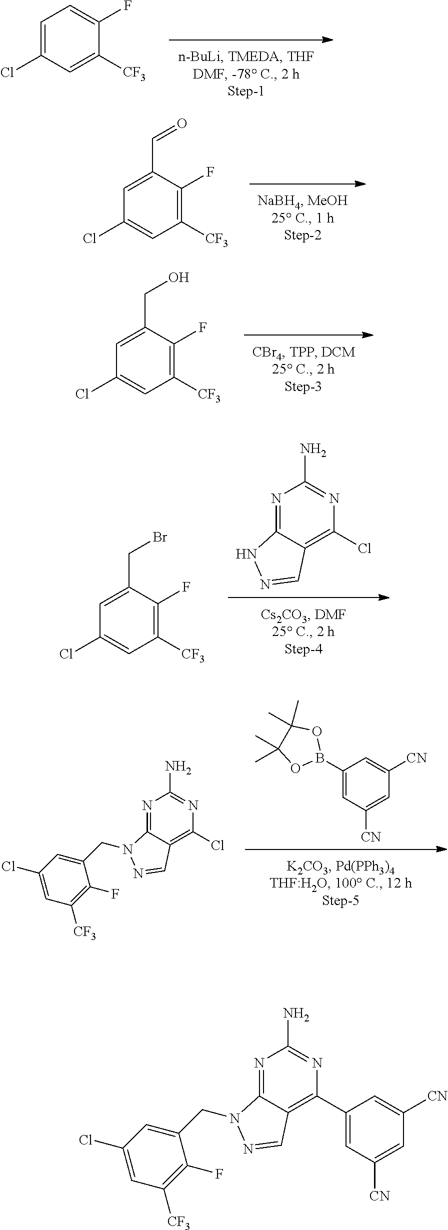

### Step 1: 5-chloro-2-fluoro-3-(trifluoromethyl)benzaldehyde

4-chloro-1-fluoro-2-(trifluoromethyl)benzene (0.5 g, 2.52 mmol) was dissolved in THF (30 mL) and then stirred for 10 minutes at room temperature. n-BuLi (1.89 mL, 3.0 mmol) was added to the reaction mixture at −78° C. and stirred at the same temperature for 1 hour. DMF (0.24 mL, 3.0 mmol) was added to the reaction mixture and stirred at 25° C. for an additional hour. After the reaction was completed, the reaction mixture was quenched with aqueous ammonium chloride and extracted with EA (50 mL×2). The combined organic layer was washed with water (10 mL×2) and brine (10 mL), dried over Na₂SO₄, and concentrated to afford 5-chloro-2-fluoro-3-(trifluoromethyl)benzaldehyde (0.5 g, crude) as a sticky black liquid. ¹H NMR (400 MHz, DMSO-d₆) δ 10.15 (s, 1H), 8.25 (dd, 1H), 8.16 (dd, 1H).

### Step 2: (5-chloro-2-fluoro-3-(trifluoromethyl)phenyl)methanol

To a solution of 5-chloro-2-fluoro-3-(trifluoromethyl)benzaldehyde (2 g, crude) at 0° C. was added MeOH (30 mL). NaBH₄ (0.33 g, 8.8 mmol) was added, and the mixture was stirred at 25° C. for 1 hour. After the reaction was completed, the solvent was concentrated, and the residue was diluted with ice water (20 mL) and then extracted with DCM (50 mL×2). The combined organic layer was washed with water (10 mL) and brine (10 mL), dried over Na₂SO₄, and concentrated, and then the residue was purified by column chromatography to afford (5-chloro-2-fluoro-3-(trifluoromethyl)phenyl)methanol (1 g, quantitative) as a pale yellow solid. ¹H NMR (400 MHz, DMSO-d6) δ 7.80 (t, 2H), 5.60 (t, 1H), 4.61 (d, 2H).

### Step 3: 1-(bromomethyl)-5-chloro-2-fluoro-3-(trifluoromethyl)benzene

To a solution of 5-chloro-2-fluoro-3-(trifluoromethyl)phenyl)methanol (1 g, 4.37 mmol) at 0° C. was added DCM (30 mL). TPP (2.3 g, 8.7 mmol) and CBr₄ (2.9 g, 8.7 mmol) were added, and the mixture was stirred at 25° C. for 1 hour. Upon completion, the reaction mixture was cooled with ice and extracted with DCM (50 mL×2). The combined organic layer was washed with water (10 mL) and brine (20 mL), dried over Na₂SO₄, and concentrated, and then the residue was purified by column chromatography to afford 1-(bromomethyl)-5-chloro-2-fluoro-3-(trifluoromethyl)benzene (0.9 g, 70.86% yield) as an off-white solid. ¹H NMR (400 MHz, DMSO-d₆) δ 8.06 (dd, 1H), 7.91 (dd, 1H), 4.74 (s, 2H).

### Step 4: 4-chloro-1-(5-chloro-2-fluoro-3-(trifluoromethyl)benzyl)-1H-pyrazolo[3,4-d]pyrimidin-6-amine

To a solution of 4-chloro-1H-pyrazolo[3,4-d]pyrimidin-6-amine (lg, 5.9 mmol) at 0° C. was added DMF (20 mL). The mixture was stirred at the same temperature for 10 minutes, and then 1-(bromomethyl)-5-chloro-2-fluoro-3-(trifluoromethyl)benzene was added. The mixture was stirred at 25° C. for 2 hours. The reaction mixture was cooled with ice and extracted with EA (50 mL×2). Thereafter, the combined organic layer was washed with water (20 mL) and brine (20 mL), dried over Na₂SO₄, and concentrated. The residue was purified by column chromatography to afford 4-chloro-1-(5-chloro-2-fluoro-3-(trifluoromethyl)benzyl)-1H-pyrazolo[3,4-d]pyrimidin-6-amine (350 mg, 26.90% yield) as a pale yellow solid. LCMS: m/z=380 (M+1, ESI+).

### Step 5: 5-(6-amino-1-(5-chloro-2-fluoro-3-(trifluoromethyl)benzyl)-1H-pyrazolo[3,4-d]pyrimidin-4-yl)isophthalonitrile

4-chloro-1-(5-chloro-2-fluoro-3-(trifluoromethyl)benzyl)-1H-pyrazolo[3,4-d]pyrimidin-6-amine (350 mg, 0.9 mmol) and 5-(4,4,5,5-tetramethyl-1,3,2-dioxaborolan-2-yl)isophthalonitrile (297 mg, 1.17 mmol) were added to a mixture of THF (15 mL) and water (5 mL), and K₂CO₃ (254 mg, 1.8 mmol) was added at 25° C. After filling with argon for 10 minutes, the mixture was stirred. Pd(Ph₃)₄ (53 mg, 0.01 mmol) was added, and the mixture was stirred at 100° C. for 12 hours. After the reaction was completed, the reaction mixture was filtered through a Celite bed and washed with EA (600 mL) and MeOH (200 mL). The filtrate was concentrated, and the residue was purified by column chromatography to afford the compound of Example 61 (80 mg, 18.43% yield) as an off-white solid. ¹H NMR (400 MHz, DMSO-d6) δ 8.80 (s, 2H), 8.69 (s, 1H), 8.58 (s, 1H), 7.89 (m, 1H), 7.62-7.61 (m, 1H), 7.25 (s, 2H), 5.57 (s, 2H); LCMS: m/z=472 (M+1, ESI+); MR: 230° C.-236° C.

### Example 62: 3-(6-amino-1-(5-chloro-2-fluoro-3-(trifluoromethyl)benzyl)-1H-pyrazolo[3,4-d]pyrimidin-4-yl)-5-cyanobenzamide

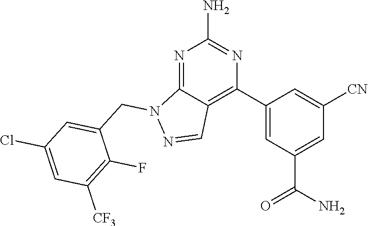 [SMILES: N#Cc1cc(C(N)=O)cc(-c2nc(N)nc3c2cnn3Cc2cc(Cl)cc(C(F)(F)F)c2F)c1]

The compound of Example 62 (9 mg, 21.95% yield) was obtained as an off-white solid in the same manner as in Example 58 using the compound of Example 61 (40 mg, 0.08 mmol) as a starting material. ¹H NMR (400 MHz, DMSO-d6) δ 8.84 (s, 1H), 8.64 (s, 1H), 8.48-8.47 (m, 2H), 8.33 (s, 1H), 7.91-7.89 (m, 1H), 7.79 (s, 1H), 7.64-7.62 (m, 1H), 7.23 (s, 2H), 5.58 (s, 2H); LCMS: m/z=490 (M+1, ESI+).

### Example 63: 5-(6-amino-1-(2,5-difluoro-3-(trifluoromethyl)benzyl)-1H-pyrazolo[3,4-d]pyrimidin-4-yl)isophthalonitrile

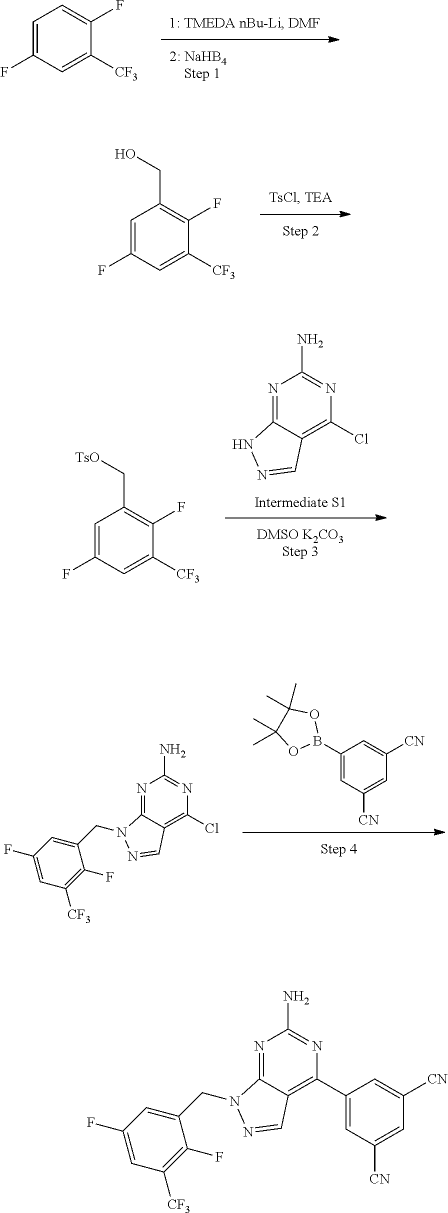

### Step 1: (2,5-difluoro-3-(trifluoromethyl)phenyl)methanol

1,4-difluoro-2-(trifluoromethyl)benzene (5.00 g, 27.31 mmol) and tetramethylethylene diamine (TMEDA, 4.76 g, 40.96 mmol) were dissolved in THF (50 mL), and At −78° C., n-BuLi (1.6 M in hexane) (22 mL, 35.50 mmol) was added dropwise, and the mixture was stirred at the same temperature for 90 minutes. DMF (2.59 g, 35.50 mmol) was added, and the mixture was stirred at the same temperature for 30 minutes. Thereafter, sodium borate (2.07 g, 54.62 mmol) was slowly added while maintaining the mixture at 0° C. in an ice bath. The resulting mixture was stirred at room temperature for 2 hours, and then the reaction mixture was diluted with water (500 mL) and extracted with EtOAc (500 mL×3). Thereafter, the organic layer was dried over anhydrous Na₂SO₄ (s) and concentrated. The resulting residue was purified by silica gel column chromatography (PE: EtOAc=5: 1) to afford (2,5-difluoro-3-(trifluoromethyl)phenyl)methanol (3.00 g, 51.55% yield) as a yellow oil. ¹H NMR (400 MHz, DMSO-d₆) δ: 7.66-7.51 (m, 2H), 5.67-5.62 (m, 2H), 4.63 (d, 2H).

### Step 2: 2,5-difluoro-3-(trifluoromethyl)benzyl 4-methylbenzenesulfonate

(2,5-difluoro-3-(trifluoromethyl)phenyl)methanol (3.00 g, 14.07 mmol) and tri ethylamine (2.85 g, 28.15 mmol) were dissolved in DCM (30 mL), and then TsCl (3.49 g, 18.30 mmol) was added at 0° C. The mixture was stirred at 0° C. for 1 hour, and the reaction mixture was diluted with water (100 mL) and then extracted with DCM (100 mL×3). The organic layer was dried over anhydrous Na₂SO₄ and concentrated to afford 2,5-difluoro-3-(trifluoromethyl)benzyl 4-methylbenzenesulfonate (6.0 g, 100% yield) as a white oil without further purification.

### Step 3: 4-chloro-1-(2,5-difluoro-3-(trifluoromethyl)benzyl)-1H-pyrazolo[3,4-d]pyrimidin-6-amine

Intermediate S1 (500 mg, 2.96 mmol), 2,5-difluoro-3-(trifluoromethyl)benzyl 4-methylbenzenesulfonate (1.3 g, 3.55 mmol), and K₂CO₃ (817 mg, 5.92 mmol) were added to DMSO (10 mL), and the mixture was stirred at 0° C. for 1 hour. The reaction mixture was diluted with water (20 mL) and extracted with EA (20 mL*2). The combined organic phase was washed with brine (30 mL), dried over Na₂SO₄, and concentrated and purified by silica gel column chromatography (PE: EtOAc=3: 1) to afford 4-chloro-1-(2,5-difluoro-3-(trifluoromethyl)benzyl)-1H-pyrazolo[3,4-d]pyrimidin-6-amine (100 mg, 9.34% yield) as a white solid. MS: m/z=364.0 (M+1, ESI+)

### Step 4: 5-(6-amino-1-(2,5-difluoro-3-(trifluoromethyl)benzyl)-1H-pyrazolo[3,4-d]pyrimidin-4-yl)isophthalonitrile

4-chloro-1-(2,5-difluoro-3-(trifluoromethyl)benzyl)-1H-pyrazolo[3,4-d]pyrimidin-6-amine (100 mg, 0. 27 mmol), 5-(4,4,5,5-tetramethyl-1,3,2-dioxaborolan-2-yl)isophthalonitrile (105 mg, 0.41 mmol), Pd(dppf)Cl₂ (22 mg, 0.03 mmol), and K₂CO₃ (74 mg, 0.54 mmol) were added to 1,4-dioxane:H₂O (4:1) (5 mL), and the mixture was stirred at 100° C. for 2 hours. The reaction mixture was concentrated, and the residue was purified by silica gel column chromatography (PE:EtOAc=1:1) to afford the compound of Example 63 (28 mg, 22.76% yield) as a white solid. ¹H NMR (400 MHz, DMSO-d₆) δ: 8.80 (s, 2H), 8.69 (s, 1H), 8.59 (s, 1H), 7.75-7.73 (m, 1H), 7.39-7.38 (m, 1H), 7.24 (s, 2H), 5.58 (s, 2H); MS: m/z=455.9 (M+1, ESI+).

### Example 64: 3-(6-amino-1-(2,5-difluoro-3-(trifluoromethyl)benzyl)-1H-pyrazolo[3,4-d]pyrimidin-4-yl)-5-cyanobenzamide

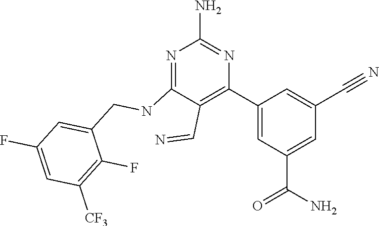 [SMILES: N#Cc1cc(C(N)=O)cc(-c2nc(N)nc(NCc3cc(F)cc(C(F)(F)F)c3F)c2C=N)c1]

The compound of Example 63 (150 mg, 0. 33 mmol) was dissolved in 1,4-dioxane (1 mL). Acetaldoxime (97 mg, 1.64 mmol) and InCl₃ (7 mg, 0.03 mmol) were added to toluene (1 mL), and then the mixture was stirred at 90° C. for 50 minutes. The mixture was concentrated to obtain a residue, which was purified by pre-HPLC (ACN/H₂O=40/60) to afford the compound of Example 64 (10 mg, 6.41% yield) as a white solid. ¹H NMR (400 MHz, DMSO-d₆) δ: 8.84-8.83 (m, 1H), 8.64-8.63 (m, 1H), 8.48-8.47 (m, 2H), 8.33 (s, 1H), 7.78-7.72 (m, 2H), 7.42-7.38 (m, 1H), 7.22 (s, 2H), 5.58 (s, 2H); MS: m/z=474.0 (M+1, ESI+).

### Example 65: 5-(2-amino-9-(2,5-difluoro-3-(trifluoromethyl)benzyl)-9H-purin-6-yl)isophthalonitrile

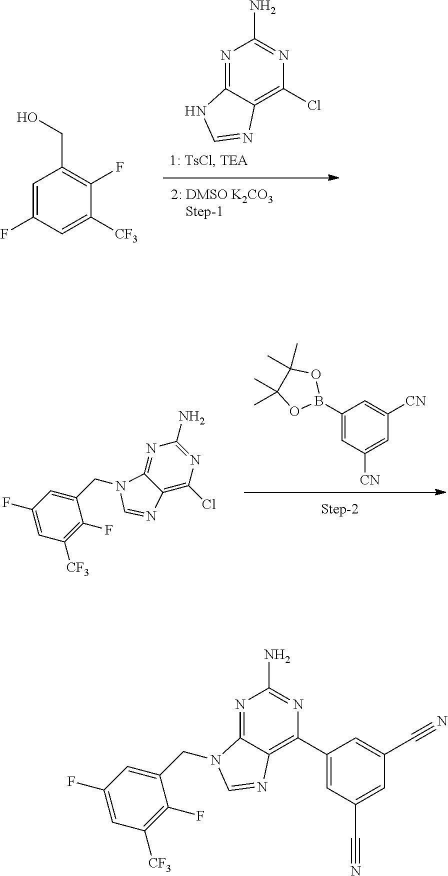 [SMILES: OCc1cc(F)cc(C(F)(F)F)c1F ; Nc1nc(Cl)c2nc[nH]c2n1 ; Nc1nc(Cl)c2ncn(Cc3cc(F)cc(C(F)(F)F)c3F)c2n1 ; CC1(C)OB(c2cc(C#N)cc(C#N)c2)OC1(C)C ; N#Cc1cc(C#N)cc(-c2nc(N)nc3c2ncn3Cc2cc(F)cc(C(F)(F)F)c2F)c1]

The compound of Example 65 (300 mg, 12.89% yield) was obtained as a yellow solid in the same manner as in Example 63 using (2,5-difluoro-3-(trifluoromethyl)phenyl)methanol (3.00 g, 14.07 mmol) as a starting material. ¹H NMR (400 MHz, DMSO-d6) δ: 9.31 (s, 2H), 8.65 (s, 1H), 8.38 (s, 1H), 7.78-7.74 (m, 1H), 7.49-7.45 (m, 1H), 6.84 (s, 2H), 5.50 (s, 2H); MS: m/z=456.0 (M+1, ESI+).

### Example 66: 3-(2-amino-9-(2,5-difluoro-3-(trifluoromethyl)benzyl)-9H-purin-6-yl)-5-cyanobenzamide

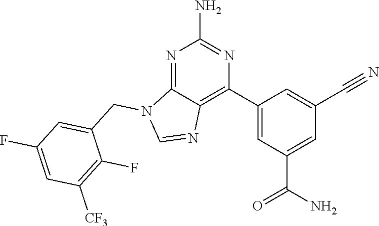 [SMILES: N#Cc1cc(C(N)=O)cc(-c2nc(N)nc3c2ncn3Cc2cc(F)cc(C(F)(F)F)c2F)c1]

The compound of Example 65 (80 mg, 0.18 mmol) was dissolved in toluene (1 mL), and then acetaldoxime (52 mg, 0.88 mmol) and InCl₃ (4 mg, 0.02 mmol) were added. Thereafter, the mixture was stirred at 90° C. for 50 minutes. The mixture was concentrated to afford a residue, which was purified by pre-HPLC (ACN/H₂O=40/60) to afford the compound of Example 66 (20 mg, 23.47%) as a white solid. ¹H NMR (400 MHz, DMSO-d6) δ: 9.43-9.42 (m, 1H), 9.26-9.25 (m, 1H), 8.44-8.43 (m, 1H), 8.35 (s, 1H), 8.25 (s, 1H), 7.78-7.74 (m, 2H), 7.48-7.44 (m, 1H), 6.79 (s, 2H), 5.50 (s, 2H); MS: m/z=474.0 (M+1, ESI+).

### Example 67: 5-(2-amino-9-(5-chloro-2-fluoro-3-(trifluoromethyl)benzyl)-9H-purin-6-yl)isophthalonitrile

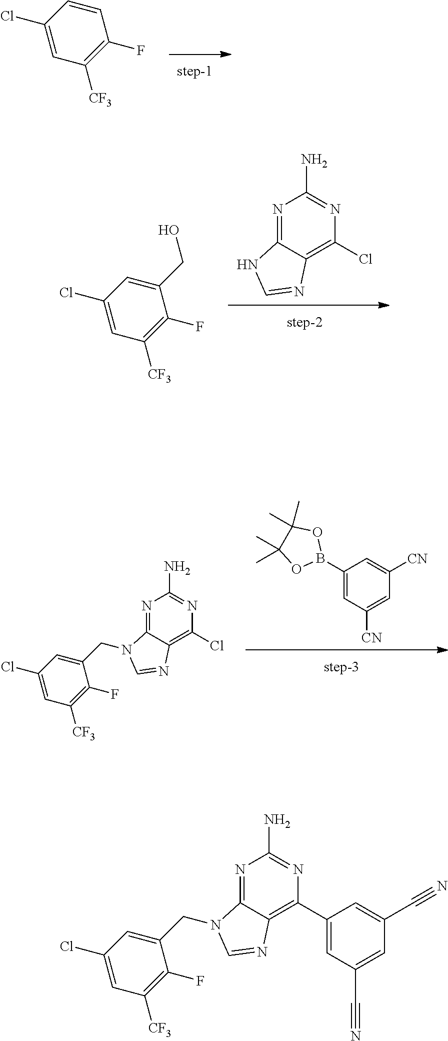 [SMILES: Fc1ccc(Cl)cc1C(F)(F)F ; OCc1cc(Cl)cc(C(F)(F)F)c1F ; Nc1nc(Cl)c2nc[nH]c2n1 ; Nc1nc(Cl)c2ncn(Cc3cc(Cl)cc(C(F)(F)F)c3F)c2n1 ; CC1(C)OB(c2cc(C#N)cc(C#N)c2)OC1(C)C ; N#Cc1cc(C#N)cc(-c2nc(N)nc3c2ncn3Cc2cc(Cl)cc(C(F)(F)F)c2F)c1]

### Step 1: (5-chloro-2-fluoro-3-(trifluoromethyl)phenyl)methanol

4-chloro-1-fluoro-2-(trifluoromethyl)benzene (2100 mg, 10.57 mmol) was dissolved in THF (15 mL) under nitrogen atmosphere. TMEDA (1843 mg, 15.86 mmol) and n-BuLi (5.5 mL, 13.75 mmol) were slowly added at −78° C., and the mixture was stirred for 1 hour. Thereafter, DMF (1082 mg, 14.80 mmol) was added, and the mixture was stirred at 25° C. for 0.5 hours. NaBH₄ (799 mg, 21.15 mmol) was then added, and the mixture was stirred at 25° C. for an additional 0.5 hours. Water (200 mL) was poured onto the resulting mixture, which was then extracted three times with EtOAc (400 mL). The combined organic layer was dried over anhydrous Na₂SO₄ and concentrated to afford (5-chloro-2-fluoro-3-(trifluoromethyl)phenyl)methanol (2400 mg, 99.27% yield) as a yellow oil. ¹H NMR (300 MHz, DMSO-d6) δ: 7.84-7.79 (m, 2H), 5.67-5.63 (m, 1H), 4.64 (d, 2H).

### Step 2: 6-chloro-9-(5-chloro-2-fluoro-3-(trifluoromethyl)benzyl)-9H-purin-2-amine

(5-chloro-2-fluoro-3-(trifluoromethyl)phenyl)methanol (2.4 g, 10.50 mmol) was dissolved in DCM (30 mL) at 0° C. under nitrogen atmosphere, and TsCl (2.16 g, 11.34 mmol) and TEA (3.28 g, 32.4 mmol) were added. The reaction mixture was stirred at 0° C. for 1 hour, and then 6-chloro-9H-purin-2-amine (3.63 g, 21.4 mmol) and K₂CO₃ (4.44 g, 32.1 mmol) were added. The mixture was stirred for an additional hour at 40° C., and then the solvent was removed, and the residue was purified by silica gel column chromatography (DCM:MeOH=25:1 to 10:1) to afford 6-chloro-9-(5-chloro-2-fluoro-3-(trifluoromethyl)benzyl)-9H-purin-2-amine (1.6 g, 39.25% yield) as a white solid. MS: m/z=379.80 (M+1, ESI+).

### Step 3: 5-(2-amino-9-(5-chloro-2-fluoro-3-(trifluoromethyl)benzyl)-9H-purin-6-yl)isophthalonitrile

6-chloro-9-(5-chloro-2-fluoro-3-(trifluoromethyl)benzyl)-9H-purin-2-amine (300 mg, 0.78 mmol), 5-(4,4,5,5-tetramethyl-1,3,2-dioxaborolan-2-yl)isophthalonitrile (970 mg, 1.18 mmol), K₂CO₃ (327 mg, 2.36 mmol), and Pd(PPh₃)₄ (136 mg, 0.11 mmol) were dissolved in 1,4-dioxane/H₂O (4/1) (5 mL), and the mixture was stirred at 100° C. for 2 hours under nitrogen atmosphere. The mixture was filtered, concentrated in vacuo, washed with DCM (20 mL×2), and concentrated in vacuo to afford the compound of Example 67 (60 mg, 1⁶0.11% yield) as a white solid. ¹H NMR (400 MHz, DMSO-d6) δ: 9.31 (d, 2H), 8.64 (s, 1H), 8.39 (s, 1H), 7.92-7.90 (m, 1H), 7.66-7.64 (m, 1H), 6.85 (s, 2H), 5.50 (s, 2H). MS: m/z=471.9 (M+1, ESI+).

### Example 68: 3-(2-amino-9-(5-chloro-2-fluoro-3-(trifluoromethyl)benzyl)-9H-purin-6-yl)-5-cyanobenzamide

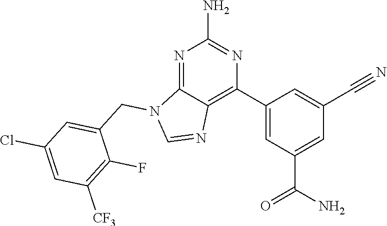 [SMILES: N#Cc1cc(C(N)=O)cc(-c2nc(N)nc3c2ncn3Cc2cc(Cl)cc(C(F)(F)F)c2F)c1]

The compound of Example 67 (300 mg, 0.63 mmol), acetaldoxime (247 mg, 4.19 mmol), and InCl₃ (18 mg, 0.08 mmol) were dissolved in 1,4-dioxane/toluene (1/1) (5 mL), and then the mixture was stirred at 90° C. for 1 hour under nitrogen (argon or air). Thereafter, the mixture was filtered, concentrated in vacuo, washed with DCM (20 mL×2), and concentrated to afford the compound of Example 68 (80 mg, 25.72% yield) as a white solid. ¹H NMR (400 MHz, DMSO-d6) δ: 9.43 (s, 1H), 9.25 (s, 1H), 8.44 (s, 1H), 8.36 (s, 1H), 8.26 (s, 1H), 7.91 (d, 1H), 7.75 (s, 1H), 7.65 (d, 1H), 6.80 (s, 2H), 5.50 (s, 2H). MS: m/z=490.0 (M+1, ESI+).

### Experimental Example 1: In Vitro Testing for Human Chemokine GPCR Cell-Based Agonist Arrestin Assay

The agonist activity of the compounds of the examples of the present disclosure against the human ACKR3 (CXCR7) receptor was measured in a GPCR cell-based assay in stably transfected CHO-K1 cells (PathHunter® CHO-K1 CXCR7 (CMKOR1) β-Arrestin Cell Line, Cat no. 93-0248C₂). The cells were seeded in a 384-well microplate in a total volume of 20 μL and cultured overnight at 37° C. prior to testing. For agonist measurement, the cells were cultured with the sample to induce a response. An intermediate dilution of the sample stock was performed to generate a 5× sample in assay buffer. 5 μL of the 5× sample was added to the cells and cultured at 37° C. for 120 minutes. The final assay vehicle concentration was 1%.

The results are presented in Table 2 below as efficacy percentage ratings for the maximum response to the control ligand (CXCL12). The efficacy percentage ratings shown in Table 2 are as follows:
A: Efficacy percentage≥50%, B: 20≤Efficacy percentage<50%, C: Efficacy percentage<20%

TABLE 2

| | Activation % | | Activation % |
|---|---|---|---|
| Example | @ 0.1 μM | Example | @ 0.1 μM |
| 1 | A | 2 | A |
| 3 | B | 4 | A |
| 5 | A | 6 | A |
| 7 | B | 8 | C |
| 9 | C | 10 | A |
| 11 | C | 13 | C |
| 14 | C | 15 | C |
| 16 | C | 17 | A |
| 18 | A | 19 | B |
| 20 | A | 21 | A |
| 22 | A | 23 | A |
| 25 | C | 26 | C |
| 29 | C | 30 | C |
| 32 | C | 33 | A |
| 34 | B | 35 | C |
| 36 | C | 37 | B |
| 38 | B | 39 | C |
| 40 | C | 41 | C |
| 42 | C | 43 | C |
| 44 | C | 45 | C |
| 46 | C | 47 | C |
| 48 | C | 49 | C |
| 50 | C | 51 | C |
| 52 | C | 53 | A |
| 54 | A | 55 | A |
| 56 | B | 57 | C |
| 58 | A | 59 | A |
| 60 | A | 61 | A |
| 62 | A | 63 | A |
| 64 | A | 65 | A |
| 66 | A | 67 | A |
| 68 | A | | |

The experimental results in Table 2 confirm that the compounds of the present disclosure act as CXCR7 agonists, and in particular, Examples 1, 2, 4, 5, 6, 10, 17, 18, 20, 21, 22, 23, 33, 53, 54, 55, 58, 59, 60, 61, 62, 63, 64, 65, 66, 67, and 68 exhibited excellent efficacy.

### Experimental Example 2: Bleomycin-Induced Idiopathic Pulmonary Fibrosis Test

Male C57BL/6N Tac mice were administered with the compound of Example 1 and pirfenidone starting on day 0 and the experiment was conducted for 21 days. One hour after drug administration, the animals were anesthetized with ketamine+xylazine (70+5 mg/kg, intravenous injection). An endotracheal catheter was inserted through the mouse's mouth, and a solution of bleomycin (1 mg, 1.5 to 2 U/kg based on a 50 μL mouse dose) was added using a micropipette. The solution was allowed to be absorbed into the lungs. An additional 300 μL of air was then injected to ensure even distribution of the BLM solution.

The compound of Example 1 (10, 30, and 60 mg/kg, oral administration, once daily) and the drug Pirfenidone (100 mg/kg, oral administration, twice daily) were administered for 21 days. The mice were euthanized, and then bronchoalveolar lavage fluid (BALF) was collected. The slides were prepared, and the number of each cell was evaluated. The lung tissues of the euthanized mice were collected, immediately frozen in liquid nitrogen, and stored at −80° C. Thereafter, a portion of the lung lobe was used for hydroxyproline analysis. Hydroxyproline levels were evaluated by hydrolyzing the lung tissues of each mouse with 12 N HCl (1:20) in an oven at 110° C., oxidizing with 0.2 M chloramine-T, reacting with Ehrlich's reagent, and measuring the color change using a spectrophotometer at 550 nm. For histological examination, lung tissue (left lobe) was collected, fixed in 10% NBF for 24 hours, embedded in paraffin wax, and sectioned at 5-micrometer thickness. Lung tissue sections were stained with H&E for light microscopic analysis, and fibrosis grade was assessed using the Ashcroft Fibrosis Score Scale (Hubner et al., 2008).

TABLE 3

| Fibrosis | |
|---|---|
| Grade | Ashcroft scale |
| 0 | Normal Lung |
| 1 | Minimal Fibrous Thickening of Alveolar |
| | or Bronchiolar Walls |
| 2 | — |
| 3 | Moderate Thickening of Walls without Obvious |
| | Damage to Lung Architecture |
| 4 | — |
| 5 | Increased Fibrosis with Definite Damage |
| | to Lung Structure and Formation of |
| | Fibrous Bands or Small Fibrous Masses |
| 6 | — |
| 7 | Severe Distortion of Structure and Large Fibrous |
| | Areas; “Honeycomb Lung” is Placed in this Category |
| 8 | Total Fibrous Obliteration of Lung Fields |

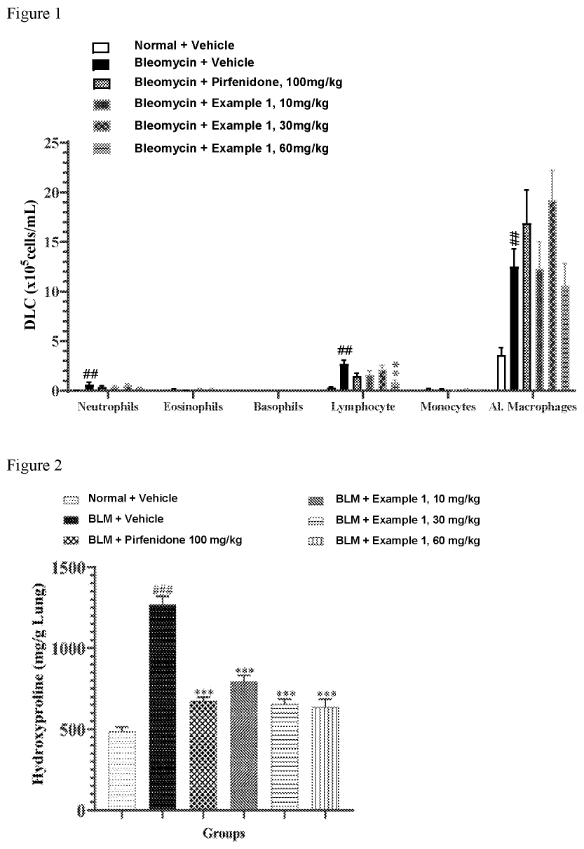

FIG. 1 shows the effect of the compound of the present invention on BALF absolute differential cell count in a bleomycin-induced mouse IPF model. As shown in FIG. 1, macrophages, lymphocytes, and neutrophils were significantly increased in the BALF of the bleomycin-treated disease control group compared to the normal control group. In contrast, the number of lymphocytes was decreased in the groups administered with the compound of Example 1 (10, 30, and 60 mg/kg), and a significant decrease was observed particularly in the group administered at 60 mg/kg.

FIG. 2 is a diagram showing the effect of the compound of Example 1 on lung hydroxyproline in a bleomycin-induced IPF model. The hydroxyproline content is known to be an indicator of collagen deposition in fibrotic tissue. As shown in FIG. 2, the lung hydroxyproline content was significantly increased in the bleomycin-treated disease control group compared to the normal control group. In contrast, the hydroxyproline content was significantly decreased in the groups administered with the compound of Example 1 (10, 30, and 60 mg/kg).

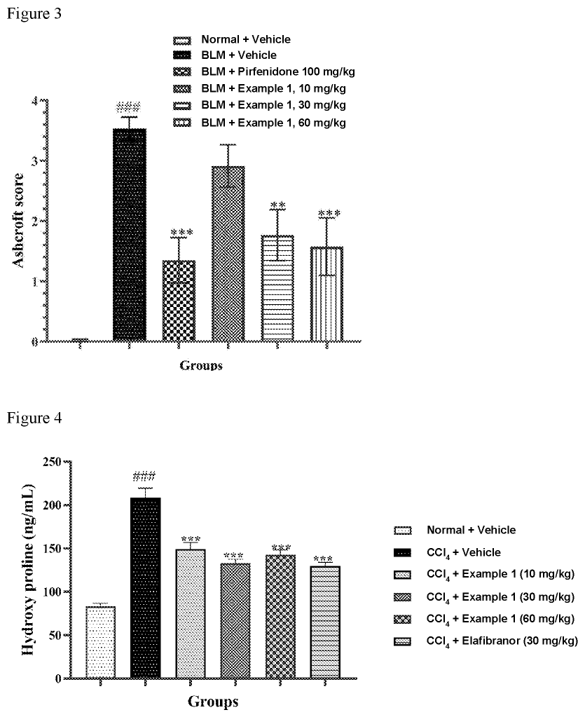

FIG. 3 is a diagram showing the effect of the compound of Example 1 on the Ashcroft fibrosis score in a bleomycin-induced IPF model. FIG. 3 shows that the Ashcroft score in the bleomycin-treated diseased control group was significantly increased compared to the normal control group, indicating significant progression of fibrosis 21 days after bleomycin administration. Meanwhile, the fibrosis score was decreased in the groups administered with the compound of Example 1 (10, 30, and 60 mg/kg) compared to the disease control group, and statistically significant decreases were observed particularly in the groups administered at 30 mg/kg and 60 mg/kg.

### Experimental Example 3: Carbon Tetrachloride (CCl4)-Induced Liver Fibrosis Test

Male C57BL/6N Tac mice were injected intraperitoneally twice weekly for 6 weeks with a mixture of carbon tetrachloride (CCl₄) and olive oil (3:1 ratio, 25% CCl₄ in olive oil). The compound of Example 1 (10, 30, and 60 mg/kg, oral administration, once daily), elafibranor (30 mg/kg, oral administration, once daily) were administered for 6 weeks starting on day 0, prior to CCl₄ administration. After the end of the test, the mice were euthanized, and serum and liver samples were collected for further analysis. The collected serum was separated and stored at the temperature of −80° C., and then liver enzyme analysis, including ALT levels, was performed. For hydroxyproline analysis, liver sample pieces were flash frozen in liquid nitrogen and stored at −80° C. Each liver tissue of about 50 mg was hydrolyzed with 12N HCl (1:20) at 111° C. for 20 hours. The hydrolyzate was centrifuged (14,000 RPM, 15 min), and the supernatant was separated. In a 96-well plate, 15 μL of supernatant and 100 μL of chloramine T were cultured for 20 minutes. Thereafter, 100 μL of Ehrlich's solution was added and cultured at 75° C. for 30 minutes in the dark. The final absorbance of the plate was measured at 550 nm to assess the hydroxyproline concentration.

The liver tissue was collected, preserved in 10% neutral buffered formalin for 24 hours, embedded in paraffin wax, and then sectioned into 5 m-thick sections. After H&E staining, the degree of fibrosis was assessed according to the Ishak fibrosis grade (see Table 4 below).

TABLE 4

| Fibrosis Grade | Ishak grade: Category description |
|---|---|
| 0 | No Fibrosis |
| 1 | Fibrous expansion of some portal |
| | areas ± short fibrous septa |
| 2 | Fibrous expansion of most of the portal |
| | areas ± short fibrous septa |
| 3 | Fibrous expansion of most of the portal areas |
| | with occasional portal to portal (P-P) bridging |
| 4 | Fibrous expansion of most of the portal areas |
| | with marked bridging portal to portal(P-P) |
| | as well as portal to central(P-C) |
| 5 | Marked bridging (P-P and/or P-C), with |
| | occasional nodules (incomplete nodules) |
| 6 | Cirrhosis, probable or indefinite |

The results of measuring hepatic hydroxyproline in a carbon tetrachloride-induced liver fibrosis model are shown in FIG. 4. The hydroxyproline content is known to be an indicator of collagen deposition in fibrotic tissue. As shown in FIG. 4, the hepatic hydroxyproline content was significantly increased in the disease control group treated with CCl₄+vehicle compared to the normal control group. In contrast, the hepatic hydroxyproline content was significantly decreased in the groups administered with the compound of Example 1 (10, 30, and 60 mg/kg) compared to the disease control group, and an effect equivalent to elafibranor was shown.

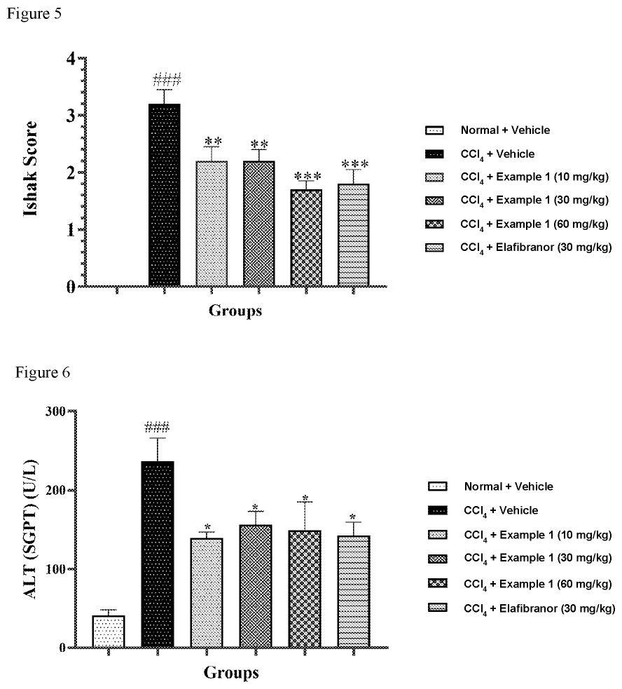

In addition, FIG. 5 shows the results of measuring Ishak fibrosis score in a carbon tetrachloride-induced liver fibrosis model. As shown in FIG. 5, the Ishak fibrosis score in H&E-stained liver sections from the disease control group was increased significantly after 6 weeks of CCl₄ administration compared to the normal control group, indicating significant progression of fibrosis. However, the fibrosis score was significantly decreased in the groups administered with the compound of Example 1 (10, 30, and 60 mg/kg) compared to the disease control group.

Furthermore, the results of measuring ALT in a carbon tetrachloride-induced liver fibrosis model (FIG. 6) confirmed that the ALT level, which was sharply increased in the disease control group compared to the normal control group, was significantly reduced by administration of the compound of Example 1 (10, 30, and 60 mg/kg).

### Experimental Example 4: MOG35-55-Induced EAE Model Test

Myelin oligodendrocyte glycoprotein (MOG35-55) was dissolved in saline at a concentration of 2 mg/mL, and heat-killed Mycobacterium tuberculosis was added to prepare a complete Freund's adjuvant at a final concentration of 4 mg/mL. A2 mg/mL MOG 35-55 solution was emulsified with 4 mg/mL CFA of the same volume using a high-speed homogenizer at 30,000 rpm on ice for 1.5 hours to form an emulsion. Male C57BL/6N Tac mice were anesthetized with 14% isoflurane. Thereafter, 100 μL of the emulsion was injected subcutaneously at three sites: one site along the midline of the back between the shoulders and two sites on each side of the midline of the waist. 48 hours later, disease was induced in all mice by intraperitoneal injection of pertussis toxin (200 ng in 200 μL of PBS). The compounds of Examples 1, 2, and 4 at a dose of 30 mg/kg were administered orally for 21 days.

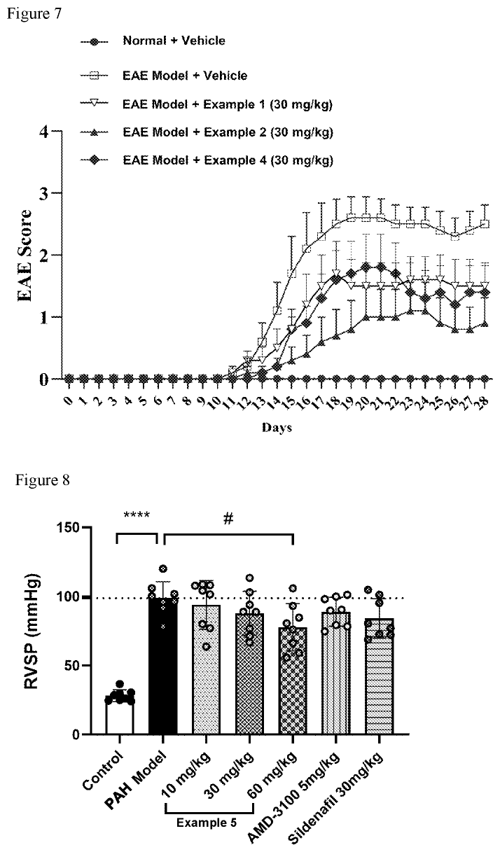

The EAE clinical signs were scored according to the EAE clinical scoring system described in Table 5 below. The results are shown in FIG. 7.

TABLE 5

| Score | Clinical sign |
|---|---|
| 0 | Normal |
| 1 | Dropping tail |
| 2 | Dropping tail, hind limb weakness |
| 3 | Dropping tail, partial hind limb paralysis, gait disturbance |
| 4 | Complete paralysis of both hind limbs |
| 5 | Moribunda or death |

The EAE (Experimental autoimmune encephalomyelitis) mouse model is widely known as a model for autoimmune diseases, particularly multiple sclerosis. The therapeutic effects of the compound of the present invention on autoimmune disease were confirmed in the EAE model. As shown in FIG. 7, the groups administered with the compounds of Examples 1, 2, and 4 showed a significant reduction in clinical signs of neuroinflammation compared to the disease control group (Model-Vehicle).

### Experimental Example 5: MCT (Monocrotaline)-Induced Pulmonary Arterial Hypertension Model Test in Male SD Rats

Male SD rats were administered 60 mg/kg of MCT (monocrotaline) via intraperitoneal injection on Day 1, and the test substance was administered orally once daily from Day 1 to Day 28. The test substance was the compound of Example 5 (10, 30, and 60 mg/kg, po), and Sildenafil (30 mg/kg, po) was used as a positive control substance, and AMD3100 octahydrochloride (CAS155148-31-5) (5 mg/kg, po) was used as a comparison substance. On Day 29, the rats were anesthetized. Thereafter, a PE-50 catheter was inserted into the right jugular vein and slowly advanced into the subclavian vein, right atrium, and right ventricle. Once the right ventricular waveform had been stabilized, right ventricular systolic pressure (RVSP) was measured.

For the right ventricular hypertrophy index (RVH Index), the rats were perfused with PBS through the abdominal aorta, the hearts were isolated, the right ventricle was separated from the interventricular septum along the pulmonary artery orifice, the left ventricle and interventricular septum were excised and washed, and the weight of the right ventricle (RV) and the combined weight of the left ventricle and interventricular septum (LV+S) were measured. The ratio of right ventricular weight to left ventricular plus septal weight (RV/LV+S) and the ratio of right ventricle weight to body weight (RV/BW) were calculated.

For histopathological analysis, the left lung and right ventricle of rats were fixed by perfusion with 10% neutral buffered formalin fixative, sectioned, embedded in paraffin, and stained with H&E. The right ventricular wall thickness was measured at three locations using an optical microscope, and the average of the measurements was used as the right ventricular wall thickness.

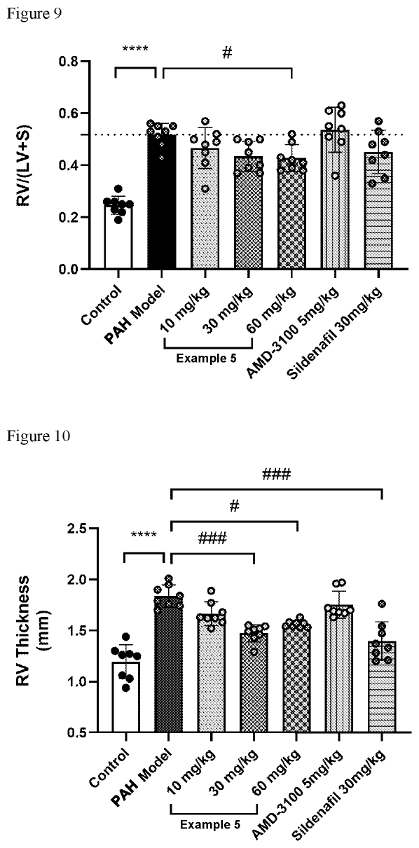

As can be seen in FIGS. 8 to 10, pulmonary artery pressure, i.e. right ventricular systolic pressure (RVSP), was increased in the MCT administration model. However, in the group administered with the compound of Example 5, RVSP was decreased in a dose-dependent manner, and a significant decrease was confirmed in the group administered with the compound of Example 5 (60 mg/kg). No significant changes were observed in the groups administered with AMD3100 and the positive control substance, sildenafil. When pulmonary artery pressure increases, right ventricular load increases, resulting in right ventricular hypertrophy. As a result, in the MCT administration model, the RV/LV+S ratio was significantly increased compared to the control group, and when the compound of Example 5 was administered at doses of 10, 30, and 60 mg/kg, the RV/LV+S ratio was decreased in a dose-dependent manner. In particular, the RV/LV+S ratio was significantly lower in the group administered with the compound of Example 5 at a dose of 60 mg/kg than in the control group. No significant changes were observed in the groups administered with AMD3100 and the positive control, sildenafil. The right ventricular wall thickness was significantly thickened in the MCT model, and the groups administered with the compound of Example 5 at doses of 30 mg/kg and 60 mg/kg showed a significant decrease in right ventricular wall thickness. Meanwhile, AMD3100 showed no significant decrease, while the positive control, sildenafil, significantly reduced the right ventricular wall thickness.

### Experimental Example 6: CXCL12-Induced Breast Cancer (MDA-MB-231, TNBC) Metastasis Model Test

Twenty-four Oris™ cell seeding stoppers were placed in a 96-well plate. MDA-MB-231 cells were suspended in DMEM to adjust the cell count to 5×10⁵ cells/mL. Thereafter, 100 μL of cells were pipetted into each test well through one of the side ports of the Oris™ cell seeding stoppers. The seeding plate containing the Oris™ cell seeding stoppers was cultured in a humidified chamber (37° C., 5% CO₂) for 24 hours. After 24 hours, the stoppers were removed, and 100 μL of fresh medium with or without CXCL12 (100 ng/mL) was added. The compounds of Examples 1, 2, and 4 were each added at a concentration of 1 M and cultured in a humidified chamber (37° C., 5% CO₂) for 28 hours to determine cell migration. After incubation, the migrated cells were stained with Calcein-AM (1 M) for 30 minutes, and the cell migration was photographed using a Leica DMI3000 B. The images were calculated using ImageJ, and data were analyzed using GraphPad Prism.

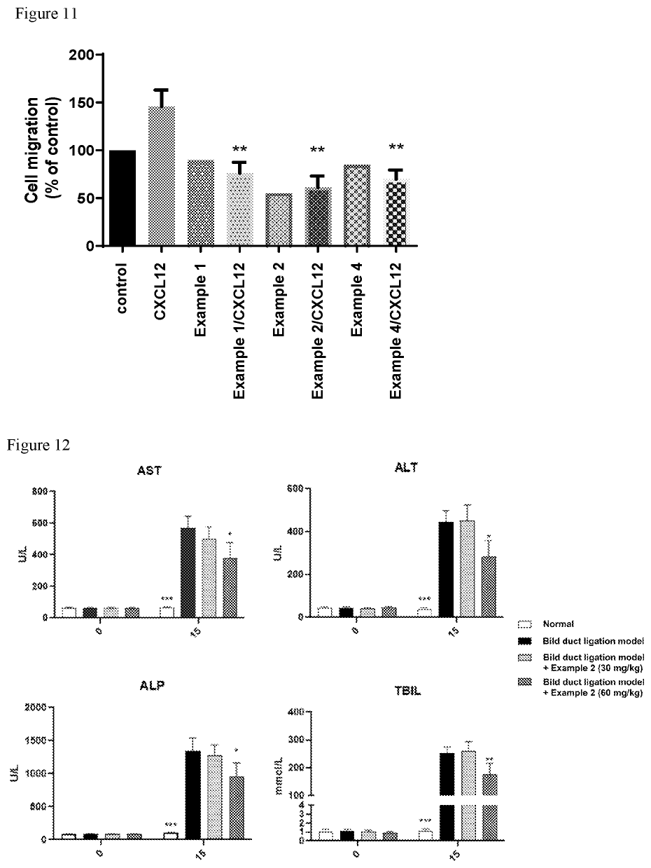

As shown in FIG. 11, cell migration was increased in breast cancer cells treated with CXCL12. However, it was confirmed that cell migration was inhibited in the group administered with the compounds of Examples 1, 2, and 4 at a concentration of 1 M. These results demonstrate that the compounds of the present invention can inhibit cancer metastasis by inhibiting cell migration.

### Experimental Example 7: Bile Duct Ligation-Induced Cholestatic Liver Disease Model Test in Male C57BL/6J Mice

On Day 0, serum was collected from male C57BL/6J mice fasted for one day for evaluation of AST, ALT, ALP, and total bilirubin. On Day 1, male C57BL/6J mice were anesthetized with isoflurane, and the common bile duct was exposed 1 cm through a midline upper abdominal incision, ligated twice with 5/0 silk sutures, and an incision was made between them. After surgery, the muscles and skin were sutured separately with 4/0 silk. In the normal group, only exploratory laparotomy was performed after the midline incision and suturing was performed. From Day 2 to Day 15, the compound of Example 2 (30 mg/kg, 60 mg/kg) was administered orally once daily for 2 weeks. On Day 15, the mice were euthanized with CO₂, blood was collected from the heart, and the serum was isolated. AST, ALT, ALP, and total bilirubin in the serum were evaluated. The liver tissues were isolated and weighed, and the mesenchymal portion was fixed in 1000 NBF and evaluated by hematoxylin and eosin (H&E) staining and aL-SMA immunohistochemistry (tIC). For H&E staining, the liver sections were cut into 4 m thick, dried in an oven for 1 hour, and then stained with an integrated workstation Leica SPECTRA ST (HistoCore SPECTRA H&E Staining System, S1 #3801654, Lot #050223). For histological evaluation, H&E-stained images were scanned using LEICA Aperio GT450, and the scores of ductal proliferation, portal inflammation, and confluent necrosis were determined using the criteria listed in Table 6 below.

TABLE 6

| Score | Ductal proliferation | Portal inflammation | Confluent necrosis |
|---|---|---|---|
| 0 | No ductular | None | Absent |
| | proliferation | | |
| 1 | Ductular proliferation | Mild, some or all | Focal confluent necrosis |
| | limited in the portal | portal areas | |
| | areas | | |
| 2 | Septal ductular | Moderate, some or all | Zone 3 necrosis in some areas |
| | proliferation | portal areas | |
| 3 | Incomplete nodule | Moderate/marked, all | Zone 3 necrosis in most areas |
| | formation | portal areas | |
| 4 | Complete nodule | Marked, all portal | Zone 3 necrosis + occasional |
| | formation | areas | portal-central (P-C) bridging |
| 5 | \ | \ | Zone 3 necrosis + multiple P-C |
| | | | bridging |
| 6 | \ | \ | Panacinar or multiacinar necrosis |

Immunohistochemical staining was performed by incubating primary rabbit polyclonal anti-α-SMA antibody (Abcam #ab5694) at a 1:400 dilution for 1 hour, then adding anti-rabbit IgG (H+L) (Jackson immuno #111-035-045) at a 1:500 dilution, and scanning the α-SMA-stained sections using a Leica Aperio GT450 scanner for analysis.

As shown in FIG. 12, the blood analysis results on Day 15 showed that the levels of ALT, AST, ALP, and total bilirubin were significantly higher in the bile duct ligation model compared to normal animals. However, in the group administered with the compound of Example 2, the levels of ALT, AST, ALP, and total bilirubin were decreased in a dose-dependent manner. In particular, a significant decrease in these levels was observed in the group administered with the compound of Example 2 at a dose of 60 mg/kg.

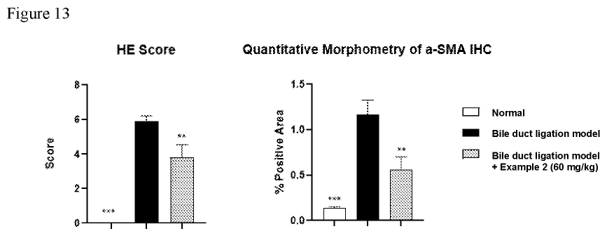

As shown in FIG. 13, liver tissue fibrosis scores were increased in the bile duct ligation model compared to the normal animals in liver tissue stained with H&E solution. However, liver tissue fibrosis was significantly inhibited in the group administered with the compound of Example 2 (60 mg/kg). In addition, the expression of α-SMA, a fibrogenic protein, was significantly increased in the bile duct ligation model. However, the expression of α-SMA was significantly reduced in the group administered with the compound of Example 2 (60 mg/kg).

1. A compound of Formula I below, a stereoisomer, isotopically labeled compound, hydrate, solvate, or pharmaceutically acceptable salt thereof: 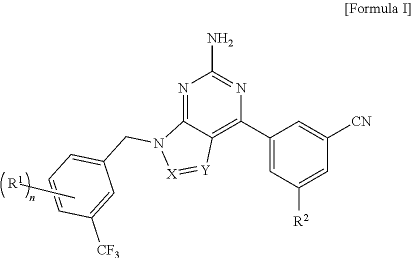
in the above formula,
X and Y are each independently CR^{a} or N;
R¹ is selected from the group consisting of hydrogen, halogen, C₁-C₆ haloalkyl, C₁-C₆ alkyl, C₁-C₆ alkoxy, C₂-C₆ alkenyl, and C₂-C₆ alkynyl;
R² is —CN or —CONR^{b}R^{c};
R^{a}, R^{b}, and R^{c} are each independently H or C₁-C₆ alkyl; and
n is an integer from 0 to 2.

2. The compound, stereoisomer, isotopically labeled compound, hydrate, solvate, or pharmaceutically acceptable salt thereof according to claim 1, wherein R¹ is H, F, Cl, Br, I, CF₃, —CH₃, —OCH₃, or —C≡C.

3. The compound, stereoisomer, isotopically labeled compound, hydrate, solvate, or pharmaceutically acceptable salt thereof according to claim 1, wherein R² is —CN or —CONH₂.

4. The compound, stereoisomer, isotopically labeled compound, hydrate, solvate, or pharmaceutically acceptable salt thereof according to claim 1, wherein the compound is represented by any one of Formulae IA to ID below: 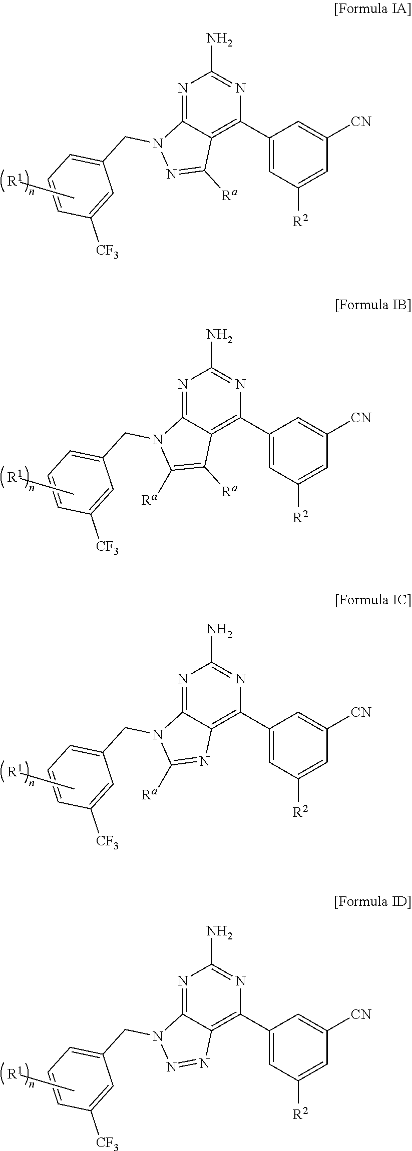
in the above formulae, the definitions of R¹, R², R^{a}, and n are the same as described in claim 1.

5. The compound, stereoisomer, isotopically labeled compound, hydrate, solvate, or pharmaceutically acceptable salt thereof according to claim 1, wherein the compound is represented by Formula I-1 below: 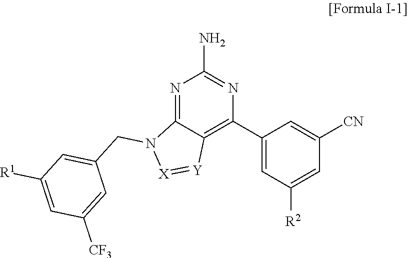
in the above formula, the definitions of X, Y, R¹, and R² are the same as described in claim 1.

6. The compound, stereoisomer, isotopically labeled compound, hydrate, solvate, or pharmaceutically acceptable salt thereof according to claim 1, wherein the compound is selected from the following compounds:

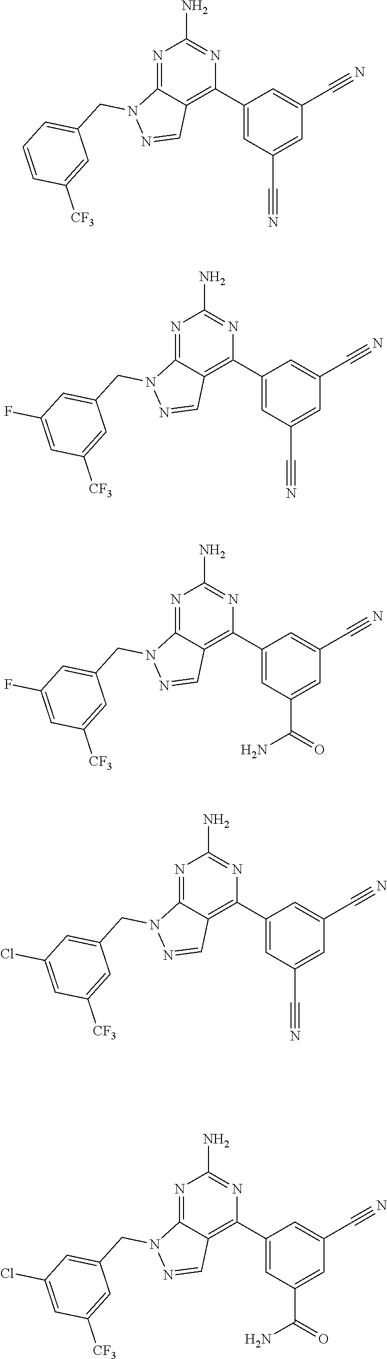 [SMILES: N#Cc1cc(C#N)cc(-c2nc(N)nc3c2cnn3Cc2cccc(C(F)(F)F)c2)c1 ; N#Cc1cc(C#N)cc(-c2nc(N)nc3c2cnn3Cc2cc(F)cc(C(F)(F)F)c2)c1 ; N#Cc1cc(C(N)=O)cc(-c2nc(N)nc3c2cnn3Cc2cc(F)cc(C(F)(F)F)c2)c1 ; N#Cc1cc(C#N)cc(-c2nc(N)nc3c2cnn3Cc2cc(Cl)cc(C(F)(F)F)c2)c1 ; N#Cc1cc(C(N)=O)cc(-c2nc(N)nc3c2cnn3Cc2cc(Cl)cc(C(F)(F)F)c2)c1]

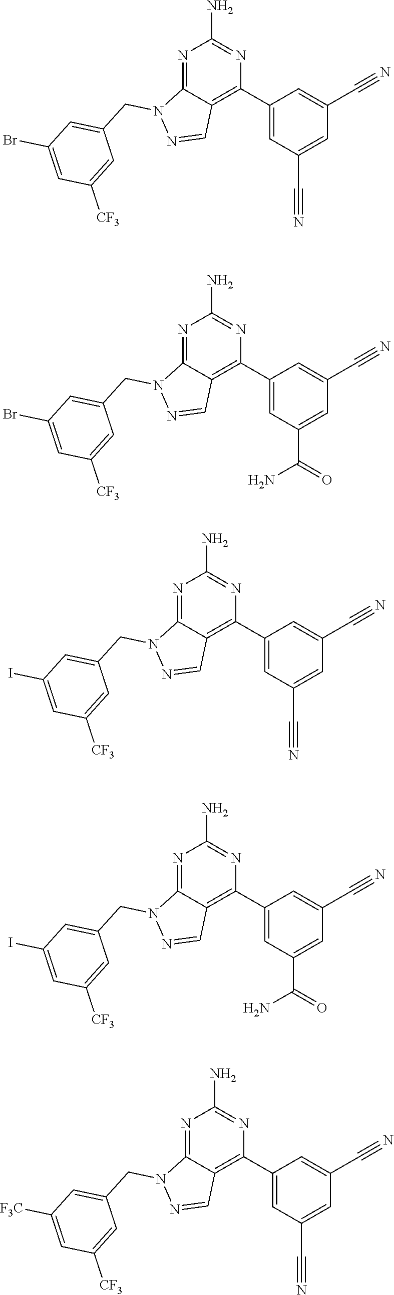 [SMILES: N#Cc1cc(C#N)cc(-c2nc(N)nc3c2cnn3Cc2cc(Br)cc(C(F)(F)F)c2)c1 ; N#Cc1cc(C(N)=O)cc(-c2nc(N)nc3c2cnn3Cc2cc(Br)cc(C(F)(F)F)c2)c1 ; N#Cc1cc(C#N)cc(-c2nc(N)nc3c2cnn3Cc2cc(I)cc(C(F)(F)F)c2)c1 ; N#Cc1cc(C(N)=O)cc(-c2nc(N)nc3c2cnn3Cc2cc(I)cc(C(F)(F)F)c2)c1 ; N#Cc1cc(C#N)cc(-c2nc(N)nc3c2cnn3Cc2cc(C(F)(F)F)cc(C(F)(F)F)c2)c1]

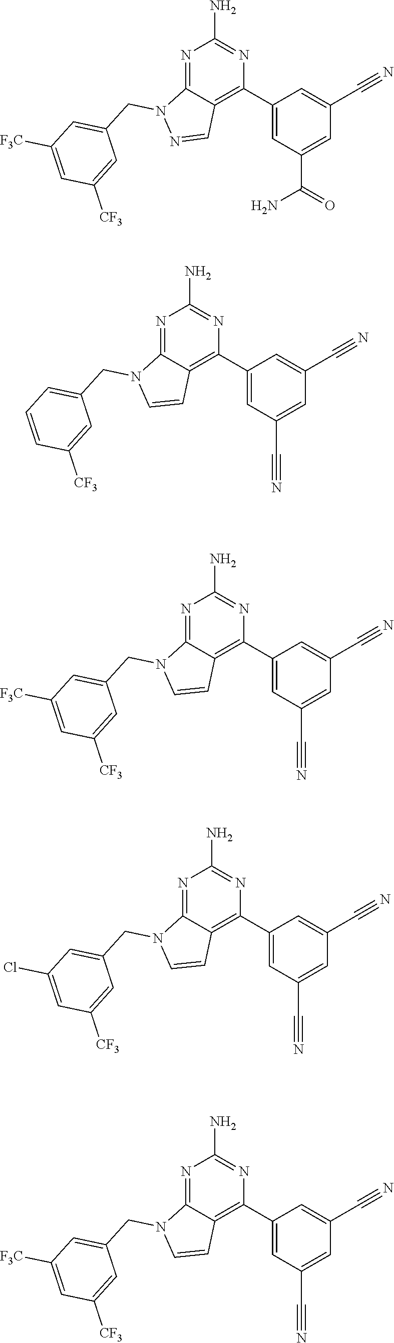 [SMILES: N#Cc1cc(C(N)=O)cc(-c2nc(N)nc3c2cnn3Cc2cc(C(F)(F)F)cc(C(F)(F)F)c2)c1 ; N#Cc1cc(C#N)cc(-c2nc(N)nc3c2ccn3Cc2cccc(C(F)(F)F)c2)c1 ; N#Cc1cc(C#N)cc(-c2nc(N)nc3c2ccn3Cc2cc(C(F)(F)F)cc(C(F)(F)F)c2)c1 ; N#Cc1cc(C#N)cc(-c2nc(N)nc3c2ccn3Cc2cc(Cl)cc(C(F)(F)F)c2)c1 ; N#Cc1cc(C#N)cc(-c2nc(N)nc3c2ccn3Cc2cc(C(F)(F)F)cc(C(F)(F)F)c2)c1]

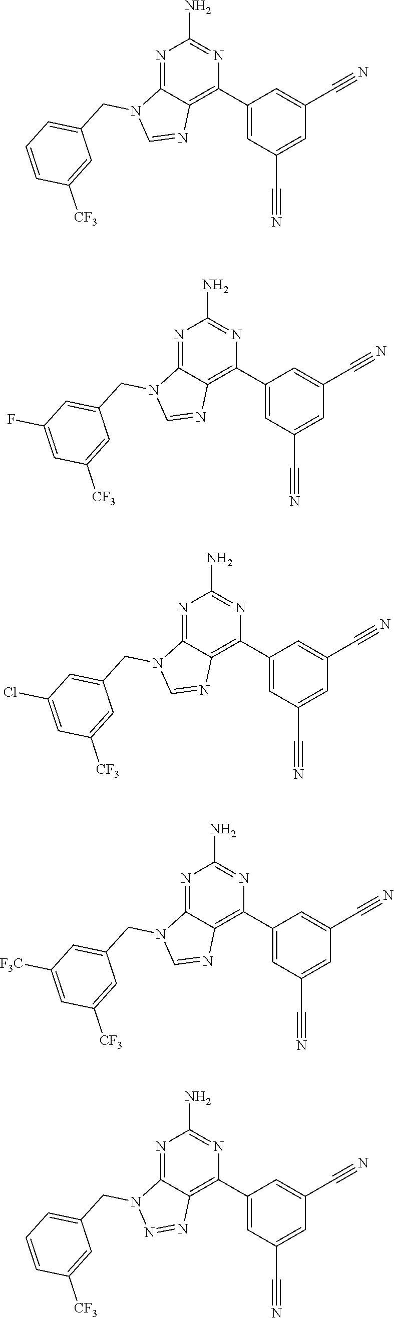 [SMILES: N#Cc1cc(C#N)cc(-c2nc(N)nc3c2ncn3Cc2cccc(C(F)(F)F)c2)c1 ; N#Cc1cc(C#N)cc(-c2nc(N)nc3c2ncn3Cc2cc(F)cc(C(F)(F)F)c2)c1 ; N#Cc1cc(C#N)cc(-c2nc(N)nc3c2ncn3Cc2cc(Cl)cc(C(F)(F)F)c2)c1 ; N#Cc1cc(C#N)cc(-c2nc(N)nc3c2ncn3Cc2cc(C(F)(F)F)cc(C(F)(F)F)c2)c1 ; N#Cc1cc(C#N)cc(-c2nc(N)nc3c2nnn3Cc2cccc(C(F)(F)F)c2)c1]

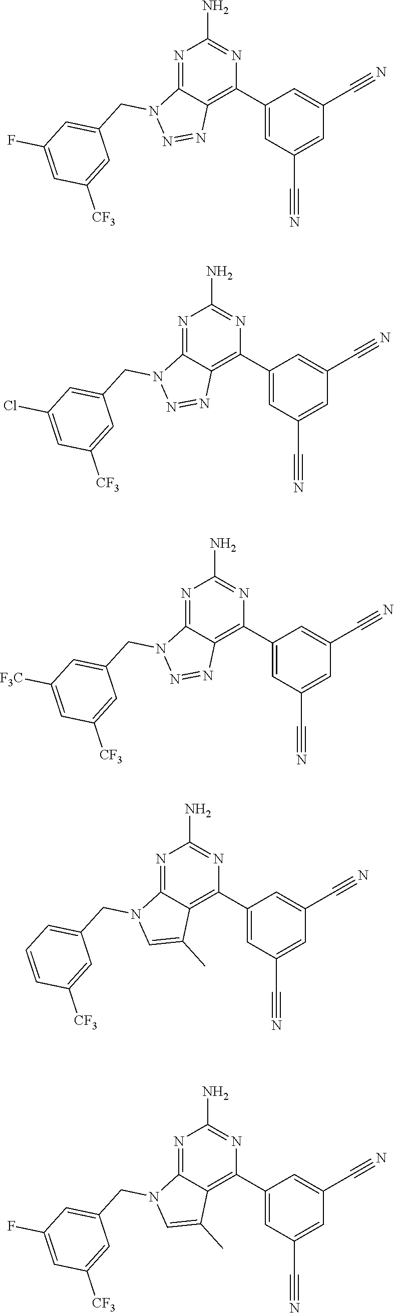 [SMILES: N#Cc1cc(C#N)cc(-c2nc(N)nc3c2nnn3Cc2cc(F)cc(C(F)(F)F)c2)c1 ; N#Cc1cc(C#N)cc(-c2nc(N)nc3c2nnn3Cc2cc(Cl)cc(C(F)(F)F)c2)c1 ; N#Cc1cc(C#N)cc(-c2nc(N)nc3c2nnn3Cc2cc(C(F)(F)F)cc(C(F)(F)F)c2)c1 ; Cc1cn(Cc2cccc(C(F)(F)F)c2)c2nc(N)nc(-c3cc(C#N)cc(C#N)c3)c12 ; Cc1cn(Cc2cc(F)cc(C(F)(F)F)c2)c2nc(N)nc(-c3cc(C#N)cc(C#N)c3)c12]

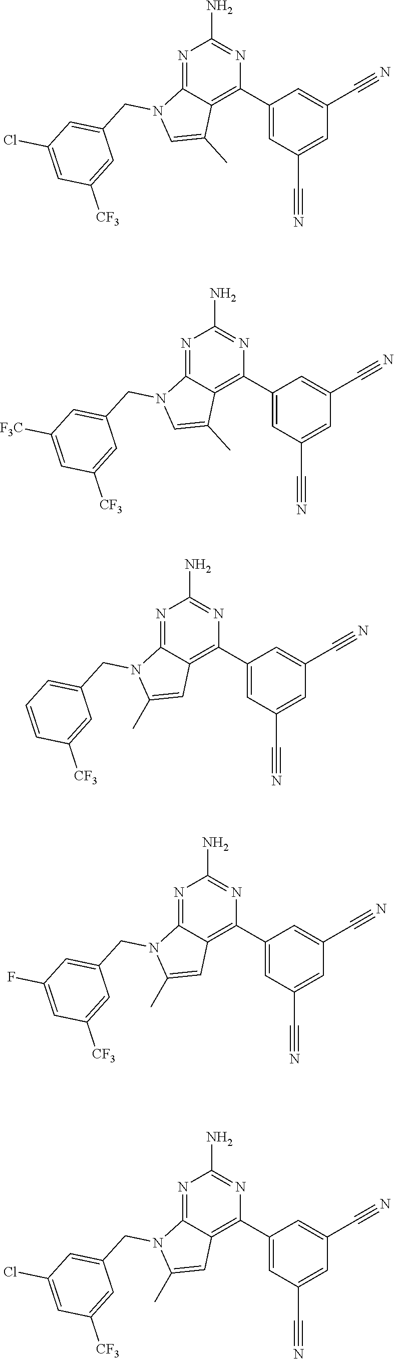 [SMILES: Cc1cn(Cc2cc(Cl)cc(C(F)(F)F)c2)c2nc(N)nc(-c3cc(C#N)cc(C#N)c3)c12 ; Cc1cn(Cc2cc(C(F)(F)F)cc(C(F)(F)F)c2)c2nc(N)nc(-c3cc(C#N)cc(C#N)c3)c12 ; Cc1cc2c(-c3cc(C#N)cc(C#N)c3)nc(N)nc2n1Cc1cccc(C(F)(F)F)c1 ; Cc1cc2c(-c3cc(C#N)cc(C#N)c3)nc(N)nc2n1Cc1cc(F)cc(C(F)(F)F)c1 ; Cc1cc2c(-c3cc(C#N)cc(C#N)c3)nc(N)nc2n1Cc1cc(Cl)cc(C(F)(F)F)c1]

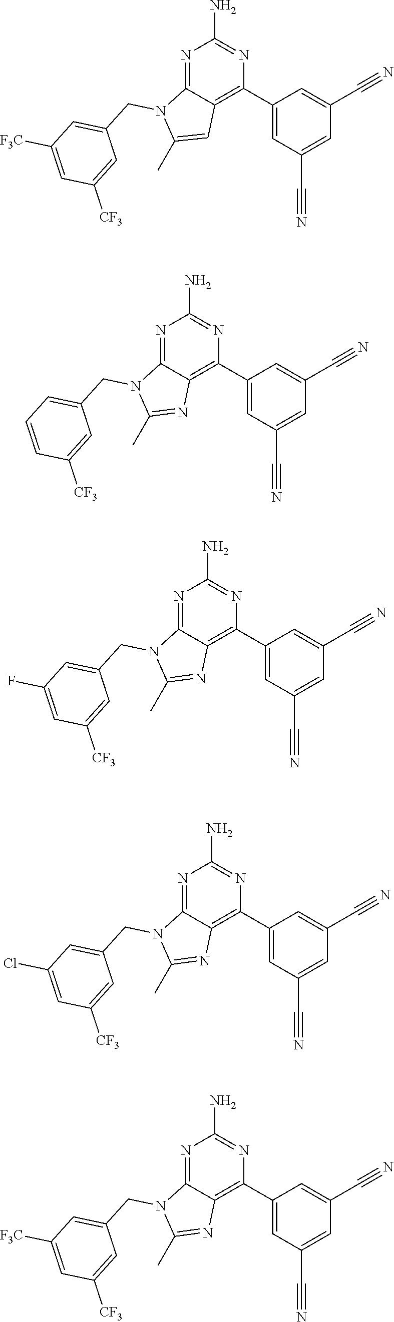 [SMILES: Cc1cc2c(-c3cc(C#N)cc(C#N)c3)nc(N)nc2n1Cc1cc(C(F)(F)F)cc(C(F)(F)F)c1 ; Cc1nc2c(-c3cc(C#N)cc(C#N)c3)nc(N)nc2n1Cc1cccc(C(F)(F)F)c1 ; Cc1nc2c(-c3cc(C#N)cc(C#N)c3)nc(N)nc2n1Cc1cc(F)cc(C(F)(F)F)c1 ; Cc1nc2c(-c3cc(C#N)cc(C#N)c3)nc(N)nc2n1Cc1cc(Cl)cc(C(F)(F)F)c1 ; Cc1nc2c(-c3cc(C#N)cc(C#N)c3)nc(N)nc2n1Cc1cc(C(F)(F)F)cc(C(F)(F)F)c1]

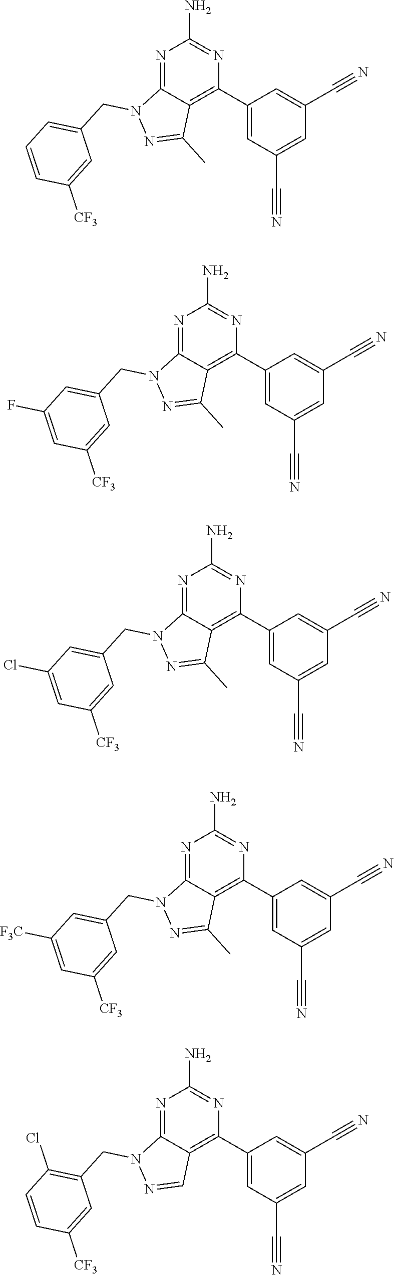 [SMILES: Cc1nn(Cc2cccc(C(F)(F)F)c2)c2nc(N)nc(-c3cc(C#N)cc(C#N)c3)c12 ; Cc1nn(Cc2cc(F)cc(C(F)(F)F)c2)c2nc(N)nc(-c3cc(C#N)cc(C#N)c3)c12 ; Cc1nn(Cc2cc(Cl)cc(C(F)(F)F)c2)c2nc(N)nc(-c3cc(C#N)cc(C#N)c3)c12 ; Cc1nn(Cc2cc(C(F)(F)F)cc(C(F)(F)F)c2)c2nc(N)nc(-c3cc(C#N)cc(C#N)c3)c12 ; N#Cc1cc(C#N)cc(-c2nc(N)nc3c2cnn3Cc2cc(C(F)(F)F)ccc2Cl)c1]

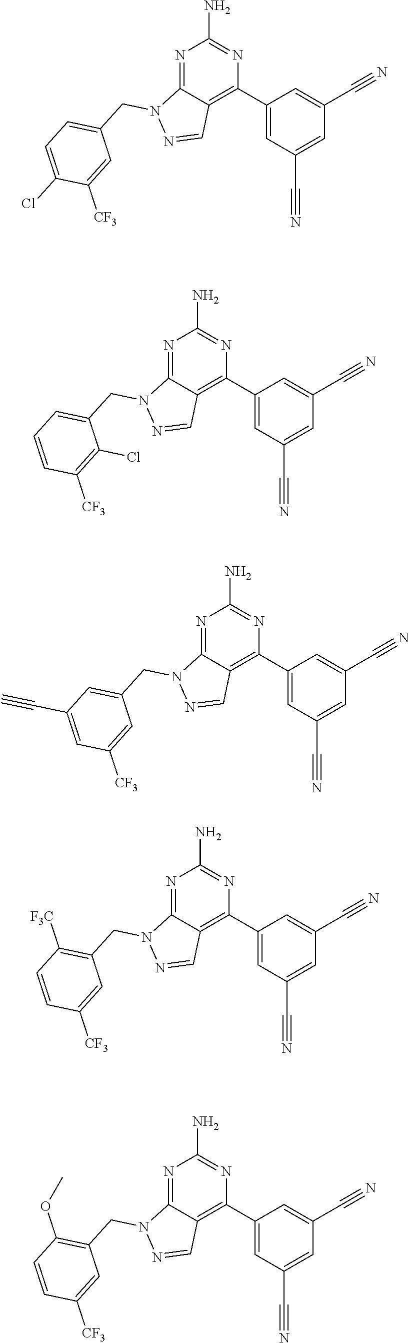 [SMILES: N#Cc1cc(C#N)cc(-c2nc(N)nc3c2cnn3Cc2ccc(Cl)c(C(F)(F)F)c2)c1 ; N#Cc1cc(C#N)cc(-c2nc(N)nc3c2cnn3Cc2cccc(C(F)(F)F)c2Cl)c1 ; C#Cc1cc(Cn2ncc3c(-c4cc(C#N)cc(C#N)c4)nc(N)nc32)cc(C(F)(F)F)c1 ; N#Cc1cc(C#N)cc(-c2nc(N)nc3c2cnn3Cc2cc(C(F)(F)F)ccc2C(F)(F)F)c1 ; COc1ccc(C(F)(F)F)cc1Cn1ncc2c(-c3cc(C#N)cc(C#N)c3)nc(N)nc21]

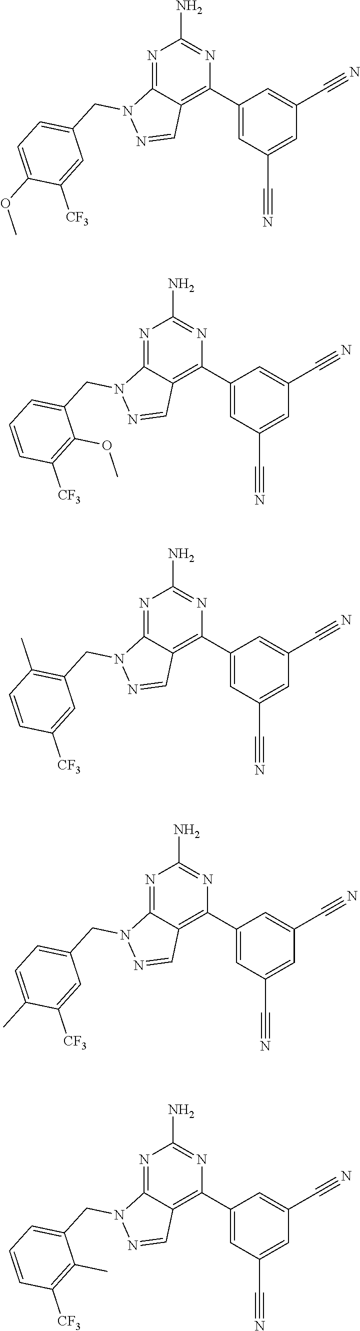 [SMILES: COc1ccc(Cn2ncc3c(-c4cc(C#N)cc(C#N)c4)nc(N)nc32)cc1C(F)(F)F ; COc1c(Cn2ncc3c(-c4cc(C#N)cc(C#N)c4)nc(N)nc32)cccc1C(F)(F)F ; Cc1ccc(C(F)(F)F)cc1Cn1ncc2c(-c3cc(C#N)cc(C#N)c3)nc(N)nc21 ; Cc1ccc(Cn2ncc3c(-c4cc(C#N)cc(C#N)c4)nc(N)nc32)cc1C(F)(F)F ; Cc1c(Cn2ncc3c(-c4cc(C#N)cc(C#N)c4)nc(N)nc32)cccc1C(F)(F)F]

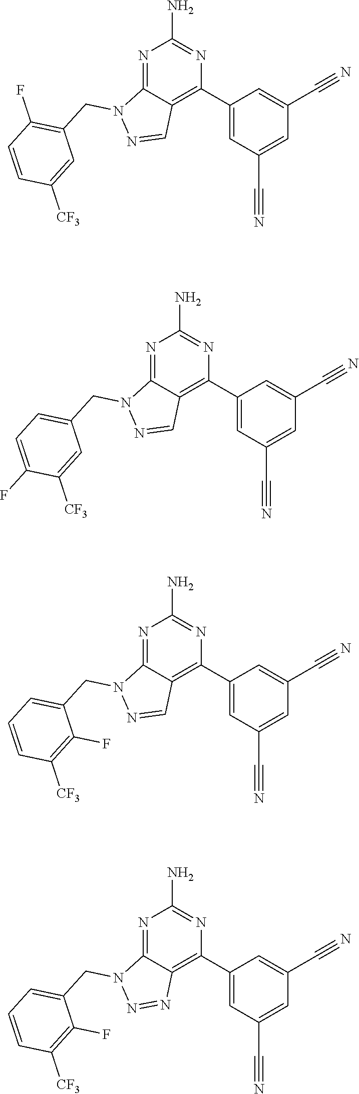 [SMILES: N#Cc1cc(C#N)cc(-c2nc(N)nc3c2cnn3Cc2cc(C(F)(F)F)ccc2F)c1 ; N#Cc1cc(C#N)cc(-c2nc(N)nc3c2cnn3Cc2ccc(F)c(C(F)(F)F)c2)c1 ; N#Cc1cc(C#N)cc(-c2nc(N)nc3c2cnn3Cc2cccc(C(F)(F)F)c2F)c1 ; N#Cc1cc(C#N)cc(-c2nc(N)nc3c2nnn3Cc2cccc(C(F)(F)F)c2F)c1]

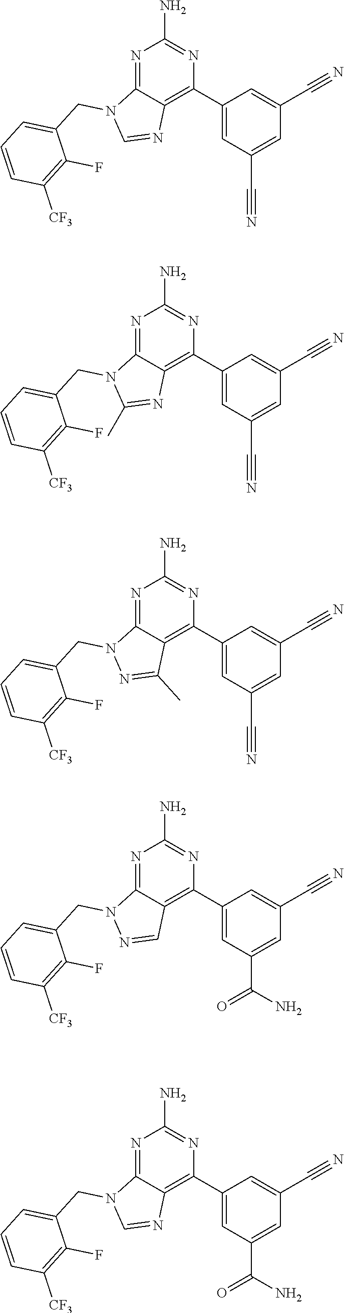 [SMILES: N#Cc1cc(C#N)cc(-c2nc(N)nc3c2ncn3Cc2cccc(C(F)(F)F)c2F)c1 ; Cc1nc2c(-c3cc(C#N)cc(C#N)c3)nc(N)nc2n1Cc1cccc(C(F)(F)F)c1F ; Cc1nn(Cc2cccc(C(F)(F)F)c2F)c2nc(N)nc(-c3cc(C#N)cc(C#N)c3)c12 ; N#Cc1cc(C(N)=O)cc(-c2nc(N)nc3c2cnn3Cc2cccc(C(F)(F)F)c2F)c1 ; N#Cc1cc(C(N)=O)cc(-c2nc(N)nc3c2ncn3Cc2cccc(C(F)(F)F)c2F)c1]

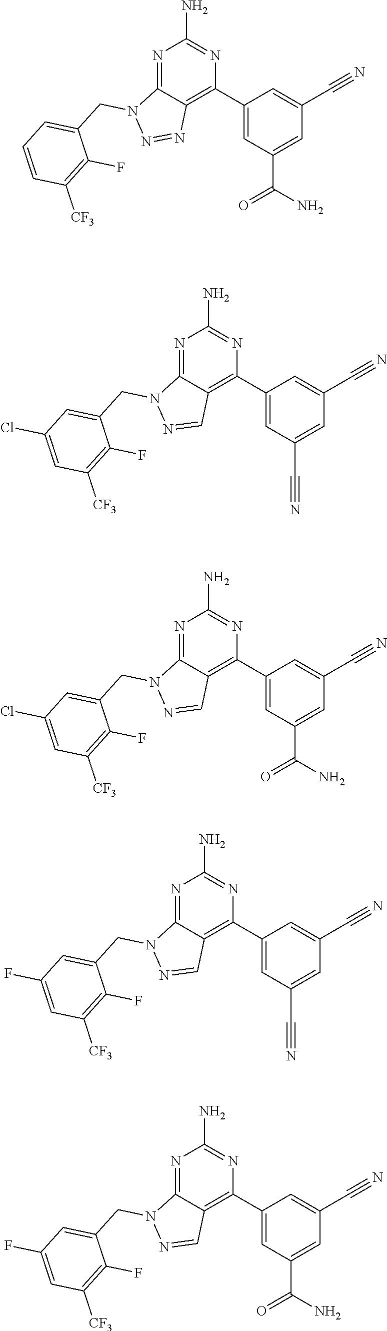 [SMILES: N#Cc1cc(C(N)=O)cc(-c2nc(N)nc3c2nnn3Cc2cccc(C(F)(F)F)c2F)c1 ; N#Cc1cc(C#N)cc(-c2nc(N)nc3c2cnn3Cc2cc(Cl)cc(C(F)(F)F)c2F)c1 ; N#Cc1cc(C(N)=O)cc(-c2nc(N)nc3c2cnn3Cc2cc(Cl)cc(C(F)(F)F)c2F)c1 ; N#Cc1cc(C#N)cc(-c2nc(N)nc3c2cnn3Cc2cc(F)cc(C(F)(F)F)c2F)c1 ; N#Cc1cc(C(N)=O)cc(-c2nc(N)nc3c2cnn3Cc2cc(F)cc(C(F)(F)F)c2F)c1]

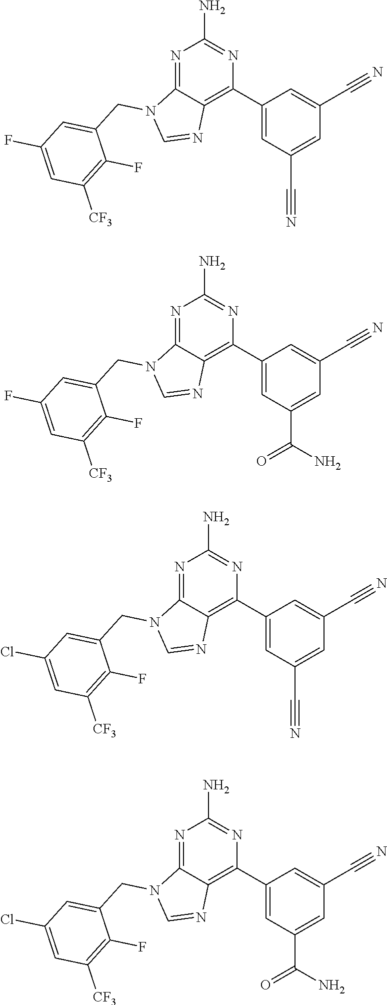 [SMILES: N#Cc1cc(C#N)cc(-c2nc(N)nc3c2ncn3Cc2cc(F)cc(C(F)(F)F)c2F)c1 ; N#Cc1cc(C(N)=O)cc(-c2nc(N)nc3c2ncn3Cc2cc(F)cc(C(F)(F)F)c2F)c1 ; N#Cc1cc(C#N)cc(-c2nc(N)nc3c2ncn3Cc2cc(Cl)cc(C(F)(F)F)c2F)c1 ; N#Cc1cc(C(N)=O)cc(-c2nc(N)nc3c2ncn3Cc2cc(Cl)cc(C(F)(F)F)c2F)c1]

7. A pharmaceutical composition comprising the compound, stereoisomer, isotopically labeled compound, hydrate, solvate, or pharmaceutically acceptable salt thereof according to any one of claim 1 as an active ingredient.

8. A method for treating CXCR7-mediated diseases, comprising administering the compound, stereoisomer, isotopically labeled compound, hydrate, solvate, or pharmaceutically acceptable salt thereof according to claim 1 to a subject.

9. The method according to claim 8, wherein the disease is selected from fibrosis, cholestatic liver disease, inflammatory disease, angiogenic disease, cancer, autoimmune disorder, osteoporosis, pulmonary hypertension, acute ischemic injury, and thrombosis.

10. The method according to claim 9, wherein the fibrosis is fibrosis of the liver, lung, skin, kidney, heart, joint, or intestine.

11. The method according to claim 9, wherein the fibrosis is selected from the group consisting of liver fibrosis, liver cirrhosis, pulmonary fibrosis, idiopathic pulmonary fibrosis, interstitial lung disease, kidney fibrosis, myocardial fibrosis, and arthrofibrosis.

12. The method according to claim 9, wherein the cholestatic liver disease is selected from primary biliary cholangitis, primary sclerosing cholangitis, cholesterol gallstones, intrahepatic cholestasis of pregnancy, progressive familial intrahepatic cholestasis, and Alagille syndrome.

13. The method according to claim 9, wherein
the inflammatory disease is arthritis, asthma, chronic obstructive pulmonary disease, atherosclerosis, myocarditis, sarcoidosis, or sinusitis; and
the angiogenic disease is rheumatoid arthritis, psoriasis, diabetic retinopathy, retinopathy of prematurity, decreased visual acuity, corneal graft rejection, neovascular glaucoma, retrolental fibroplasia, skin flushing, Osler-Weber syndrome, myocardial neovascularization, plaque neovascularization, telangiectasia, hemophilic joint, angiofibroma, intestinal adhesions, Crohn's disease, eczema, scleroderma, diabetes, atherosclerosis, wound granulation, or keloid.

14. The method according to claim 9, wherein the cancer is selected from glioma, mesothelioma, melanoma, lymphoma, leukemia, adenocarcinoma, breast cancer, ovarian cancer, cervical cancer, leukemia, lymphoma, prostate cancer, Burkitt's lymphoma, head and neck cancer, colon cancer, colorectal cancer, non-small cell lung cancer, small cell lung cancer, esophageal cancer, gastric cancer, pancreatic cancer, hepatobiliary cancer, gallbladder cancer, small intestine cancer, rectal cancer, kidney cancer, bladder cancer, prostate cancer, genital cancer, urethral cancer, testicular cancer, cervical cancer, vaginal cancer, uterine cancer, ovarian cancer, thyroid cancer, parathyroid cancer, adrenal cancer, pancreatic endocrine cancer, glioblastoma, bone cancer, skin cancer, retinoblastoma, Hodgkin's lymphoma, and non-Hodgkin's lymphoma.

15. The method according to claim 9, wherein the autoimmune disorder is multiple sclerosis, rheumatoid arthritis, inflammatory bowel disease, systemic lupus erythematosus, lupus nephritis, interstitial cystitis, celiac disease, autoimmune encephalomyelitis, dehydration disease, osteoarthritis, or type I diabetes.

16. The method according to claim 9, wherein the pulmonary hypertension is pulmonary arterial hypertension or chronic thromboembolic pulmonary hypertension.
